(19) United States
(12) Patent Application Publication
(10) Pub. No.: US 2025/0136563 A1
(43) Pub. Date: May 1, 2025

(54) THIADIAZOLONE DERIVATIVES USEFUL AS AMPK ACTIVATOR

(71) Applicant: Betagenon AB, Umeå (SE)

(72) Inventors: Thomas EDLUND, Umeå (SE); Jacob WESTMAN, Järlåsa (SE)

(73) Assignee: Betagenon AB, Umeå (SE)

(21) Appl. No.: 18/684,154

(22) PCT Filed: Sep. 1, 2022
(86) PCT No.: PCT/GB2022/052234

§ 371 Date: Feb. 15, 2024

(30) Foreign Application Priority Data
Sep. 2, 2021 (GB) 2112529.9

(51) Int. Cl. C07D 285/08; A61K 31/433; C07D 417/12

(52) U.S. Cl. CPC C07D 285/08; A61K 31/433; C07D 417/12

(57) ABSTRACT

The invention relates to a compound of formula I, (I) wherein: R¹, R², R³ and R⁴ are as defined in the specification, or a pharmaceutically acceptable salt or solvate thereof, which compounds are useful in the treatment of a disorder or condition ameliorated by the activation of AMPK, particularly as prodrugs.

### FIELD OF THE INVENTION

The present invention relates to novel compounds, and the use of such compounds in medicine. In particular, the present invention relates to compounds that are useful in treating a disorder or condition ameliorated by the activation of AMP-activated protein kinase.

### BACKGROUND OF THE INVENTION

AMP-activated protein kinase (AMPK) is a protein kinase enzyme that consists of three protein sub-units and is activated by hormones, cytokines, exercise, and stresses that diminish cellular energy state (e.g. glucose deprivation). Activation of AMPK increases processes that generate adenosine 5′-triphosphate (ATP) (e.g., fatty-acid oxidation) and restrains others such as fatty acid-, glycerolipid- and protein-synthesis that consume ATP, but are not acutely necessary for survival. Conversely, when cells are presented with a sustained excess of glucose, AMPK activity diminishes and fatty acid-, glycerolipid- and protein-synthesis are enhanced. AMPK thus is a protein kinase enzyme that plays an important role in cellular energy homeostasis. Therefore, the activation of AMPK is coupled to glucose lowering effects and triggers several other biological effects, including the inhibition of cholesterol synthesis, lipogenesis, triglyceride synthesis, and the reduction of hyperinsulinemia.

Given the above, AMPK is a preferred target for the treatment of the metabolic syndrome and especially type 2 diabetes. AMPK is also involved in a number of pathways that are important for many different diseases (e.g. AMPK is also involved in a number of pathways that are important in CNS disorders, fibrosis, osteoporosis, heart failure and sexual dysfunction).

AMPK is also involved in a number of pathways that are important in cancer. Several tumour suppressors are part of the AMPK pathway. AMPK acts as a negative regulator of the mammalian TOR (mTOR) and EF2 pathway, which are key regulators of cell growth and proliferation. The deregulation may therefore be linked to diseases such as cancer (as well as diabetes). AMPK activators may therefore be of utility as anti-cancer drugs.

It has been shown that AMPK activator drugs (e.g. metformin and 4-chloro-N-[2-[(4-chlorophenyl)methyl]-3-oxo-1,2,4-thiadiazol-5-yl]benzamide (i.e. the compound of formula II below)) are effective at treating pain. Das and co-workers report that, following lumbar disc puncture, post injury treatment in mice with AMPK activator drugs reduces mechanical hypersensitivity (Das V, et al. Reg Anesth Pain Med 2019; 0:1-5. doi:10.1136/rapm-2019-100839). Similarly, Das and co-workers also report that early treatment with AMPK activator drugs reduces mechanical hypersensitivity in a postoperative pain model in mice (Das V, et al. Reg Anesth Pain Med 2019; 0:1-6. doi:10.1136/rapm-2019-100651). These drugs also normalize the AMPK pathway in the dorsal root ganglion. AMPK activators may therefore be used in the treatment of pain, particularly post-operative pain.

It has also been shown that hepatic steatosis may be regulated by AMPK (Zhao et al. J. Biol. Chem. 2020 295: 12279-12289). Activation of AMPK inhibits de novo lipogenesis while promoting fatty acid oxidation (β-oxidation) in the liver. AMPK activation also reduces free fatty acid release from adipose tissue and prevents hepatic steatosis. Pharmacological activation of AMPK in the liver was reported to promote beneficial effects on multiple aspects of non-alcoholic fatty liver disease (NAFLD). For example, activation of AMPK was found to improve non-alcoholic steatohepatitis (NASH) in both murine and simian animal models. Accordingly, AMPK activators may be useful in the treatment of NAFLD and NASH.

An example of an AMPK activator is 4-chloro-N-[2-[(4-chlorophenyl)methyl]-3-oxo-1,2,4-thiadiazol-5-yl]benzamide (i.e. the compound of formula II), which was first disclosed in WO 2011/004162.

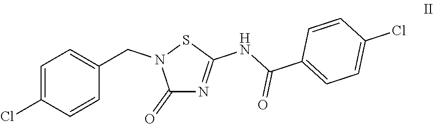 [SMILES: O=C(Nc1nc(=O)n(Cc2ccc(Cl)cc2)s1)c1ccc(Cl)cc1]

As an AMPK agonist (i.e. an AMPK activator), the compound of formula II is useful in the treatment of disorders or conditions which are ameliorated by the activation of AMPK. Such compounds may be useful in the treatment of cardiovascular disease (such as heart failure), diabetic kidney disease, type 2 diabetes, insulin resistance, non-alcoholic fatty liver disease, non-alcoholic steatohepatitis, pain, opioid addiction, obesity, cancer, inflammation (including chronic inflammatory diseases), autoimmune diseases, osteoporosis and intestinal diseases.

Although a number of AMPK activators are known, there is still a need for the development of novel compounds for the treatment of a disorder or condition ameliorated by the activation of AMPK. The inventors have now found novel compounds that are metabolised in vivo to form a known AMPK activator, with a surprising enhancement of the bioavailability of the AMPK activator. Said compounds were also found to activate AMPK.

The listing or discussion of an apparently prior-published document in this specification should not necessarily be taken as an acknowledgement that the document is part of the state of the art or is common general knowledge.

### DETAILED DESCRIPTION OF THE INVENTION

In a first aspect of the invention, there is provided a compound of formula I

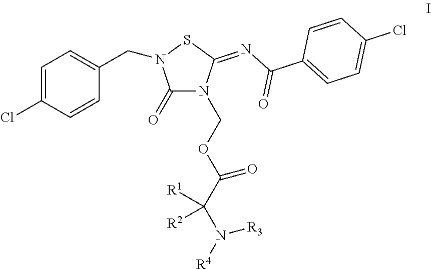

wherein:
R¹ and R² are independently selected from the group consisting of hydrogen, the sidechain of a proteinogenic amino acid, and C₁₋₆ alkyl optionally substituted by one or more substituents selected from the group consisting of —OH, —NH₂ and —C(O)NH₂;
R³ and R⁴ are independently selected from the group consisting of hydrogen, C₁₋₄alkyl optionally substituted by a phenyl group and A¹; or
R¹ (or R²) and R³ (or R⁴) together with the carbon and nitrogen atoms to which they are bound form a 4- to 6-membered heterocycloalkyl group;
A¹ represents

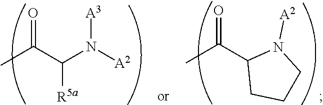

A² represents hydrogen, C₁₋₄ alkyl optionally substituted by a phenyl group,

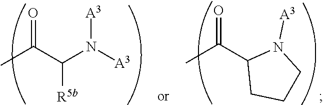

each A³ independently represents hydrogen or C₁₋₄ alkyl optionally substituted by a phenyl group; and
R^{5a} and R^{5b} are independently selected from the group consisting of hydrogen, the sidechain of a proteinogenic amino acid and C₁₋₆ alkyl optionally substituted by one or more substituents selected from the group consisting of —OH, —NH₂ and —C(O)NH₂,
or a pharmaceutically acceptable salt or solvate thereof.

These compounds, including pharmaceutically acceptable salts and solvates thereof, may be referred to herein as the “compounds of the invention”.

In preferred embodiments of the first aspect of the invention A² represents hydrogen,

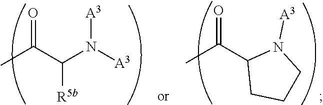

and each A³ represents hydrogen.

The compounds of the invention have been found to metabolise in vivo to form 4-chloro-N-[2-[(4-chlorophenyl)methyl]-3-oxo-1,2,4-thiadiazol-5-yl] benzamide (referred to herein as a compound of formula II) which is known to be an AMPK activator. In this regard, the compounds of the invention may be considered to be prodrugs of 4-chloro-N-[2-[(4-chlorophenyl)methyl]-3-oxo-1,2,4-thiadiazol-5-yl]benzamide.

Pharmaceutically acceptable salts of potential utility include those discussed in J. Pharmaceutical Sciences, 66: 1-19 (1977), by Berge et al. Pharmaceutically acceptable salts of the compound of formula I may be prepared in accordance with techniques that are well known to those skilled in the art.

Examples of pharmaceutically acceptable addition salts include those derived from organic acids, such as citric, tartaric, acetic (including halogenated forms of acetic acid, such as trifluoroacetic acid), malic, lactic, fumaric, benzoic, glycolic, gluconic, succinic, and arylsulphonic acids; and from mineral acids, such as hydrochloric, hydrobromic, phosphoric, metaphosphoric, nitric and sulphuric acids. In particular embodiments, the pharmaceutically acceptable salt is a citric acid salt or a trifluoroacetic acid salt of the compound of formula I.

Unless otherwise specified, alkyl groups defined herein may be straight-chain or, when there is a sufficient number (i.e. a minimum of two or three, as appropriate) of carbon atoms, be branched-chain, and/or cyclic (so forming a cycloalkyl group). When there is a sufficient number (i.e. a minimum of four) of carbon atoms, such groups may also be part cyclic (so forming a partial cycloalkyl group). For example, cycloalkyl groups that may be mentioned include cyclopropyl, cyclobutyl, cyclopentyl and cyclohexyl. Similarly, part cyclic alkyl groups (which may also be referred to as “part cycloalkyl” groups) that may be mentioned include cyclopropylmethyl.

Heterocycloalkyl groups that may be mentioned include non-aromatic monocyclic heterocycloalkyl groups in which at least one (e.g. one to four) of the atoms in the ring system is other than carbon (i.e. a heteroatom, e.g. sulfur, oxygen or, particularly, nitrogen), and in which the total number of atoms in the ring system is from four to six. Carbon atoms of the heterocycloalkyl groups mentioned herein may be substituted by one or more ═O substituents.

For the avoidance of doubt, in cases in which the identity of two or more substituents in a compound of formula I may be the same, the actual identities of the respective substituents are not in any way interdependent.

Where groups are referred to herein as being optionally substituted it is specifically contemplated that such optional substituents may be not present (i.e. references to such optional substituents may be removed), in which case the optionally substituted group may be referred to as being unsubstituted in certain embodiments.

Pharmaceutically acceptable salts of the compound of formula I may be prepared in accordance with techniques that are well known to those skilled in the art. For example, the compound of formula I may be reacted with the appropriate organic acid or mineral acid. Salt switching techniques may also be used to convert one salt into another salt.

The compounds disclosed herein may exist in unsolvated as well as solvated forms with pharmaceutically acceptable solvents such as water and ethanol, and it is intended that the invention embraces both solvated and unsolvated forms of the compounds of the invention.

The term “solvate” refers to a complex of variable stoichiometry formed by a solute and solvent. Such solvents for the purpose of the invention may not interfere with the biological activity of the solute. Examples of suitable solvents include, but are not limited to, water, methanol, ethanol, and acetic acid. Solvates in which water is the solvent molecule are typically referred to as hydrates. Hydrates include compositions containing stoichiometric amounts of water, as well as compositions containing variable amounts of water.

Compounds of formula I contain double bonds and may thus exist as E (entgegen) and Z (zusammen) geometric isomers about each individual double bond. All such isomers and mixtures thereof are included within the scope of the invention.

Compounds of formula I may exist as regioisomers and may also exhibit tautomerism. All tautomeric forms and mixtures thereof are included within the scope of the invention.

The present invention also embraces isotopically-labelled compounds of formula I which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the invention. Hence, the compounds of formula I also include deuterated compounds, i.e. compounds of formula I in which one or more hydrogen atoms are replaced by the hydrogen isotope deuterium.

The skilled person will appreciate that compounds of the invention that are the subject of this invention include those that are stable. That is, compounds of the invention include those that are sufficiently robust to survive isolation from, e.g., a reaction mixture to a useful degree of purity.

Throughout this specification, structures may or may not be presented with chemical names. Where any question arises as to nomenclature, the structure prevails. Where it is possible for the compound to exist as a tautomer (e.g. In an alternative resonance form) the depicted structure represents one of the possible tautomeric forms, wherein the actual tautomeric form(s) observed may vary depending on environmental factors such as solvent, temperature or pH. All tautomeric (and resonance) forms and mixtures thereof are included within the scope of the invention.

Unless indicated otherwise, all technical and scientific terms used herein will have their common meaning as understood by one of ordinary skill in the art to which this invention pertains.

For the avoidance of doubt, the skilled person will understand that references herein to particular aspects of the invention (such as the first aspect of the invention) will include references to all embodiments and particular features thereof, which embodiments and particular features may be taken in combination to form further embodiments and features of the invention.

Amino acids are organic compounds that contain amino (—NH₂) and carboxyl (—CO₂H) functional groups, along with (in most cases) a sidechain. Alpha amino acids are those where the amino and carboxyl functional groups are bonded to the same carbon atom (i.e. the α-carbon atom). Particular alpha amino acids that might be mentioned are the proteinogenic amino acids. Proteinogenic amino acids are amino acids that are incorporated biosynthetically into proteins during translation. The proteinogenic amino acids are glycine (Gly), alanine (Ala), arginine (Arg), asparagine (Asn), aspartic acid (Asp), cysteine (Cys), glutamic acid (Glu), glutamine (Gin), histidine (His), isoleucine (Ile), leucine (Leu), lysine (Lys), methionine (Met), phenylalanine (Phe), proline (Pro), pyrrolysine (Pyl), selenocysteine (Sec), serine (Ser), threonine (Thr), tryptophan (Trp), tyrosine (Tyr), and valine (Val).

Thus, the term “sidechain of a proteinogenic amino acid” refers to the group other than hydrogen that is bonded to the α-carbon atom of a proteinogenic amino acid. In the context of the present invention the invention, sidechains of one or more proteinogenic amino acids may be located at any of the R¹, R², R^{5a} or R^{5b} positions of the compound of formula I. For the avoidance of doubt, the compounds of the invention may contain multiple different sidechains in these positions, i.e. the prodrug of compound II may contain a chain having two or three different amino acids linked to the thiadiazole ring. Particular examples of proteinogenic amino acid sidechains include methyl substituted with an imidazolyl or indolyl group (for histidine and tryptophan), propyl substituted with a guanidino group (for arginine) and butyl substituted with an amino group (for lysine).

Particular compounds of the invention that may be mentioned are those where R¹ and R² are the same. For example, R¹ and R² may both be hydrogen.

Other compounds of the invention that may be mentioned are those where R¹ and R² are the different. For example, R¹ may be the side chain of a proteinogenic amino acid and R² may be hydrogen.

Thus, compounds of the first aspect of the invention may contain one or more asymmetric carbon atoms and may therefore exhibit optical and/or diastereoisomerism. In particular, compounds of formula I where R¹ and R² are different (e.g. where R¹ is methyl and R² is hydrogen) may be such that the carbon substituted with R¹ and R² is in the L-configuration or the D-configuration, as understood by those skilled in the art. Where compounds contain an R⁴ and/or an R⁵ group, the chiral centres to which those R^{5a} and R^{5b} groups are bound may also, independently, be in either the L-configuration or the D-configuration.

In particular embodiments, when R¹ and R² are different the carbon substituted with R¹ and R² is in the L-configuration. Alternatively, when R¹ and R² are different the carbon substituted with R¹ and R² may be in the D-configuration. For example, the compound of formula I may be a compound of formula I-A or a compound of formula I-B

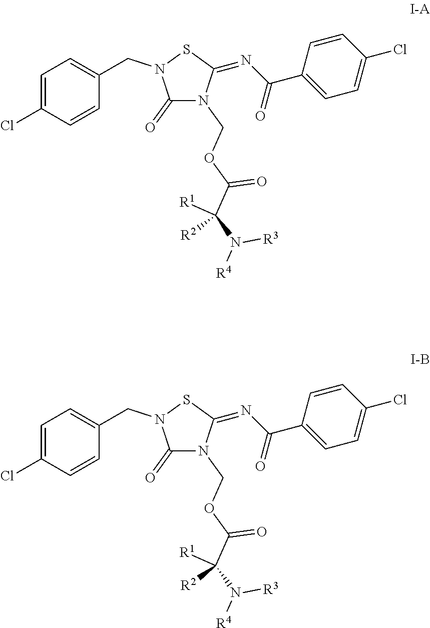

wherein R¹, R², R³ and R⁴ are as described herein (i.e. as described in the first aspect of the invention, including all embodiments and particular features, and combinations thereof). Particular examples of compounds of the invention are those in which R¹ and R² are both hydrogen or the carbon substituted with R¹ and R² is in the L-configuration, e.g. when one of R¹ and R² is hydrogen and the other is a sidechain of a proteinogenic amino acid.

Similarly, when R³ or R⁴ represents A¹, A¹ may represent

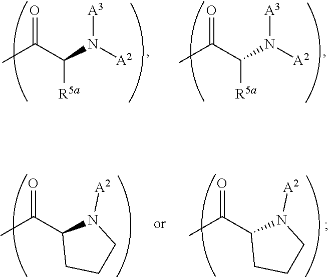

and
A² may represent hydrogen, C₁₋₄ alkyl optionally substituted by a phenyl group,

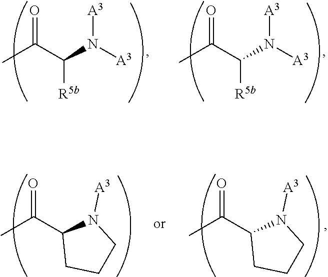

wherein A³, R^{5a} and R^{5b} are as described herein (i.e. as described in the first aspect of the invention, including all embodiments and particular features, and combinations thereof). Particular examples of compounds of the invention are those in which A¹ represents a proteinogenic amino acid or two linked proteinogenic amino acids.

Preferred examples of compounds of the invention that may be mentioned include those where:

A² represents hydrogen,

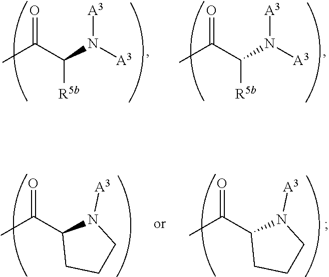

each A³ represents hydrogen; and R^{5b} is as described herein.

Preferably, R¹ is selected from the group consisting of hydrogen, the sidechain of a proteinogenic amino acid, and C₁₋₆ alkyl optionally substituted by one or more substituents selected from the group consisting of —OH, —NH₂ and —C(O)NH₂, or R¹ and R³ are linked to form a pyrrolidine ring (as is found in proline); and R² is hydrogen.

In particular embodiments, R² is hydrogen, and R¹ is hydrogen or the sidechain of a proteinogenic amino acid selected from the group consisting of Glu and, particularly, Arg, His, Lys, Ser, Thr, Asn, Gln, Cys, Sec, Ala, Ile, Leu, Met, Phe, Trp, Tyr, Asp and Val; or

R² is hydrogen, and R¹ and R³ together with the carbon and nitrogen atoms to which they are bound form a pyrrolidine ring.

In particular embodiments, R² is hydrogen, and R¹ is hydrogen or the sidechain of an amino acid selected from the group consisting of Arg, His, Lys and Trp (e.g. Arg, His and Lys); or

R² is hydrogen, and R¹ and R³ together with the carbon and nitrogen atoms to which they are bound form a pyrrolidine ring.

In a further embodiment, R² is hydrogen, and R¹ is hydrogen or the sidechain of an amino acid selected from the group consisting of Lys, Ala, Ile, Phe, Leu, Ser and Asp; or

R² is hydrogen, and R¹ and R³ together with the carbon and nitrogen atoms to which they are bound form a pyrrolidine ring.

Preferably, R² is hydrogen.

Particular embodiments of the invention include compounds of formula I wherein:
R¹ is hydrogen or the sidechain of an amino acid selected from the group consisting of Arg, His, Lys and Trp (e.g. Arg, His and Lys); or
R¹ and R³ together with the carbon and nitrogen atoms to which they are bound form a pyrrolidine ring; and
R² is hydrogen.

In other embodiments, R³ and R⁴ are independently selected from the group consisting of hydrogen, C₁₋₃ alkyl optionally substituted by one or more phenyl groups (e.g. a methyl or benzyl group),

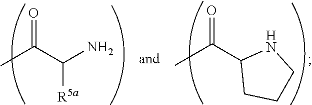

and R^{5a} is hydrogen or the sidechain of a proteinogenic amino acid. In preferred embodiments, R³ and R⁴ are independently selected from the group consisting of hydrogen and C₁₋₃ alkyl optionally substituted by one or more phenyl groups (e.g. R³ and R⁴ are independently hydrogen, methyl or benzyl). Preferably, R³ and R⁴ are independently selected from the group consisting of hydrogen and methyl (e.g. R³ and R⁴ are both methyl).

In other preferred embodiments, R³ and R⁴ are independently selected from the group consisting of hydrogen, C₁₋₃ alkyl optionally substituted by one or more phenyl groups (e.g. a methyl or benzyl group),

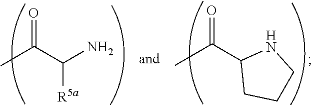

or
R¹ and R³ together with the carbon and nitrogen atoms to which they are bound form a pyrrolidine ring and R⁴ is selected from the group consisting of hydrogen, C₁₋₃ alkyl optionally substituted by one or more phenyl groups,

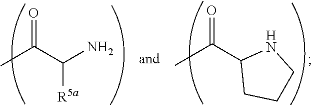

and R^{5a} is the sidechain of a proteinogenic amino acid (e.g. Phe, Val, Lys, Asp or Arg).

Preferred compounds of the invention that may be mentioned include those in which:
R¹ is hydrogen or the sidechain of a proteinogenic amino acid selected from the group consisting of Glu and, particularly, Arg, His, Lys, Ser, Thr, Asn, Gln, Cys, Sec, Ala, Ile, Leu, Met, Phe, Trp, Tyr, Asp and Val (e.g. Lys, Ala, Ile, Phe, Leu, Ser and Asp; or Arg, His, Lys and Trp);
R² represents hydrogen;
R³ and R⁴ are independently selected from the group consisting of hydrogen and C₁₋₃ alkyl optionally substituted by one or more phenyl groups (e.g. methyl); or
R¹ and R³ together with the carbon and nitrogen atoms to which they are bound form a pyrrolidine ring.

Other preferred compounds of the invention that may be mentioned include those in which: R² represents hydrogen;
R¹ is hydrogen or the sidechain of a proteinogenic amino acid selected from the group consisting of Glu, Arg, His, Lys, Ser, Thr, Asn, Gln, Cys, Sec, Ala, Ile, Leu, Met, Phe, Trp, Tyr, Asp and Val (e.g. Lys, Ala, Ile, Phe, Leu, Ser and Asp);
R³ and R⁴ are independently selected from the group consisting of hydrogen, C₁₋₃ alkyl optionally substituted by one or more phenyl groups (e.g. a methyl or benzyl group),

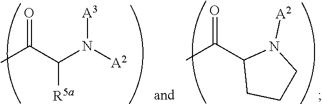

or
R¹ and R³ together with the carbon and nitrogen atoms to which they are bound form a pyrrolidine ring, and R⁴ is selected from the group consisting of hydrogen, C₁₋₃ alkyl optionally substituted by one or more phenyl groups (e.g. a methyl or benzyl group),

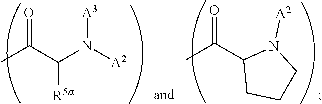

R^{5a} is hydrogen or the sidechain of a proteinogenic amino acid selected from the group consisting of Glu, Arg, His, Lys, Ser, Thr, Asn, Gln, Cys, Sec, Ala, Ile, Leu, Met, Phe, Trp, Tyr, Asp and Val (e.g. Phe, Val, Lys, Asp and Arg); and
A² and A³ are independently as defined herein.

In embodiments of the invention where A² represents

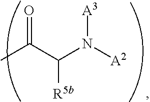

R^{5b} is preferably hydrogen or the sidechain of a proteinogenic amino acid selected from the group consisting of Glu, Arg, His, Lys, Ser, Thr, Asn, Gln, Cys, Sec, Ala, Ile, Leu, Met, Phe, Trp, Tyr, Asp and Val (e.g. Phe, Val, Lys, Asp and Arg); and each A³ is independently selected from the group consisting of hydrogen and C₁₋₃ alkyl (e.g. methyl).

As is stated above, the compounds of the invention have been found to metabolise in vivo to form 4-chloro-N-[2-[(4-chlorophenyl)methyl]-3-oxo-1,2,4-thiadiazol-5-yl] benzamide (referred to herein as a compound of formula II). The precise mechanisms by which the compounds of the invention are converted to the compound of formula II may nevertheless differ for different compounds of the invention. Without wishing to be bound by theory, it is believe that the compounds of the invention decompose into the compound of formula II in two steps. First the ester is hydrolysed (chemically or enzymically) to form the N-hydroxymethyl intermediate (referred to elsewhere herein as the compound of formula III), and then that intermediate is converted into the compound of formula II.

The applicant has found that some compounds are hydrolysed more rapidly under acidic conditions (e.g. conditions that would be expected to exist in the human stomach) whereas other compounds are hydrolysed more rapidly under more alkaline conditions (e.g. conditions that are akin that in the small intestine).

Compounds of the invention that are hydrolysed more rapidly under alkaline conditions (e.g. conditions that are akin to that in the small intestine) than under acidic conditions may be advantageous in that they may offer additional clinical benefits, for example they may be useful for patients that are unable to achieve adequate conversion in the stomach (e.g. unable to achieve conversion at the required rate or to the required extent). Such patients may suffer from inadequate function of esterase enzymes, for example. In addition, the intermediate that is formed as a result of the hydrolysis (i.e. Compound of formula III) may be more readily taken up in the gut compared to Compound of formula II, thus the formation of Compound of formula III in the intestine may be advantageous for achieving optimal systemic exposure for the patient.

We have found that compounds of the invention which contain a lipophilic (alkyl) group at R¹/R² have a relatively high stability at pH 7.4. The stability is lower at this pH when R¹/R² groups are H or a —NH₂ containing amino acid side chain (e.g. glycine-based or lysine-based). The stability is very low at this pH when one of the R¹/R² groups is an electron withdrawing amino acid side chain (e.g. aspartic acid-based, arginine-based or glutamic acid-based side chain).

Particular compounds that may be mentioned in this respect are compounds of formula A:

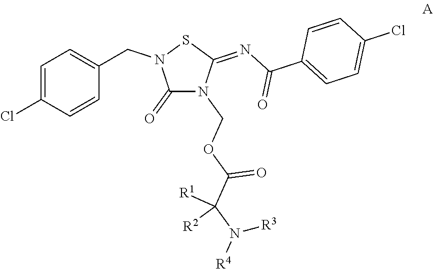

wherein:
(i) R¹ represents hydrogen, R² represents the sidechain of aspartic acid, the sidechain of arginine or the sidechain of glutamic acid, R³ is hydrogen or methyl, and R⁴ hydrogen or methyl; or
(ii) R¹, R² and R³ represent hydrogen, and R⁴ represents A¹,
   A¹ represents

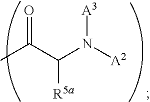

wherein A² is hydrogen or methyl, A³ is hydrogen or methyl, and R^{5a} represents the sidechain of aspartic acid, the sidechain of arginine or the sidechain of glutamic acid;
   or a pharmaceutically acceptable salt or solvate thereof.

Further compounds that may be mentioned in this respect are compounds of formula B:

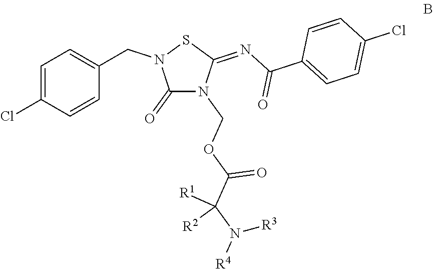

wherein:
(i) R¹ represents hydrogen, R² represents the sidechain of aspartic acid or the side chain of arginine, R³ is hydrogen or methyl, and R⁴ hydrogen or methyl; or
(ii) R¹, R² and R³ represent hydrogen, R⁴ represents A¹;
   A¹ represents

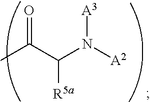

A² is hydrogen or methyl, A³ is hydrogen or methyl, and R⁵ represents the sidechain of aspartic acid or the side chain of arginine;
   or a pharmaceutically acceptable salt or solvate thereof.

Still further particular compounds that may be mentioned in this respect are compounds of formula C,

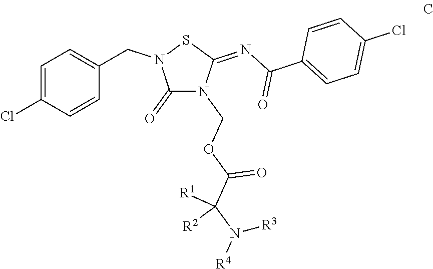

wherein
(i) R¹, R³ and R⁴ represent hydrogen, and R² represents the sidechain of aspartic acid, the sidechain of arginine or the sidechain of glutamic acid; or
(ii) R¹, R² and R³ represent hydrogen, and R⁴ represents A¹,
   A¹ represents

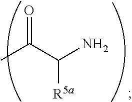

and
   R^{5a} represents the sidechain of aspartic acid, the sidechain of arginine or the sidechain of glutamic acid;
   or a pharmaceutically acceptable salt or solvate thereof.

In another embodiment, the compound of the invention is a compound of formula I in which R² is hydrogen, and R¹ is the sidechain of aspartic acid, optionally wherein R³ and R⁴ are independently hydrogen or methyl.

Yet further compounds that may be mentioned in this respect are:

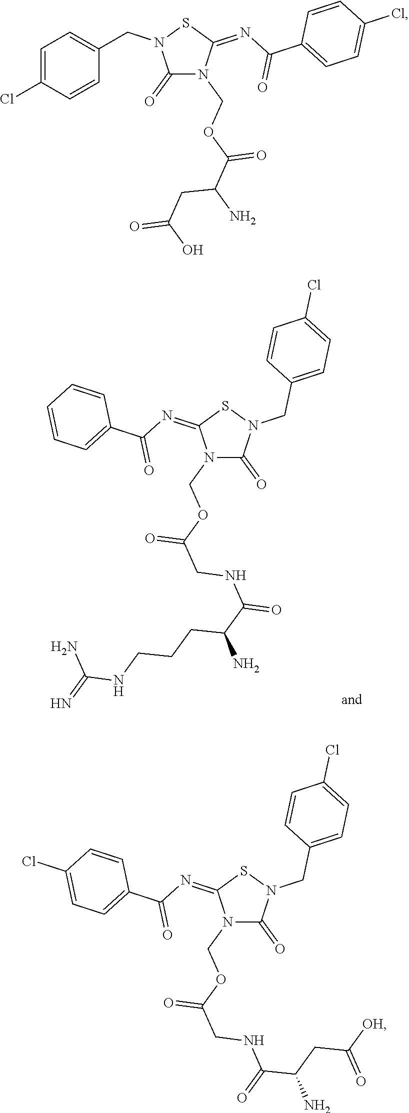 [SMILES: NC(CC(=O)O)C(=O)OCn1c(=O)n(Cc2ccc(Cl)cc2)s/c1=N/C(=O)c1ccc(Cl)cc1 ; N=C(N)NCCC[C@H](N)C(=O)NCC(=O)OCn1c(=O)n(Cc2ccc(Cl)cc2)s/c1=N/C(=O)c1ccccc1 ; N[C@@H](CC(=O)O)C(=O)NCC(=O)OCn1c(=O)n(Cc2ccc(Cl)cc2)s/c1=N/C(=O)c1ccc(Cl)cc1]

and pharmaceutically acceptable salts or solvates thereof.

In further embodiments, the invention also relates to compounds of formula I as defined herein (including compounds of formula I as defined according to any of the preferred embodiments set out herein) provided that:
(i) the compound is not a compound of formula A; or
(ii) the compound is not a compound of formula B; or
(iii) the compound is not a compound of formula C; or
(iv) the compound is not selected from the group consisting of:

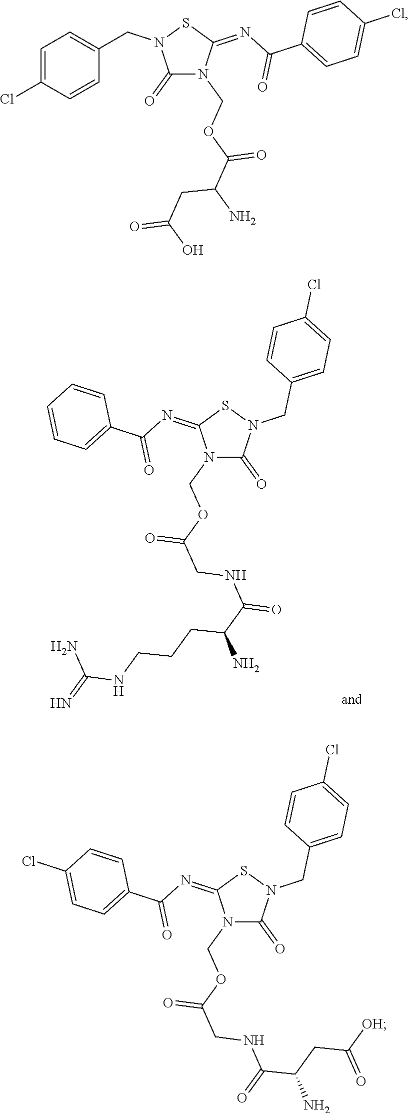 [SMILES: NC(CC(=O)O)C(=O)OCn1c(=O)n(Cc2ccc(Cl)cc2)s/c1=N/C(=O)c1ccc(Cl)cc1 ; N=C(N)NCCC[C@H](N)C(=O)NCC(=O)OCn1c(=O)n(Cc2ccc(Cl)cc2)s/c1=N/C(=O)c1ccccc1 ; N[C@@H](CC(=O)O)C(=O)NCC(=O)OCn1c(=O)n(Cc2ccc(Cl)cc2)s/c1=N/C(=O)c1ccc(Cl)cc1]

or
(v) the compound is not a compound of formula I in which R² is hydrogen, and R¹ is the sidechain of aspartic acid (optionally wherein R³ and R⁴ are independently hydrogen or methyl); or
(vi) the compound is not:

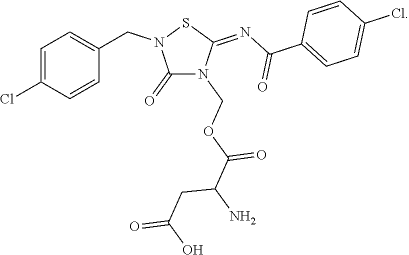 [SMILES: NC(CC(=O)O)C(=O)OCn1c(=O)n(Cc2ccc(Cl)cc2)s/c1=N/C(=O)c1ccc(Cl)cc1]

Particularly preferred compounds of the invention are:

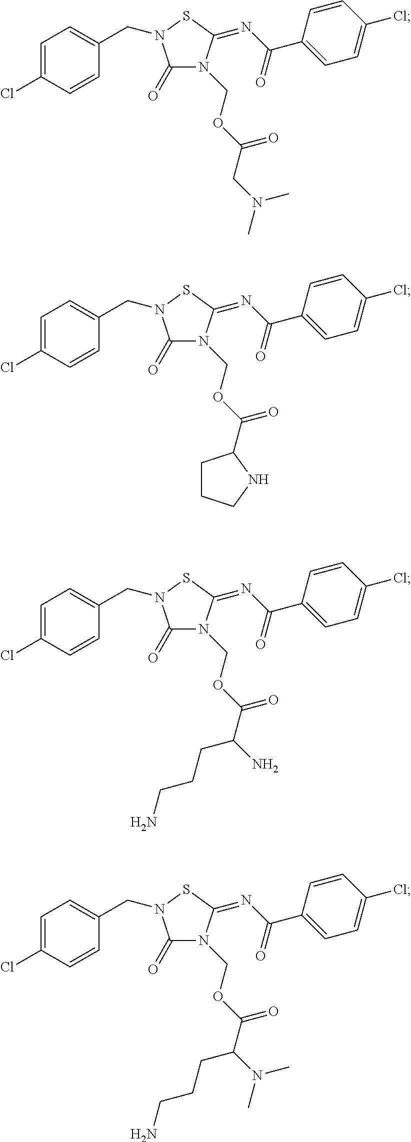 [SMILES: CN(C)CC(=O)OCn1c(=O)n(Cc2ccc(Cl)cc2)s/c1=N/C(=O)c1ccc(Cl)cc1 ; O=C(/N=c1/sn(Cc2ccc(Cl)cc2)c(=O)n1COC(=O)C1CCCN1)c1ccc(Cl)cc1 ; NCCCC(N)C(=O)OCn1c(=O)n(Cc2ccc(Cl)cc2)s/c1=N/C(=O)c1ccc(Cl)cc1 ; CN(C)C(CCCN)C(=O)OCn1c(=O)n(Cc2ccc(Cl)cc2)s/c1=N/C(=O)c1ccc(Cl)cc1]

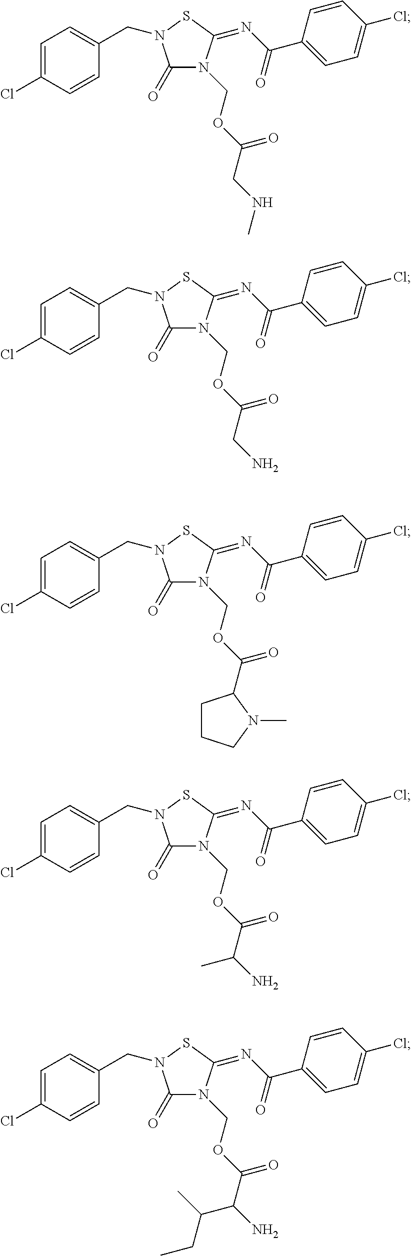 [SMILES: CNCC(=O)OCn1c(=O)n(Cc2ccc(Cl)cc2)s/c1=N/C(=O)c1ccc(Cl)cc1 ; NCC(=O)OCn1c(=O)n(Cc2ccc(Cl)cc2)s/c1=N/C(=O)c1ccc(Cl)cc1 ; CN1CCCC1C(=O)OCn1c(=O)n(Cc2ccc(Cl)cc2)s/c1=N/C(=O)c1ccc(Cl)cc1 ; CC(N)C(=O)OCn1c(=O)n(Cc2ccc(Cl)cc2)s/c1=N/C(=O)c1ccc(Cl)cc1 ; CCC(C)C(N)C(=O)OCn1c(=O)n(Cc2ccc(Cl)cc2)s/c1=N/C(=O)c1ccc(Cl)cc1]

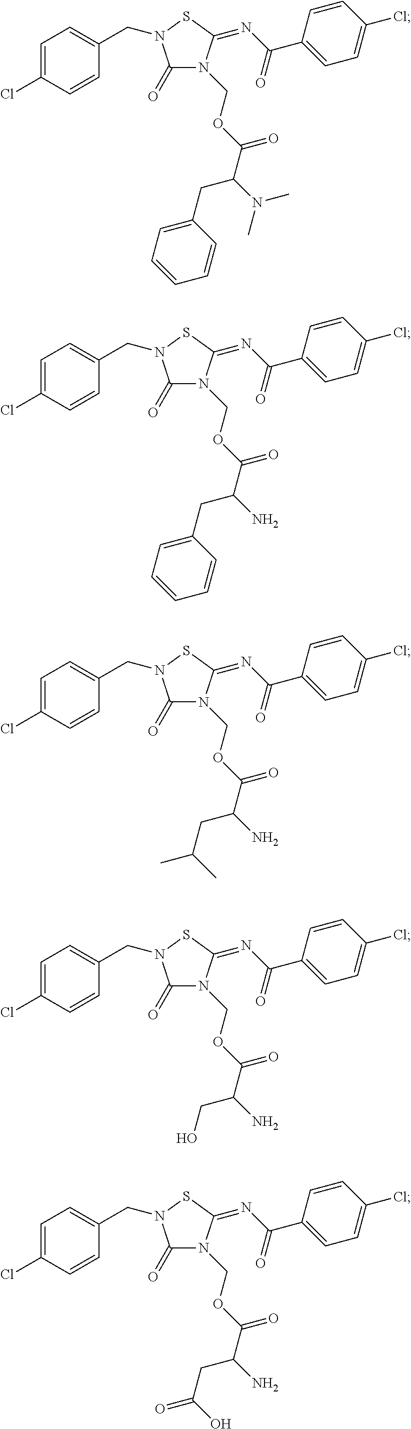 [SMILES: CN(C)C(Cc1ccccc1)C(=O)OCn1c(=O)n(Cc2ccc(Cl)cc2)s/c1=N/C(=O)c1ccc(Cl)cc1 ; NC(Cc1ccccc1)C(=O)OCn1c(=O)n(Cc2ccc(Cl)cc2)s/c1=N/C(=O)c1ccc(Cl)cc1 ; CC(C)CC(N)C(=O)OCn1c(=O)n(Cc2ccc(Cl)cc2)s/c1=N/C(=O)c1ccc(Cl)cc1 ; NC(CO)C(=O)OCn1c(=O)n(Cc2ccc(Cl)cc2)s/c1=N/C(=O)c1ccc(Cl)cc1 ; NC(CC(=O)O)C(=O)OCn1c(=O)n(Cc2ccc(Cl)cc2)s/c1=N/C(=O)c1ccc(Cl)cc1]

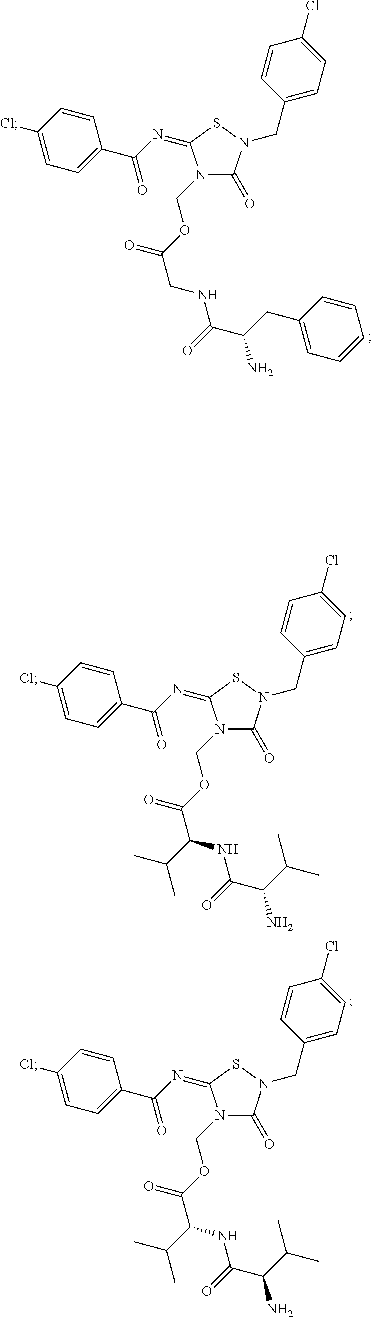 [SMILES: N[C@@H](Cc1ccccc1)C(=O)NCC(=O)OCn1c(=O)n(Cc2ccc(Cl)cc2)s/c1=N/C(=O)c1ccc(Cl)cc1 ; ";" ; CC(C)[C@H](N)C(=O)N[C@H](C(=O)OCn1c(=O)n(Cc2ccc(Cl)cc2)s/c1=N/C(=O)c1ccc(Cl)cc1)C(C)C ; ";" ; CC(C)[C@@H](N)C(=O)N[C@@H](C(=O)OCn1c(=O)n(Cc2ccc(Cl)cc2)s/c1=N/C(=O)c1ccc(Cl)cc1)C(C)C ; ";"]

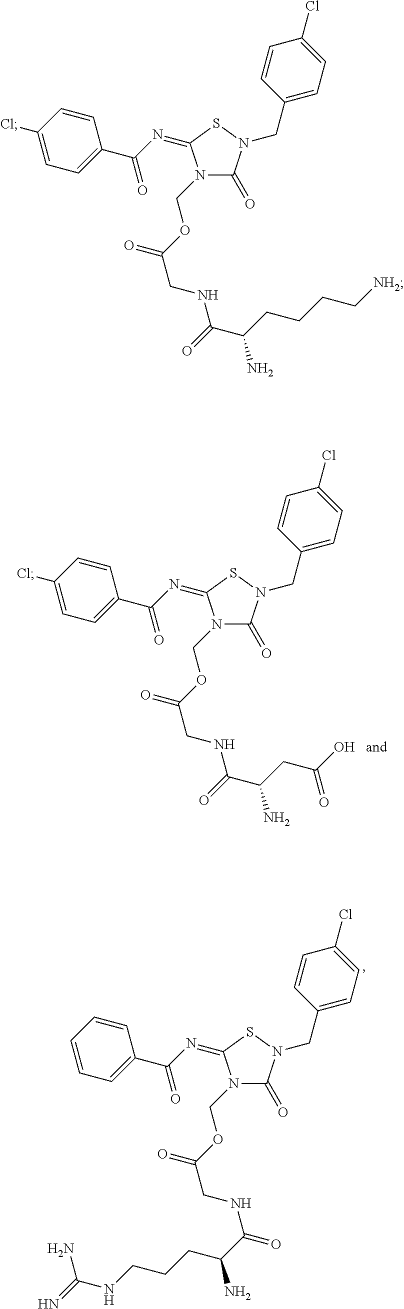 [SMILES: NCCCC[C@H](N)C(=O)NCC(=O)OCn1c(=O)n(Cc2ccc(Cl)cc2)s/c1=N/C(=O)c1ccc(Cl)cc1 ; N[C@@H](CC(=O)O)C(=O)NCC(=O)OCn1c(=O)n(Cc2ccc(Cl)cc2)s/c1=N/C(=O)c1ccc(Cl)cc1 ; "and" ; N=C(N)NCCC[C@H](N)C(=O)NCC(=O)OCn1c(=O)n(Cc2ccc(Cl)cc2)s/c1=N/C(=O)c1ccccc1 ; ","]

or a pharmaceutically acceptable salt or solvate thereof.

The compounds of the invention may be considered to be prodrugs of 4-chloro-N-[2-[(4-chlorophenyl)methyl]-3-oxo-1,2,4-thiadiazol-5-yl]benzamide as they have been found to break down in vivo to form said compound. 4-Chloro-N-[2-[(4-chlorophenyl)methyl]-3-oxo-1,2,4-thiadiazol-5-yl]benzamide may be depicted as the compound of formula II

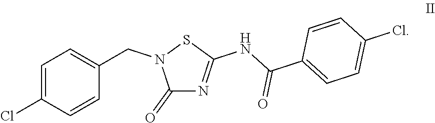 [SMILES: O=C(Nc1nc(=O)n(Cc2ccc(Cl)cc2)s1)c1ccc(Cl)cc1]

The term “prodrug” refers to a compound that, following oral or parenteral administration, is metabolised in vivo to yield a pharmalogically active compound in an experimentally-detectable amount, and within a predetermined time (e.g. within a dosing interval of between 6 and 24 hours (i.e. once to four times daily)). For the avoidance of doubt, the term “parenteral” administration includes all forms of administration other than oral administration. General information on prodrugs may be found e.g. In Bundegaard, H. “Design of Prodrugs” p. I-92, Elsevier, New York-Oxford (1985).

It has been found that administration of the compounds of the invention surprisingly resulted in a significant increase in the bioavailability and systemic exposure of 4-chloro-N-[2-[(4-chlorophenyl)methyl]-3-oxo-1,2,4-thiadiazol-5-yl]benzamide as compared to the bioavailability and systemic exposure observed following administration of 4-chloro-N-[2-[(4-chlorophenyl)methyl]-3-oxo-1,2,4-thiadiazol-5-yl]benzamide.

The bioavailability of a drug is the amount of an administered dose that reaches the systemic circulation in that drug form. Sufficient bioavailability is important to achieve a therapeutically active concentration at the site of action. An improvement (i.e. an increase) in bioavailability may be demonstrated by measuring the Cₘₐₓ or the area under the curve (AUC) in the blood of a subject following administration of the compound (or pharmaceutical formulation thereof) to that subject. The compounds and formulations of the invention are useful in the therapies described herein in a subject in need of such therapy. Subjects that may be mentioned include animals, such as mammals. Particular mammals that might be mentioned include, for example, primates (e.g., humans, male or female), cows, horses, dogs and cats. Preferably the subject is a human.

The terms “Cₘₐₓ” and “AUC” will be well understood by the person skilled in the art to refer, in the present context, to the peak plasma concentration of 4-chloro-N-[2-[(4-chlorophenyl)methyl]-3-oxo-1,2,4-thiadiazol-5-yl]benzamide after administration (e.g. to a human subject) and the integral of the concentration/time curve for that substance following the administration of the compound of the invention (or formulation thereof), respectively.

It has been found that administration of a compound of the invention resulted in a particularly enhanced bioavailability of 4-chloro-N-[2-[(4-chlorophenyl)methyl]-3-oxo-1,2,4-thiadiazol-5-yl]benzamide in vivo, as is evidenced by the data in the examples. These data show that plasma exposure of 4-chloro-N-[2-[(4-chlorophenyl)methyl]-3-oxo-1,2,4-thiadiazol-5-yl]benzamide is increased when a compound of the invention is administered to a mammalian subject as compared to the plasma exposure observed following administration of 4-chloro-N-[2-[(4-chlorophenyl)methyl]-3-oxo-1,2,4-thiadiazol-5-yl]benzamide.

Thus, administration of the compounds of the invention is capable of increasing the bioavailability of 4-chloro-N-[2-[(4-chlorophenyl)methyl]-3-oxo-1,2,4-thiadiazol-5-yl]benzamide compared to administration of 4-chloro-N-[2-[(4-chlorophenyl)methyl]-3-oxo-1,2,4-thiadiazol-5-yl]benzamide. In this context, the phrase “increasing the bioavailability” means that administration of the compound of the invention results in a larger systemically available fraction of 4-chloro-N-[2-[(4-chlorophenyl)methyl]-3-oxo-1,2,4-thiadiazol-5-yl]benzamide in vivo compared to administration of 4-chloro-N-[2-[(4-chlorophenyl)methyl]-3-oxo-1,2,4-thiadiazol-5-yl]benzamide. The increase in the systemically available fraction of 4-chloro-N-[2-[(4-chlorophenyl)methyl]-3-oxo-1,2,4-thiadiazol-5-yl]benzamide may be at least about 10%, (at least) about 20%, about 30%, about 40%, about 50%, about 60%, about 70%, about 80%, about 90%, about 100% (i.e. 2-fold), about 200% (i.e. 3-fold), about 300% (i.e. 4-fold), about 400% (i.e. 5-fold), about 500% (i.e. 6-fold), about 600% (i.e. 7-fold), about 700% (i.e. 8-fold), about 800% (i.e. 9-fold) or about 900% (i.e. 10-fold).

The improvement of the bioavailability provided by the compounds of the invention may be demonstrated using suitable methods known in the art. For example, the improvement in bioavailability may be demonstrated by comparing the pharmacokinetic data (e.g. AUC data) for a subject who has been administered a compound of the invention with the corresponding data for a subject who has been administered 4-chloro-N-[2-[(4-chlorophenyl)methyl]-3-oxo-1,2,4-thiadiazol-5-yl]benzamide.

As indicated by the examples, compounds of the invention have enhanced stability under acidic conditions (e.g. at pH 1.2). Thus, the compounds of the invention are suited to being prepared into pharmaceutical formulations for oral administration to patients where passage through the stomach is required.

The compounds of the invention have also been found to be surprisingly effective at activating AMPK, as is evidenced by the data in the examples. As AMPK activators (i.e. AMPK agonists), the compounds of the invention may be useful in the treatment of disorders or conditions which are ameliorated by the activation of AMPK. Such compounds may therefore be useful in the treatment of the particular diseases described herein.

The compounds of the invention may be prepared from 4-chloro-N-[2-[(4-chlorophenyl)methyl]-3-oxo-1,2,4-thiadiazol-5-yl]benzamide. 4-Chloro-N-[2-[(4-chlorophenyl)methyl]-3-oxo-1,2,4-thiadiazol-5-yl]benzamide may, in turn, be prepared in accordance with techniques that are well known to those skilled in the art. For example, 4-chloro-N-[2-[(4-chlorophenyl)methyl]-3-oxo-1,2,4-thiadiazol-5-yl]benzamide may be made in accordance with the techniques described in international patent application WO 2011/004162, and all of its content is hereby incorporated by reference.

Compounds of the invention as described herein may be prepared in accordance with techniques that are well known to those skilled in the art, such as those described in the examples provided hereinafter.

Compounds of formula I may be obtained by analogy with the processes known in the literature, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991.

For example, there is provided a process for the preparation of a compound of the invention as hereinbefore defined, which process comprises:
(i) reaction of a compound of formula III,

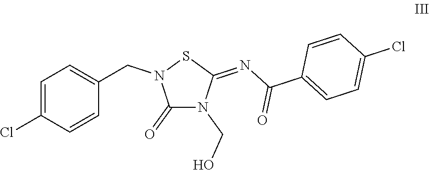 [SMILES: O=C(/N=c1/sn(Cc2ccc(Cl)cc2)c(=O)n1CO)c1ccc(Cl)cc1]

with a compound of formula IV,

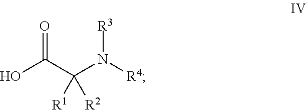

or
(ii) reaction of a compound of formula V,

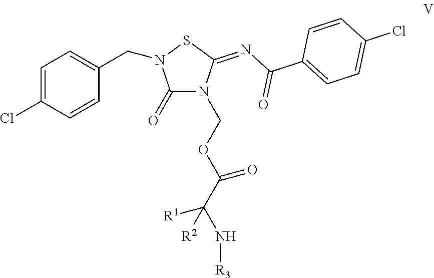

with a compound of formula IV,

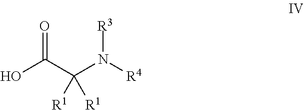

wherein R¹ and R² are as defined hereinabove and R³ and R⁴ are as defined hereinabove or independently represent a suitable protecting group (e.g. Boc), in the presence of a suitable base (e.g. 4-dimethylaminopyridine), a suitable amide coupling agent (e.g. N,N′-dicyclohexylcarbodiimide) and a suitable solvent (e.g. tetrahydrofuran) according to procedures know to the person skilled in the art.

For the avoidance of doubt, the processes described herein for preparing compounds of the invention embrace those processes which comprise reaction of a compound of formula III with up to three (e.g. two) compounds of formula IV in a sequential manner.

For example, we include those processes which comprise:
(i) reacting a compound of formula III with a compound of formula IV, according to the processes described herein;
(ii) optionally performing one or more deprotection steps;
(iii) reacting the product of step (i) or step (ii) with a compound of formula IV, according to the processes described herein;
(iv) optionally performing one or more deprotection steps;
(v) optionally reacting the product of step (iii) or step (iv) with a compound of formula IV, according to the processes described herein; and
(vi) optionally performing one or more deprotection steps.

In the above embodiment, the compound of formula IV may be the same or different in each of steps (i), (iii) and (v).

It will be appreciated by those skilled in the art that, in the processes described above and hereinafter, the functional groups of intermediate compounds may need to be protected by protecting groups. For example, in particular embodiments, in a compound of formula IV one of R³ and R⁴ may represent hydrogen and the other may represent a suitable protecting group (e.g. Boc). The protection and deprotection of functional groups may take place before or after the above-mentioned reactions.

Protecting groups may be removed in accordance with techniques that are well known to those skilled in the art and as described hereinafter. For example, protected compounds/intermediates described herein may be converted chemically to unprotected compounds using standard deprotection techniques. The use of protecting groups is fully described in “Protective Groups in Organic Synthesis”, 3rd edition, T. W. Greene & P. G. M. Wutz, Wiley-Interscience (1999).

Specific transformation steps that may be employed in order to form compounds of formula I therefore include deprotection steps, such as deprotection of an N-Boc protecting group by reaction in the presence of an acid, or, a hydroxy group protected as a silyl ether (e.g. a tert-butyl-dimethylsilyl protecting group) may be deprotected by reaction with an acid or a source of fluoride ions, e.g. by employing the reagent tetrabutylammonium fluoride (TBAF).

The compound of formula III may be obtained by conventional synthetic procedures, in accordance with standard techniques, from available starting materials using appropriate reagents and reaction conditions. For example, the compound of formula III (referred to in the examples as Compound 2) may be prepared by reaction of a compound of formula II with formaldehyde in the presence of a suitable base (e.g. triethylamine) and a suitable solvent (e.g. N,N-dimethylformamide) according to procedures know to the person skilled in the art.

Likewise, the compound of formula II (i.e. 4-chloro-N-[2-[(4-chlorophenyl)methyl]-3-oxo-1,2,4-thiadiazol-5-yl]benzamide; referred to in the examples as Compound 1) may be prepared in accordance with techniques that are well known to those skilled in the art, e.g. the techniques described in international patent application no. WO 2011/004162.

The person skilled in the art will recognise that the compound of formula V may be obtained by the synthetic procedures exemplified herein, from available starting materials using appropriate reagents and reaction conditions.

Compounds of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention.

### Pharmaceutical Formulations

As indicated herein, the compounds of the invention are useful as therapeutic agents for treating a variety of medical disorders or conditions. Typically, compounds of the invention will be administered to a subject in need thereof in the form of a pharmaceutical formulation.

According to a second aspect of the invention, there is provided a pharmaceutical formulation comprising the compound of formula I (or a pharmaceutically acceptable salt or solvate thereof). Such formulations are referred to herein as the formulations of the invention. All embodiments and particular features thereof described herein in respect of the first aspect of the invention are disclosed herein in respect of the second aspect of the invention.

The pharmaceutical formulations of the second aspect of the invention may be prepared in accordance with standard and/or accepted pharmaceutical practice.

In an embodiment of the second aspect of the invention, the compound of the invention (or the pharmaceutically acceptable salt or solvate thereof) is the sole active pharmaceutical ingredient present in the formulation. In a further embodiment of the second aspect of the invention, the compound of the invention (or a pharmaceutically acceptable salt or solvate thereof) is present in the formulation alongside one or more other active pharmaceutical ingredients, or may be administered as part of a combination therapy with one or more other active pharmaceutical ingredients.

The formulations of the second aspect of the invention will generally be provided as a mixture comprising the compound of the invention (or a pharmaceutically acceptable salt or solvate thereof) and one or more pharmaceutically acceptable excipients. The one or more pharmaceutically acceptable excipients may be selected with due regard to the intended route of administration in accordance with standard pharmaceutical practice. Such pharmaceutically acceptable excipients are preferably chemically inert to the active compound and preferably have no detrimental side effects or toxicity under the conditions of use. Suitable pharmaceutical formulations may be found in, for example, Remington The Science and Practice of Pharmacy, 19th ed., Mack Printing Company, Easton, Pennsylvania (1995). A brief review of methods of drug delivery may also be found in e.g. Langer, Science 249, 1527 (1990).

For example, formulations of the invention may contain a lubricant, a binder, a filler, a surfactant, a diluent, an anti-adherent, a coating, a flavouring, a colourant, a glidant, a preservative, a sweetener, a disintegrant, an adsorbent, a buffering agent, an antioxidant, a chelating agent, a dissolution enhancer, a dissolution retardant and/or a wetting agent.

Particular pharmaceutically acceptable excipients that may be mentioned include microcrystalline cellulose, lactose monohydrate, crospovidone, magnesium stearate, colloidal silicon dioxide, anhydrous lactose, dicalcium phosphate, mannitol, pregelatinised starch, hydroxypropyl cellulose, povidone, low-substituted hydroxypropyl cellulose, croscarmellose sodium, sodium starch glycolate, sodium stearyl fumarate, talc, hydroxypropyl methylcellulose, polysorbate 80, sodium lauryl sulphate, poloxamer 188, poloxamer 407, propylene glycol, titanium dioxide, hypromellose phthalate, hypromellose acetate succinate, methacrylic acid, methyl methacrylate copolymer, Opadry® I, Opadry® II.

In the preparation of a pharmaceutical formulation of the compounds of the invention for oral administration, the compounds of the invention may be mixed, either together or separately, with one or more of the pharmaceutical excipients listed above.

Mixtures of a compound of the invention and one or more pharmaceutically acceptable excipients may be processed into pellets or granules, or compressed into tablets. Thus, pharmaceutical formulations of the invention include formulations that are provided in the form of a tablet, mini-tablets, blocks, pellets, particles, granules or a powder for oral administration. Mixtures of a compound of the invention and one or more pharmaceutically acceptable excipients may also be provided in a form that is suitable for subcutaneous or intramuscular delivery, such as an injectable solution or a freeze-dried powder suitable for reconstitution in a suitable fluid prior to administration.

The skilled person will understand that formulations described herein may act systemically, and may therefore be administered accordingly using suitable techniques known to those skilled in the art. Formulations as described herein will normally be administered orally, subcutaneously or intramuscularly in a suitable pharmaceutically acceptable dosage form. The pharmaceutical formulation of the second aspect of the invention is preferably an oral pharmaceutical formulation, particularly in view of the enhanced stability under acidic conditions that has been observed for compounds of the invention.

Formulations of the invention may be prepared for oral administration in the form of a capsule. For example, capsules such as soft gelatin capsules may be prepared containing the compound of the invention alone (or a pharmaceutically acceptable salt or solvate thereof), optionally together with a suitable vehicle, e.g. vegetable oil, fat etc. Similarly, hard gelatin capsules may contain the compound of the invention alone (or a pharmaceutically acceptable salt or solvate thereof) alone, or in combination with solid powdered ingredients such as a disaccharide (e.g. lactose or saccharose), an alcohol sugar (e.g. sorbitol or mannitol), a vegetable starch (e.g. potato starch or corn starch), a polysaccharide (e.g. amylopectin or cellulose derivatives) or gelling agent (e.g. gelatin).

Particular pharmaceutical formulations of the invention include formulations that may be mentioned are those provided in the form of a capsule or a tablet, e.g. for oral administration.

Pharmaceutical formulations that may be mentioned include those in which the compound of the invention (or a pharmaceutically acceptable salt or solvate thereof) is present in a total amount that is at least 1% (or at least 10%, at least 30% or at least 50%) by weight of the formulation and up to 99% by weight of the formulation. The weight ratio of the compound of the invention (or a pharmaceutically acceptable salt or solvate thereof) to the totality of the components (i.e. the compound of the invention and all pharmaceutical excipients, e.g. adjuvants, diluents and carriers) of the pharmaceutical formulation is at least 1:99 (or at least 10:90, at least 30:70 or at least 50:50) and up to 99:1.

A “therapeutically effective amount”, an “effective amount” and a “dosage” as used herein refer to an amount of a compound of the invention (or a pharmaceutically acceptable salt or solvate thereof) that is sufficient to produce a desired clinical effect, which can be a therapeutic and/or beneficial effect. The effective amount or dosage will vary with the age or general condition of the subject (e.g. a human), the severity of the condition being treated, the particular agents administered, the duration of the treatment, the nature of any concurrent treatment, the pharmaceutically acceptable excipients used, and like factors within the knowledge and expertise of those skilled in the art. As appropriate, a “therapeutically effective amount”, “effective amount” or “dosage” in any individual case can be determined by one of skill in the art by reference to the pertinent texts and literature and/or by using routine experimentation. Those skilled in the art will appreciate that the therapeutic effects need not be complete or curative, as long as some benefit is provided to the subject.

The skilled person will understand that compounds of the invention, and formulations thereof, may be administered (for example, by way of one or more preparations as described herein) at varying doses, with suitable doses being readily determined by one of skill in the art. The total dosage of the compound of the invention (or a pharmaceutically acceptable salt or solvate thereof) that is to be administered to a subject in need thereof may range from about 1 milligram per day (mg/day) to about 3000 mg/day, about 2 mg/day to about 2000 mg/day, or about 5 mg/day to about 1000 mg/day (e.g. from about 10 mg/day to about 500 mg/day). Such dosages may be, for example, oral dosages of the formulations of the second aspect of the invention. When the compound of the invention (or a pharmaceutically acceptable salt or solvate thereof) is to be administered either intramuscularly or subcutaneously, the skilled person will recognise that the dosage should be adjusted accordingly.

When administered orally, treatment with such formulations may comprise administration of a unit dose formulation containing from about 1 mg to about 3000 mg of the compound of the invention, for example from about 2 mg to about 2000 mg, or from about 5 mg to about 1000 mg (e.g. from about 10 mg to about 500 mg), of the compound of the invention. Advantageously, treatment may comprise administration of the compound of the invention (e.g. In the form of one or more capsules containing said formulation) using a single daily dose. Alternatively, the total daily dosage of the compound of the invention may be administered in divided doses two, three or four times daily (e.g. twice daily with reference to the doses described herein, such as a dose of 100 mg, 250 mg, 500 mg or 1000 mg twice daily). The skilled physician will recognise that the dosage will vary from subject to subject.

In particular embodiments, the daily dose of the compound of the invention administered to a subject is in the range of from about 1 to about 3000 mg, preferably from about 1 to about 1000 mg.

The term “about” as used herein when referring to a measurable value such as an amount of a compound, dose, time, temperature, and the like, refers to variations of 20%, 10%, 5%, 1%, 0.5%, or even 0.1% of the specified amount. It is contemplated that, at each instance, such terms may be replaced with the notation “±10%”, or the like (or by indicating a variance of a specific amount calculated based on the relevant value). It is also contemplated that, at each instance, such terms may be deleted.

For the avoidance of doubt, the dose administered to a subject, particularly a human subject, in the context of the present invention should be sufficient to effect a therapeutic response in the subject over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the subject to be treated, and the stage/severity of the disease.

In any event, the medical practitioner, or other skilled person, will be able to determine routinely the actual dosage which will be most suitable for an individual subject. The above-mentioned dosages are exemplary of the average case; there can, of course, be individual instances where higher or lower dosage ranges are merited, and such are within the scope of this invention.

### Medical Uses

As indicated herein, the compounds of the invention are useful as pharmaceuticals. Compounds of the invention are useful because they possess pharmacological activity, and/or are metabolised in the body following oral or parenteral administration to form a compound that possesses pharmacological activity.

Thus, according to a third aspect of the invention there is provided a compound of the invention, as hereinbefore defined (i.e. a compound as defined in the first aspect of the invention), or a pharmaceutical formulation as defined in respect of the second aspect of the invention, for use in medicine. For the avoidance of doubt, references to compounds as defined in the first aspect of the invention include references to compounds of formula I (including all embodiments thereof) and pharmaceutically acceptable salts and solvates thereof.

Compounds of the invention (i.e. a compound as defined in the first aspect of the invention), and formulations containing the same, may be particularly useful in treating a disorder or condition ameliorated by the activation of AMP-activated protein kinase (AMPK). Thus, in a fourth aspect of the invention, there is provided a compound of the invention, or a formulation comprising said compound, for use in the treatment of a disorder or condition ameliorated by the activation of AMPK.

Similarly, there is provided the use of a compound of the invention, or a formulation comprising said compound, in the manufacture of a medicament for the treatment of a disorder or condition ameliorated by the activation of AMPK. In a further alternative fourth aspect of the invention, there is provided a method of treating a disorder or condition ameliorated by the activation of AMPK comprising administering a compound of the invention (or a formulation comprising said compound) to a subject (e.g. a human) in need thereof.

By ‘activate AMPK’, we mean that the steady state level of phosphorylation of the Thr-172 moiety of the AMPK-α (AMPK-alpha) subunit is increased compared to the steady state level of phosphorylation in the absence of a compound of formula I or an active metabolite thereof (e.g. 4-chloro-N-[2-[(4-chlorophenyl)methyl]-3-oxo-1,2,4-thiadiazol-5-yl]benzamide). Alternatively, or in addition, we mean that there is a higher steady state level of phosphorylation of any other proteins downstream of AMPK, such as acetyl-CoA carboxylase (ACC).

The terms “disorder or condition ameliorated by the activation of AMPK” will be understood by those skilled in the art to include cardiovascular disease (such as heart failure), diabetic kidney disease, diabetes (such as type 2 diabetes), insulin resistance, non-alcoholic fatty liver disease, non-alcoholic steatohepatitis, pain, opioid addiction, obesity, cancer, inflammation (including chronic inflammatory diseases), autoimmune diseases, osteoporosis, intestinal diseases and hyperinsulinemia associated with obesity or cardiovascular disease. Other diseases or conditions that may be ameliorated by the activation of AMPK include hyperinsulinemia and associated conditions, a condition/disorder where fibrosis plays a role, sexual dysfunction and neurodegenerative diseases.

The term “cancer” will be understood by those skilled in the art to include one or more diseases in the class of disorders that is characterized by uncontrolled division of cells and the ability of these cells to invade other tissues, either by direct growth into adjacent tissue through invasion, proliferation or by implantation into distant sites by metastasis. By “proliferation” we include an increase in the number and/or size of cancer cells. By “metastasis” we mean the movement or migration (e.g. Invasiveness) of cancer cells from a primary tumor site in the body of a subject to one or more other areas within the subject's body (where the cells can then form secondary tumors).

Thus, compounds of the invention may be suitable for use in the treatment of any cancer type, including all tumors (non-solid and, preferably, solid tumors, such as carcinoma, adenoma, adenocarcinoma, blood cancer, irrespective of the organ). For example, the cancer cells may be selected from the group consisting of cancer cells of the breast, bile duct, brain, colon, stomach, reproductive organs, thyroid, hematopoietic system, lung and airways, skin, gallbladder, liver, nasopharynx, nerve cells, kidney, prostate, lymph glands and gastrointestinal tract. Preferably, the cancer is selected from the group consisting of colon cancer (including colorectal adenomas), breast cancer (e.g. postmenopausal breast cancer), endometrial cancer, cancers of the hematopoietic system (e.g. leukemia, lymphoma, etc.), thyroid cancer, kidney cancer, oesophageal adenocarcinoma, ovarian cancer, prostate cancer, pancreatic cancer, gallbladder cancer, liver cancer and cervical cancer. More preferably, the cancer is selected from the group consisting of colon, prostate and, particularly, breast cancer. Where the cancer is a non-solid tumor, it is preferably a hematopoietic tumor such as a leukemia (e.g. Acute Myelogenous Leukemia (AML), Chronic Myelogenous Leukemia (CML), Acute Lymphocytic Leukemia (ALL), or Chronic Lymphocytic Leukemia (CLL). Preferably the cancer cells are breast cancer cells.

The term “diabetes” (i.e. diabetes mellitus) will be understood by those skilled in the art to refer to both type 1 (insulin-dependent) diabetes and type 2 (insulin-independent) diabetes, both of which involve the malfunction of glucose homeostasis. The compounds of the invention and formulations thereof may be particularly suitable for use in the treatment of type 1 diabetes and/or type 2 diabetes. The compounds of the invention are particularly suited for the treatment of type 2 diabetes.

As well as being useful in the treatment of diabetes, the compounds of the invention are also suitable for treating diabetic kidney disease (i.e. diabetic nephropathy). “Diabetic kidney disease” refers to kidney damage caused by diabetes and is a serious complication of type 1 diabetes and type 2 diabetes. Diabetic kidney disease affects the kidneys' ability to remove waste products from blood to be excreted as urine, and can lead to kidney failure.

Moreover, the compounds of the invention are also suitable for treating chronic kidney disease, including chronic kidney disease in the absence of type 2 diabetes. “Chronic kidney disease” is a condition characterised by a gradual loss of kidney function over time. Chronic kidney disease usually occurs as a result of one or more other diseases or conditions that affect the kidneys, such as high blood pressure, diabetes, high cholesterol, kidney infections, glomerulonephritis, polycystic kidney disease, obstruction of the urinary tract blockages in the flow of urine and long-term medication use.

The term “hyperinsulinemia or an associated condition” will be understood by those skilled in the art to include hyperinsulinemia, type 2 diabetes, glucose intolerance, insulin resistance, metabolic syndrome, dyslipidemia, hyperinsulinism in childhood, hypercholesterolemia, high blood pressure, obesity, fatty liver conditions, diabetic nephropathy, diabetic neuropathy, diabetic retinopathy, cardiovascular disease, atherosclerosis, cerebrovascular conditions such as stroke, systemic lupus erythematosus, neurodegenerative diseases such as Alzheimer's disease, and polycystic ovary syndrome. Other disease states include progressive renal disease such as chronic renal failure.

In particular, compounds of the invention and formulations thereof may be suitable for use in the treatment of obesity associated with hyperinsulinemia and/or cardiovascular disease associated with hyperinsulinemia.

Compounds of the invention and formulations thereof may also be suitable for use in the treatment of cardiovascular disease, such as heart failure, wherein said cardiovascular disease is not associated with hyperinsulinemia. Similarly, compounds of the invention and formulations thereof may also be suitable for use in the treatment of obesity which is not associated with hyperinsulinemia. For the avoidance of doubt, the treatment of obesity and/or cardiovascular disease (such as heart failure) where AMPK activation may be beneficial is included within the scope of the invention.

A condition/disorder where fibrosis plays a role includes (but is not limited to) scar healing, keloids, scleroderma, pulmonary fibrosis (including idiopathic pulmonary fibrosis), nephrogenic systemic fibrosis, and cardiovascular fibrosis (including endomyocardial fibrosis), systemic sclerosis, liver cirrhosis, eye macular degeneration, retinal and vitreal retinopathy, Crohn's/inflammatory bowel disease, post-surgical scar tissue formation, radiation and chemotherapeutic-drug induced fibrosis, and cardiovascular fibrosis.

The compounds of invention may also be useful in the treatment of sexual dysfunction (e.g. the treatment of erectile dysfunction). The compounds of invention may also be useful in the treatment of inflammation.

Neurodegenerative diseases that may be mentioned include Alzheimer's disease, Parkinson's disease and Huntington's disease, amyotrophic lateral sclerosis, polyglutamine disorders, such as spinal and bulbar muscular atrophy (SBMA), dentatorubral and pallidoluysian atrophy (DRPLA), and a number of spinocerebellar ataxias (SCA).

Compounds of the invention may be useful in the treatment of a non-alcoholic fatty liver disease (NAFLD).

Non-alcoholic fatty liver disease (NAFLD) is defined by excessive fat accumulation in the form of triglycerides (steatosis) in the liver (designated as an accumulation of greater than 5% of hepatocytes histologically). It is the most common liver disorder in developed countries (for example, affecting around 30% of US adults) and most patients are asymptomatic. If left untreated, the condition may progressively worsen and may ultimately lead to cirrhosis of the liver. NAFLD is particularly prevalent in obese patients, with around 80% thought to have the disease.

NAFLD may be diagnosed wherein alcohol consumption of the patient is not considered to be a main causative factor. A typical threshold for diagnosing a fatty liver disease as “not alcohol related” is a daily consumption of less than 20 g for female subjects and less than 30 g for male subjects.

Particular diseases or conditions that are associated with NAFLD include metabolic conditions such as diabetes, hypertension, obesity, dyslipidemia, abetalipoproteinemia, glycogen storage diseases, Weber-Christian disease, acute fatty liver of pregnancy, and lipodystrophy. Other non-alcohol related factors related to fatty liver diseases include malnutrition, total parenteral nutrition, severe weight loss, refeeding syndrome, jejunoileal bypass, gastric bypass, polycystic ovary syndrome and diverticulosis.

Non-alcoholic steatohepatitis (NASH) is the most aggressive form of NAFLD, and is a condition in which excessive fat accumulation (steatosis) is accompanied by inflammation of the liver. If advanced, NASH can lead to the development of scar tissue in the liver (fibrosis) and, eventually, cirrhosis. As described above, the compounds of the invention have been found to be useful in the treatment of NAFLD and inflammation. It follows that the compounds of the invention are also useful in the treatment of NASH. Therefore, in a further embodiment, the treatment is of non-alcoholic steatohepatitis (NASH).

It has been shown that AMPK activator compounds (such as 4-chloro-N-[2-[(4-chlorophenyl)methyl]-3-oxo-1,2,4-thiadiazol-5-yl]benzamide (i.e. the compound of formula II)) are capable of treating pain (Das V, et al. Reg Anesth Pain Med 2019; 0:1-5. doi:10.1136/rapm-2019-100839 and Das V, et al. Reg Anesth Pain Med 2019; 0:1-6. doi:10.1136/rapm-2019-100651) and such compounds may be considered to be analgesics. It therefore follows that, since the compounds of the invention are capable of activating AMPK, or are metabolised in vivo to form a known AMPK activator compound, the compounds of the invention may be useful in the treatment of pain. In particular, the compounds of the invention may be useful in the treatment of patients with severe pain, chronic pain or useful in the management of pain after surgery.

Opioid-based therapies, such as opioid analgesics, are used to treat severe, chronic cancer pain, acute pain (e.g. during recovery from surgery and breakthrough pain) and their use is increasing in the management of chronic, non-malignant pain. However, the increasing use of opioid-based therapies to treat pain has resulted in an increase of opioid dependence (e.g. opioid addiction). As AMPK activators, the compounds of the invention may be used to treat pain in place of an opioid-based therapy, as known by those skilled in the art. Accordingly, the compounds of the invention may be useful in treating opioid addiction.

Particular autoimmune diseases know to those skilled in the art include Crohn's/inflammatory bowel disease, systemic lupus erythematosus and type 1 diabetes.

Particular intestinal diseases that should be mentioned include Crohn's/inflammatory bowel disease and cancer of gastrointestinal tract.

The skilled person will understand that references to the “treatment” of a particular condition (or, similarly, to “treating” that condition) will take their normal meanings in the field of medicine. In particular, the terms may refer to achieving a reduction in the severity and/or frequency of occurrence of one or more clinical symptom associated with the condition, as judged by a physician attending a subject having or being susceptible to such symptoms.

The skilled person will understand that such treatment or prevention will be performed in a subject in need thereof. The need of a subject for such treatment or prevention may be assessed by those skilled the art using routine techniques. In the context of the present invention, a “subject in need” of the compound of the invention includes a subject that is suffering a disorder or condition ameliorated by the activation of AMPK. As used herein, the terms “disease” and “disorder” (and, similarly, the terms condition, illness, medical problem, and the like) may be used interchangeably.

Without wishing to be bound by theory, it is believed that the administration of a compound of the invention enhances the bioavailability of 4-chloro-N-[2-[(4-chlorophenyl)methyl]-3-oxo-1,2,4-thiadiazol-5-yl]benzamide in the systemic circulation. Administration of formulations comprising a compound of the invention has been shown to provide an approximately ten-fold increase in the plasma concentration of 4-chloro-N-[2-[(4-chlorophenyl)methyl]-3-oxo-1,2,4-thiadiazol-5-yl]benzamide under certain circumstances compared to administration of a formulation that comprises 4-chloro-N-[2-[(4-chlorophenyl)methyl]-3-oxo-1,2,4-thiadiazol-5-yl]benzamide.

Compounds of the invention (and formulations thereof) may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, other therapies known in the prior art, whether for use in the above-stated indications or otherwise. In particular, compounds of the invention may have the advantage that they are more efficacious and/or exhibit advantageous properties in vivo.

### FIGURES

The following drawings are provided to illustrate various aspects of the present inventive concept and are not intended to limit the scope of the present invention unless specified herein.

FIG. 1 shows western blot images that demonstrate that Compound 3 increases phosphorylation of AMPK in a dose-dependent manner.

FIG. 2 shows comparative results of oral pharmacokinetic studies using Compound 1 and Compound 3.

FIG. 3 shows comparative results of oral pharmacokinetic studies using Compounds 5, 6, 7, 9, 10, 13, 14, 16 and 17.

### EXAMPLES

The present invention is explained in greater detail in the following non-limiting examples.

The reaction schemes described below are intended to provide a general description of the methodology employed in the preparation of the compounds of the invention. The examples provided herein are offered to illustrate but not limit the compounds of the invention, as well as the preparation of such compounds and intermediates.

All starting materials, building blocks, reagents, acids, bases, dehydrating agents, solvents and catalysts utilised to synthesise the compounds of the invention are either commercially available or can be routinely prepared by procedures described in the literature, for example, Houben-Weyl “Science of Synthesis” volumes 1-48, Georg Thieme Verlag, and subsequent versions thereof.

A reaction may be carried out in the presence of a suitable solvent or diluent or of mixture thereof in a manner known to those skilled in the art of organic synthesis. A reaction may also be carried out, if needed, in the presence of an acid or a base, with cooling or heating, for example in a temperature range from about −30° C. to about 150° C. In some embodiments, a reaction is carried out in a temperature range from about 0° C. to about 100° C., and more particularly, in a temperature range from room temperature to about 80° C., in an open or closed reaction vessel and/or in the atmosphere of an inert gas, for example nitrogen.

### Abbreviations

Abbreviations as used herein will be known to those skilled in the art. In particular, the following abbreviations may be used herein.
AUC: Area under the concentration-time curve
aq: Aqueous
b.w.: Body weight
Cₘₐₓ: Peak plasma concentration
d: Doublet
DCC: N,N′-dicyclohexylcarbodiimide
DCM: Dichloromethane
DMAP: 4-Dimethylaminopyridine
DMSO: Dimethylsulfoxide
eq.: equivalent
ESI: Electrospray ionisation
Et₃N: Triethylamine
EtOH: Ethanol
g: Gram
h: Hours
HPLC: High performance liquid chromatography
LC: Liquid chromatography
LCMS: Liquid chromatography—mass spectrometry
LC-MS/MS: Liquid chromatography—(tandem) mass spectrometry
LLOQ: Lower limit of quantification
m: Multiplet
MeOH: Methanol
MeOD: Methanol-d₄
Min: Minutes
mL: Millilitre
MRT: Mean residence time
ND: Not detected
NMR: Nuclear magnetic resonance
RT: Room temperature
s: Singlet
T_{1/2}: Half-life
TBDMS-Cl: tert-Butyldimethylsilyl chloride
Tₘₐₓ: Time to reach the peak plasma concentration
THF: Tetrahydrofuran

### Instrumentation Conditions

### LC Parameters

Column: Agilent Zorbax SB-C8, 50×4.6 mm, 350 μm
Mobile phase: 5 mM ammonium acetate:methanol with 0.1% formic acid (15:85 v/v)
Separation mode: Isocratic
Flow rate: 0.800 mL/min
Injection volume: 2 μL
Auto sampler temperature: 4° C.
Column oven temperature: 40° C.
LC-MS/MS Shimadzu LCMS-8045
Source ESI
Polarity Positive
m/z of analyte (Compound 1) 379.800>89.100
m/z of internal standard (warfarin) 309.100>251.100

The present invention will be further described by reference to the following examples, which are not intended to limit the scope of the invention.

### Example 1: Preparation of Dimethylamino-Acetic Acid 5-[(Z)-4-chloro-benzoylimino]-2-(4-chloro-benzyl)-3-oxo-[1,2,4]thiadiazolidin-4-ylmethyl ester (Compound 3)

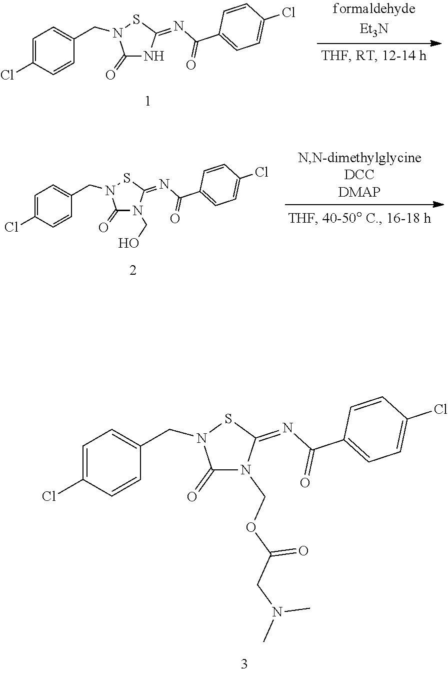 [SMILES: O=C(/N=c1\[nH]c(=O)n(Cc2ccc(Cl)cc2)s1)c1ccc(Cl)cc1 ; O=C(/N=c1/sn(Cc2ccc(Cl)cc2)c(=O)n1CO)c1ccc(Cl)cc1 ; CN(C)CC(=O)OCn1c(=O)n(Cc2ccc(Cl)cc2)s/c1=N/C(=O)c1ccc(Cl)cc1]

### Compound 1: 4-chloro-N-[2-[(4-chlorophenyl)methyl]-3-oxo-1,2,4-thiadiazol-5-yl]benzamide

Compound 1 was prepared in accordance with the procedures described in WO 2011/004162.

### Compound 2: 4-chloro-N-{2-[(4-chlorophenyl)methyl]-4-(hydroxymethyl)-3-oxo-1,2,4-thiadiazolidin-5-ylidene}benzamide

To a stirring solution of Compound 1 (100 g, 0.26 mol) in THF (2.0 L) were added Et₃N (150 mL, 1.05 mol) followed by 37% formaldehyde (85 mL, 1.05 mol). The reaction mixture was stirred at RT for 12 to 14 h.

The reaction mixture was then concentrated to remove THF. The crude material obtained was treated with toluene (2×100 mL) and dried under reduced pressure at 35 to 40° C. to yield Compound 2 as white solid (103 g, 95.5% yield).

### Compound 3: Dimethylamino-Acetic Acid 5-[(Z)-4-chloro-benzoylimino]-2-(4-chloro-benzyl)-3-oxo-[1,2,4]thiadiazolidin-4-ylmethyl Ester

To a stirring solution of N,N-dimethylglycine (50.3 g, 0.48 mol) in THF (2.0 L) were added DMAP (8.93 g, 0.07 mol) and DCC (100.6 g, 0.48 mol). After 10 minutes, Compound 2 (200 g, 0.49 mol) was added to the reaction mixture in four batches over 30 minutes. The reaction mixture was stirred at 40 to 50° C. for 16 to 18 h. The reaction mixture was cooled to 20 to 30° C., filtered through Celite®, washing with THF (200 mL), and the filtrate was concentrated to remove THF. The crude material obtained was slurried with MeOH (300 mL) for 30 min at RT and filtered, washing with MeOH (50 mL). The collected solid was dried under reduced pressure at 35 to 40° C. to afford Compound 3 as a white solid (102 g, 42% yield).

¹H NMR (400 MHz, DMSO) δ (ppm): 2.20 (s, 6H), 3.21 (s, 2H), 4.84 (s, 2H), 6.03 (s, 2H), 7.40 (dd, 4H), 7.63 (d, 2H), 8.15 (d, 2H).

LCMS: 495.2 [M+H].

HPLC: 96.3% purity at 8.57 minutes.

### Example 2: Preparation of Citric Acid Salt of Dimethylamino-Acetic Acid 5-[(Z)-4-chloro-benzoylimino]-2-(4-chloro-benzyl)-3-oxo-[1,2,4]thiadiazolidin-4-ylmethyl Ester (Compound 4)

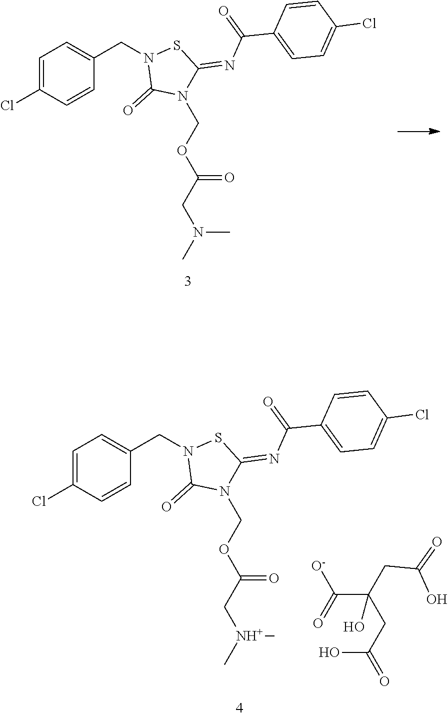 [SMILES: CN(C)CC(=O)OCn1c(=O)n(Cc2ccc(Cl)cc2)s/c1=N\C(=O)c1ccc(Cl)cc1 ; C[NH+](C)CC(=O)OCn1c(=O)n(Cc2ccc(Cl)cc2)s/c1=N\C(=O)c1ccc(Cl)cc1 ; O=C(O)CC(O)(CC(=O)O)C(=O)[O-]]

To a stirring solution of dimethylamino-acetic acid 5-[(Z)-4-chloro-benzoylimino]-2-(4-chloro-benzyl)-3-oxo-[1,2,4]thiadiazolidin-4-ylmethyl ester (Compound 3; 550 mg, 1.1 mmol) in acetone (30.0 v) was added citric acid (0.9 eq.). The reaction was stirred at RT for 2.3 h. The precipitated compound was then collected by filtration and dried to afford Compound 4 (450 mg).

¹H NMR (400 MHz, DMSO) δ (ppm): 2.20 (s, 6H), 3.21 (s, 2H), 4.84 (s, 2H), 6.03 (s, 2H), 7.40 (dd, 4H), 7.63 (d, 2H), 8.15 (d, 2H).

LCMS: 495.2 [M+H].

HPLC: 98.3% purity at 9.95 minutes

### Example 3: Preparation of 2-({[5-(4-chlorobenzamido)-2-[(4-chlorophenyl)methyl]-3-oxo-1,2,4-thiadiazolidin-4-yl]methoxy}carbonyl)pyrrolidin-1-ium Trifluoroacetate (Compound 5)

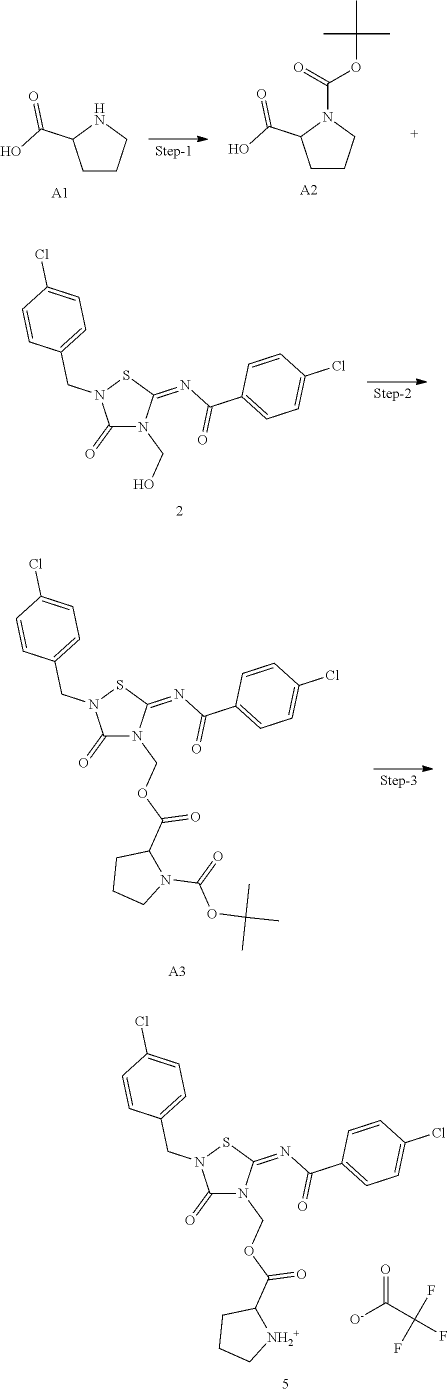

### Step-1: 1-[(tert-butoxy)carbonyl]pyrrolidine-2-carboxylic Acid (Compound A2)

To a suspension of L-proline (A1) (1.0 g, 8.68 mmol) in DCM (10 mL), was added triethylamine (1.34 mL, 9.55 mmol) at 0° C. followed by di-tert-butyl dicarbonate (2.18 mL, 9.55 mmol) and the resulting reaction mixture was stirred for 16 h at RT. The reaction mixture was carefully acidified with 5% citric acid solution to pH 3. The product was extracted with ethyl acetate, and the extract was dried and evaporated in a vacuum to afford Compound A2 as a white solid (1.55 g, 83% yield).

### Step-2: 1-tert-butyl 2-[5-(4-chlorobenzamido)-2-[(4-chlorophenyl)methyl]-3-oxo-1,2,4-thiadiazolidin-4-yl]methyl pyrrolidine-1,2-dicarboxylate (Compound A3)

To a stirring solution of Compound A2 (0.55 g, 2.56 mmol) in THF (11 mL, 20V) was added DMAP (0.06 g, 0.49 mmol) followed by DCC (0.52 g, 2.56 mmol) at 0-5° C. and stirred for 20 min. To the reaction mixture Compound 2 (0.7 g, 1.7 mmol) was added at 0-5° C. and the reaction mixture was stirred for 16 h at ambient temperature (25-30° C.). The reaction mixture was filtered, and the filtrate was concentrated and purified by silica gel column chromatography to afford Compound A3 as a white solid (0.5 g, 50% yield).

### Step-3: 2-({[5-(4-chlorobenzamido)-2-[(4-chlorophenyl)methyl]-3-oxo-1,2,4-thiadiazolidin-4-yl]methoxy}carbonyl)pyrrolidin-1-ium Trifluoroacetate (Compound 5)

To a stirring solution of Compound A3 (0.5 g, 0.82 mmol) in dichloromethane (5 mL) was added trifluoroacetic acid (1 mL, 2.0 vol) at 0-5° C. The reaction mixture was stirred for 16 h at ambient temperature (25-30° C.). The reaction mixture was concentrated to remove the dichloromethane. The residue obtained was re-precipitated with methanol/ether and filtered under vacuum to afford Compound 5 as a white solid (0.2 g, 39% yield).

¹H NMR (400 MHz, DMSO) δ (ppm): 1.90 (m, 2H), 1.98 (m, 1H), 2.22 (m, 1H), 3.18 (m, 2H), 4.48 (m, 1H), 4.84 (s, 2H), 6.13 (s, 2H), 7.42 (dd, 4H), 7.63 (d, 2H), 8.16 (d, 2H).

LCMS: 507.3 [M+H].

HPLC: 95.3% purity at 10.07 minutes.

In Step 1 of this example, L-proline was used, i.e. the naturally occurring L-enantiomer of the amino acid. In all subsequent examples, the naturally occurring L-enantiomer of the respective amino acids (or alkylated and/or protected derivatives thereof) were used.

### Example 4: Preparation of 6-{[5-(4-chlorobenzamido)-2-[(4-chlorophenyl)methyl]-3-oxo-1,2,4-thiadiazolidin-4-yl]methoxy}-6-oxohexane-1,5-bis(aminium) di-trifluoroacetate (Compound 6)

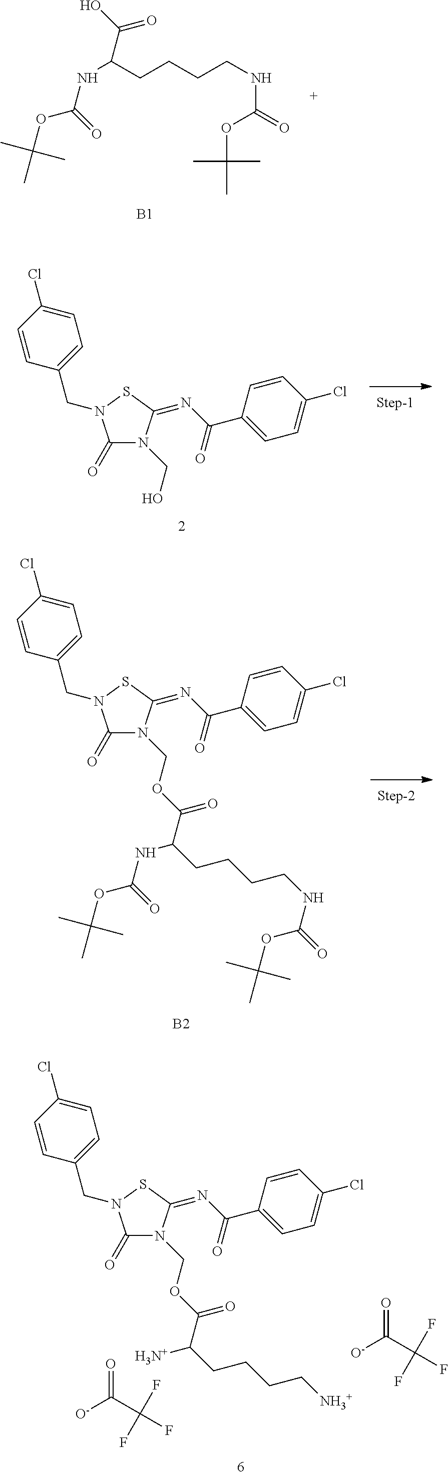

### Step 1: [5-(4-chlorobenzamido)-2-[(4-chlorophenyl)methyl]-3-oxo-1,2,4-thiadiazolidin-4-yl]methyl 2,6-bis({[(tert-butoxy)carbonyl]amino})hexanoate (Compound B2)

To a stirring solution of 2,6-bis({[(tert-butoxy)carbonyl]amino})hexanoic acid (Compound B1; 0.84 g, 2.43 mmol) in THF (17 mL, 20V) was added DMAP (0.074 g, 0.60 mmol) followed by DCC (0.50 g, 2.43 mmol) at 0-5° C. and stirred for 20 minutes. To the reaction mixture Compound 2 (0.5 g, 1.21 mmol) was added at 0-5° C. and the reaction mixture was stirred for 16 h at ambient temperature (25-30° C.). The reaction mixture was filtered, and the filtrate was concentrated and purified by silica gel column chromatography to afford Compound B2 as a white solid (0.5 g, 55% yield).

### Step-2: 6-{[5-(4-chlorobenzamido)-2-[(4-chlorophenyl)methyl]-3-oxo-1,2,4-thiadiazolidin-4-yl]methoxy}-6-oxohexane-1,5-bis(aminium) di-trifluoroacetate (6)

To a stirring solution of Compound B2 (0.5 g, 0.67 mmol) in dichloromethane (5 mL) was added trifluoroacetic acid (1 mL, 2.0 vol) at 0-5° C. The reaction mixture was stirred for 16 h at ambient temperature (25-30° C.). The reaction mixture was concentrated to remove the dichloromethane. The residue obtained was re-precipitated with acetone/hexanes and filtered under vacuum to afford Compound 6 as a white solid (0.28 g, 63% yield).

¹H NMR (400 MHz, DMSO) δ (ppm): 1.40 (m, 4H), 1.75 (m, 2H), 2.62 (m, 2H), 4.05 (m, 1H), 4.85 (s, 2H), 6.13 (s, 2H), 7.42 (dd, 4H), 7.63 (d, 2H), 8.16 (d, 2H).

LCMS: 538.3 [M+H].

HPLC: 95.0% purity at 8.28 minutes.

### Example 5: Preparation of (6-azaniumyl-1-{[5-(4-chlorobenzamido)-2-[(4-chlorophenyl)methyl]-3-oxo-1,2,4-thiadiazolidin-4-yl]methoxy}-1-oxohexan-2-yl)dimethylazanium di-trifluoroacetate (Compound 7)

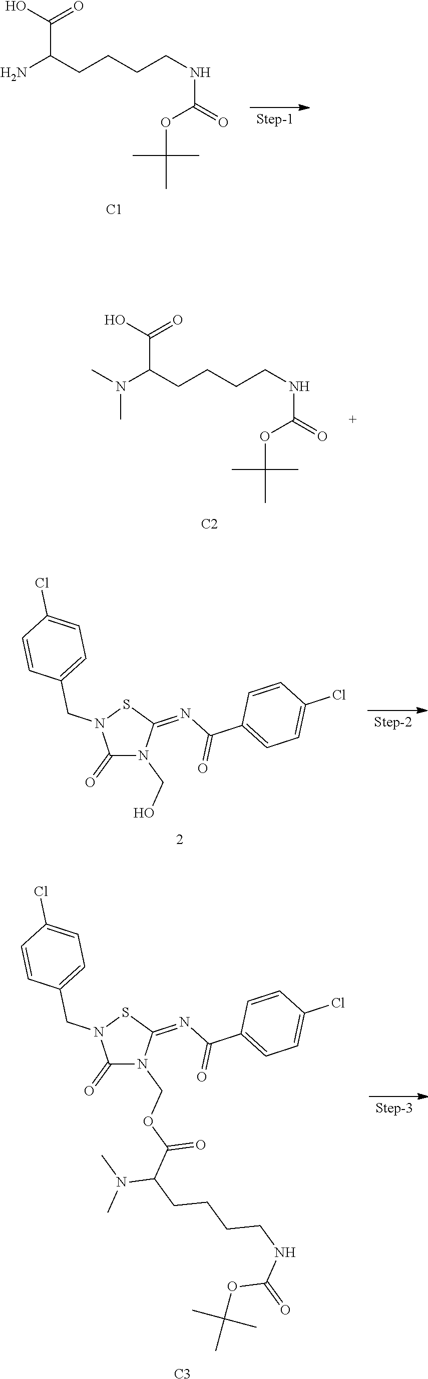

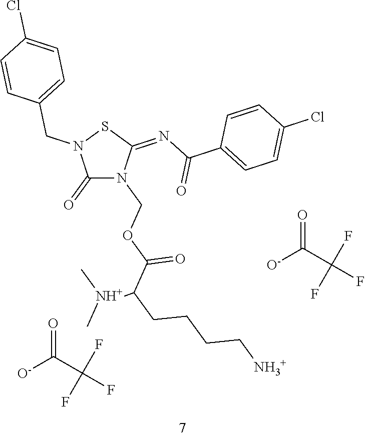 [SMILES: C[NH+](C)C(CCCC[NH3+])C(=O)OCn1c(=O)n(Cc2ccc(Cl)cc2)s/c1=N/C(=O)c1ccc(Cl)cc1 ; O=C([O-])C(F)(F)F ; O=C([O-])C(F)(F)F]

### Step-1: 6-{[(tert-butoxy)carbonyl]amino}-2-(dimethylamino)hexanoic Acid (Compound C2)

To a solution of Boc-Lys-OH (CM) (2.0 g, 8.1 mmol) in methanol (20 mL), were added 37% formaldehyde (2.6 mL, 32.4 mmol) followed by Pd/C (0.2 g, 10% w/w) and the resulting mixture was hydrogenated in a parr apparatus (5 kg, H₂ pressure) for 16 h. After completion of the reaction, the reaction mixture was filtered through Celite® and concentrated under reduced pressure. The residue was triturated with ether to afford Compound C2 as a white solid (1.5 g, 67% yield).

### Step-2: [5-(4-chlorobenzamido)-2-[(4-chlorophenyl)methyl]-3-oxo-1,2,4-thiadiazolidin-4-yl]methyl 6-{[(tert-butoxy)carbonyl]amino}-2-(dimethyl amino) hexanoate (Compound C3)

To a stirring solution of Compound C2 (0.66 g, 2.43 mmol) in THF (14 mL, 20V) was added DMAP (0.035 g, 0.29 mmol) followed by DCC (0.50 g, 2.43 mmol) at 0-5° C. and stirred for 20 min. To the reaction mixture Compound 2 (0.4 g, 0.97 mmol) was added at 0-5° C. and the reaction mixture was stirred for 16 h at ambient temperature (25-30° C.). The reaction mixture was filtered, and the filtrate was concentrated and purified by reverse phase column chromatography (8:2 acetonitrile and water) to afford Compound C3 as a white solid (0.18 g, 64.9% yield).

### Step-3: (6-azaniumyl-1-{[5-(4-chlorobenzamido)-2-[(4-chlorophenyl)methyl]-3-oxo-1,2,4-thiadiazolidin-4-yl]methoxy}-1-oxohexan-2-yl)dimethylazanium di-trifluoroacetate (Compound 7)

To a stirring solution of Compound C3 (0.18 g, 0.027 mmol) in dichloromethane (3.6 mL) was added trifluoroacetic acid (0.36 mL, 2.0 vol) at 0-5° C. The reaction mixture was stirred for 3 h at ambient temperature (25-30° C.). The reaction mixture was concentrated to remove the dichloromethane. The residue obtained was re-precipitated with acetone/hexanes and filtered under vacuum to afford Compound 7 as an off white solid (0.15 g, 81% yield).

¹H NMR (400 MHz, D₂O) δ (ppm): 1.24 (m, 2H), 1.39 (m, 2H), 1.80 (m, 2H), 2.73 (s, 6H), 4.77 (s, 2H), 6.16 (m, 2H), 7.35 (dd, 4H), 7.58 (d, 2H), 8.12 (d, 2H).

LCMS: 566.4 [M+H].

HPLC: 97.5% purity at 8.09 minutes.

### Example 6: Preparation of (2-{[5-(4-chlorobenzamido)-2-[(4-chlorophenyl)methyl]-3-oxo-1,2,4-thiadiazolidin-4-yl]methoxy}-2-oxoethyl)(methyl)azanium Trifluoroacetate (Compound 8)

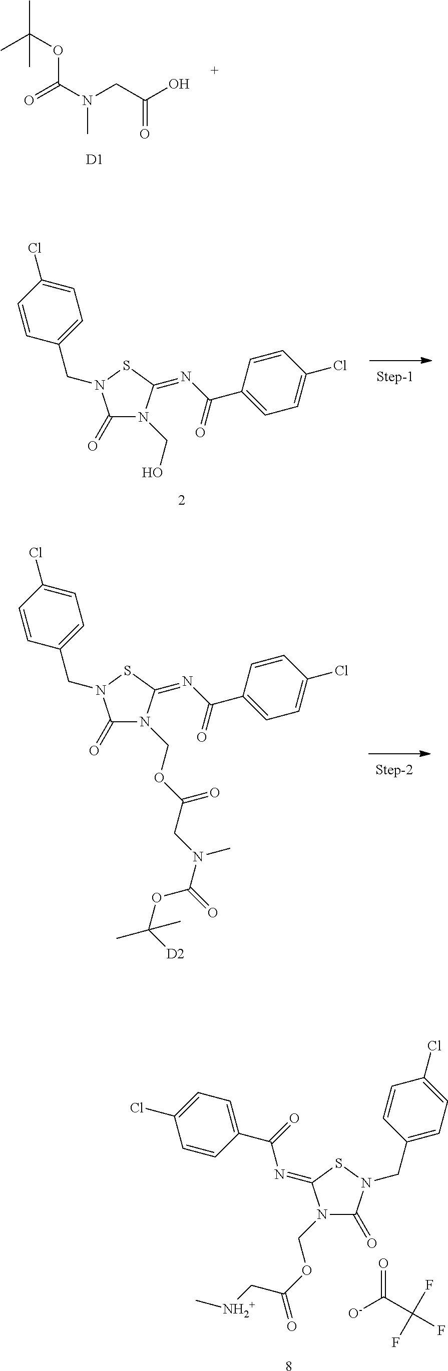

### Step-1: [5-(4-chlorobenzamido)-2-[(4-chlorophenyl)methyl]-3-oxo-1,2,4-thiadiazolidin-4-yl]methyl 2-{[(tert-butoxy)carbonyl](methyl)amino}acetate (D2)

To a stirring solution of N-Boc sarcosine (Compound D1; 0.57 g, 3.04 mmol) in THF (12 mL, 20V) was added DMAP (0.044 g, 0.36 mmol) followed by DCC (0.62 g, 3.04 mmol) at 0-5° C. and stirred for 20 min. To the reaction mixture Compound 2 (0.5 g, 1.21 mmol) was added at 0-5° C. and the reaction mixture was stirred for 16 h at ambient temperature (25-30° C.). The reaction mixture was filtered, and the filtrate was concentrated and purified by reverse phase column chromatography to afford Compound D2 as a white solid (0.22 g, 31% yield).

### Step-2: (2-{[5-(4-chlorobenzamido)-2-[(4-chlorophenyl)methyl]-3-oxo-1,2,4-thiadiazolidin-4-yl]methoxy}-2-oxoethyl)(methyl)azanium Trifluoroacetate (Compound 8)

To a stirring solution of Compound D2 (0.2 g, 0.34 mmol) in dichloromethane (4.0 mL) was added trifluoroacetic acid (0.4 mL, 2.0 vol) at 0-5° C. The reaction mixture was stirred for 16 h at ambient temperature (25-30° C.). The reaction mixture was concentrated to remove the dichloromethane. The residue obtained was triturated with diethyl ether to afford Compound 8 as a white solid (0.17 g, 84% yield).

¹H NMR (400 MHz, DMSO) δ (ppm): 2.57 (s, 3H), 4.09 (s, 2H), 4.85 (s, 2H), 6.13 (m, 2H), 7.42 (dd, 4H), 7.64 (d, 2H), 8.17 (d, 2H).

LCMS: 481.3 [M+H].

HPLC: 98.6% purity at 8.91 minutes.

### Example 7: Preparation of 2-{[5-(4-chlorobenzamido)-2-[(4-chlorophenyl)methyl]-3-oxo-1,2,4-thiadiazolidin-4-yl]methoxy}-2-oxoethan-1-aminium Trifluoroacetate (Compound 9)

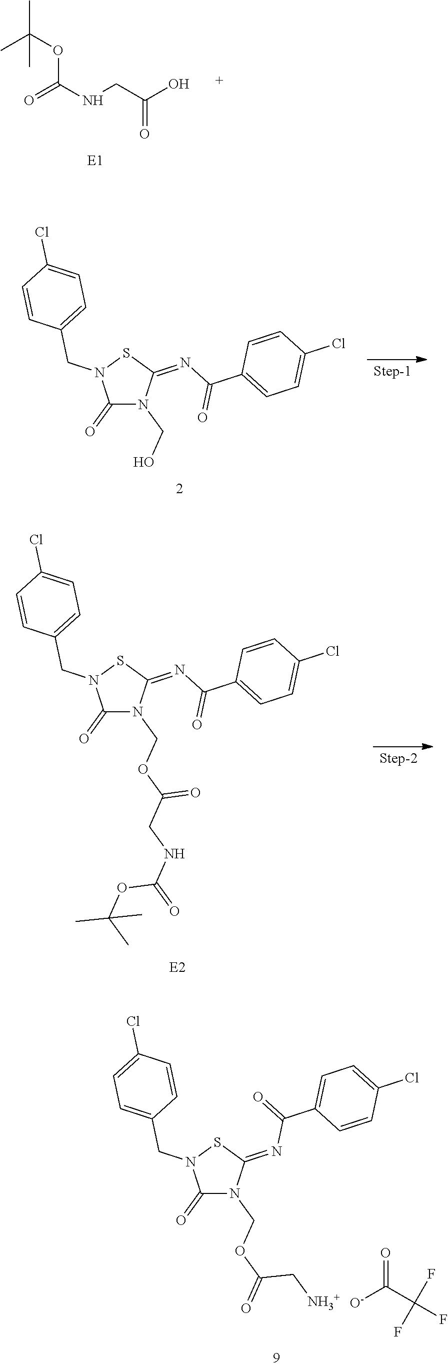 [SMILES: CC(C)(C)OC(=O)NCC(=O)O ; O=C(/N=c1/sn(Cc2ccc(Cl)cc2)c(=O)n1CO)c1ccc(Cl)cc1 ; CC(C)(C)OC(=O)NCC(=O)OCn1c(=O)n(Cc2ccc(Cl)cc2)s/c1=N/C(=O)c1ccc(Cl)cc1 ; "E2" ; [NH3+]CC(=O)OCn1c(=O)n(Cc2ccc(Cl)cc2)s/c1=N\C(=O)c1ccc(Cl)cc1 ; O=C([O-])C(F)(F)F]

### Step-1: [5-(4-chlorobenzamido)-2-[(4-chlorophenyl)methyl]-3-oxo-1,2,4-thiadiazolidin-4-yl]methyl 2-{[(tert-butoxy)carbonyl]amino}acetate (Compound E2)

To a stirring solution of N-Boc glycine (Compound E1; 0.53 g, 3.04 mmol) in THF (11 mL, 20V) was added DMAP (0.044 g, 0.36 mmol) followed by DCC (0.62 g, 3.04 mmol) at 0-5° C. and stirred for 20 min. To the reaction mixture, compound 2 (0.5 g, 1.21 mmol) was added at 0-5° C. and the reaction mixture was stirred for 16 h at ambient temperature (25-30° C.). The reaction mixture was filtered, and the filtrate was concentrated and purified by reverse phase column chromatography to afford Compound E2 as a white solid (0.32 g, 46% yield).

### Step-2: 2-{[5-(4-chlorobenzamido)-2-[(4-chlorophenyl)methyl]-3-oxo-1,2,4-thiadiazolidin-4-yl]methoxy}-2-oxoethan-1-aminium Trifluoroacetate (Compound 9)

To a stirring solution of Compound E2 (0.3 g, 0.52 mmol) in dichloromethane (3.0 mL) was added trifluoroacetic acid (0.6 mL, 2.0 vol) at 0-5° C. The reaction mixture was stirred for 2 h at ambient temperature (25-30° C.). The reaction mixture was concentrated to remove the dichloromethane. The residue obtained was triturated with diethyl ether to afford Compound 9 as a white solid (0.23 g, 76% yield).

¹H NMR (400 MHz, DMSO) δ (ppm): 3.91 (s, 2H), 4.85 (s, 2H), 6.13 (m, 2H), 7.42 (dd, 4H), 7.64 (d, 2H), 8.17 (d, 2H).

LCMS: 467.2 [M+H].

HPLC: 98.2% purity at 8.55 minutes.

### Example 8: Preparation of 2-({[5-(4-chlorobenzamido)-2-[(4-chlorophenyl)methyl]-3-oxo-1,2,4-thiadiazolidin-4-yl]methoxy}carbonyl)-1-methylpyrrolidine (Compound 10)

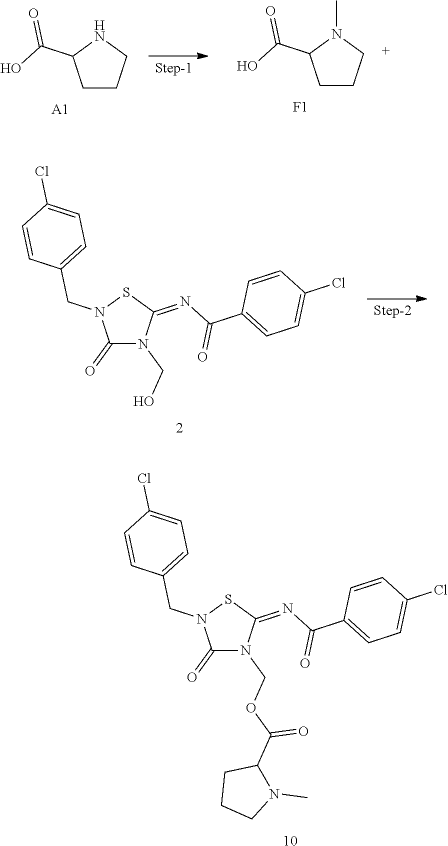

### Step-1: 1-methylpyrrolidine-2-carboxylic Acid (Compound F1)

To a solution of L-Proline (Compound A1; 2.0 g, 17.37 mmol) in methanol (20 mL), were added 37% formaldehyde (1.54 mL, 19.1 mmol) followed by Pd/C (0.5 g, 25% w/w) and the resulting mixture was hydrogenated in a Parr apparatus (1 kg H₂ pressure) for 16 h. After completion of the reaction, the reaction mixture was filtered through Celite® and concentrated under reduced pressure. The residue was triturated with ether to afford Compound F1 as a white solid (1.9 g, 84% yield)

### Step-2: 2-({[5-(4-chlorobenzamido)-2-[(4-chlorophenyl)methyl]-3-oxo-1,2,4-thiadiazolidin-4-yl]methoxy}carbonyl)-1-methylpyrrolidin-1-ium Trifluoroacetate (Compound 10)

To a stirring solution of Compound F1 (0.39 g, 3.0 mmol) in THF (10 mL, 20V) was added DMAP (0.074 g, 0.60 mmol) followed by DCC (0.62 g, 3.04 mmol) at 0-5° C. and stirred for 20 min. To the reaction mixture Compound 2 (0.5 g, 0.1.21 mmol) was added at 0-5° C. and the reaction mixture was stirred for 16 h at ambient temperature (25-30° C.). The reaction mixture was filtered, and the filtrate was concentrated and purified by reverse phase column chromatography followed by re-precipitation using acetone/diethyl ether to afford Compound 10 as a white solid (0.25 g, 39% yield).

¹H NMR (400 MHz, DMSO) δ (ppm): 1.83 (br, 1H), 2.03 (m, 2h), 2.40 (br, 1H), 2.87 (br, 1H), 3.13 (br, 1H), 3.57 (br, 1H), 4.85 (s, 2H), 6.13 (m, 2H), 7.41 (dd, 4H), 7.64 (d, 2H), 8.15 (d, 2H)

LCMS: 521.3 [M+H].

HPLC: 96.9% purity at 9.16 minutes.

### Example 9: Preparation of 1-{[5-(4-chlorobenzamido)-2-[(4-chlorophenyl)methyl]-3-oxo-1,2,4-thiadiazolidin-4-yl]methoxy}-1-oxopropan-2-aminium Trifluoroacetate (Compound 11)

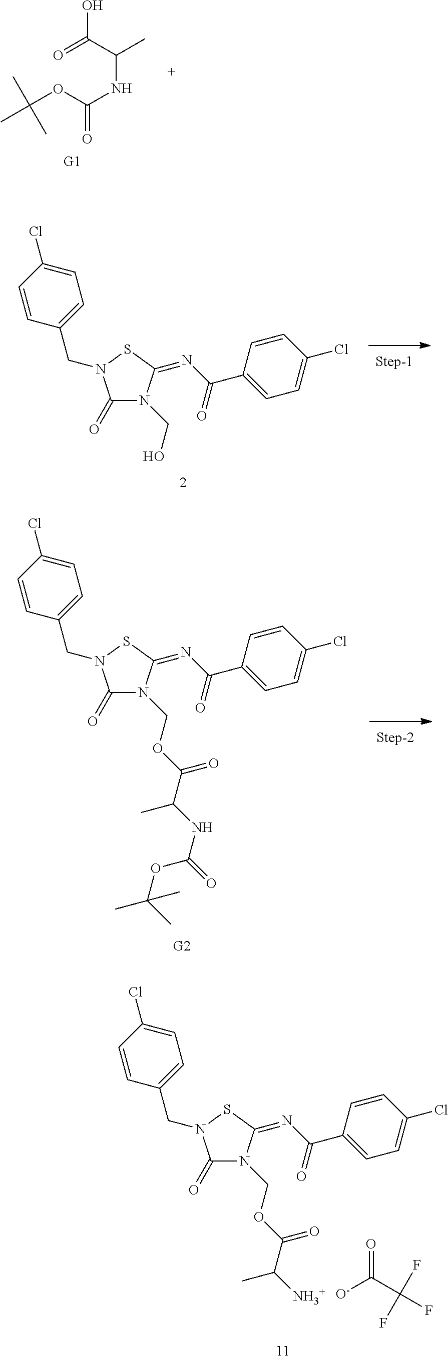

### Step-1: [5-(4-chlorobenzamido)-2-[(4-chlorophenyl)methyl]-3-oxo-1,2,4-thiadiazolidin-4-yl]methyl 2-{[(tert-butoxy)carbonyl]amino}propanoate (Compound G2)

To a stirring solution of N-Boc alanine (Compound G1) (0.57 g, 3.04 mmol) in THF (10 mL, 20V) was added DMAP (0.044 g, 0.36 mmol) followed by DCC (0.62 g, 3.04 mmol) at 0-5° C. and stirred for 20 min. To the reaction mixture Compound 2 (0.5 g, 1.21 mmol) was added at 0-5° C. and the reaction mixture was stirred for 16 h at ambient temperature (25-30° C.). The reaction mixture was filtered, and the filtrate was concentrated and purified by reverse phase column chromatography to afford Compound G2 as a white solid (0.31 g, 43% yield).

### Step-2: 1-{[5-(4-chlorobenzamido)-2-[(4-chlorophenyl)methyl]-3-oxo-1,2,4-thiadiazolidin-4-yl]methoxy}-1-oxopropan-2-aminium Trifluoroacetate (Compound 11)

To a stirring solution of Compound G2 (0.3 g, 0.51 mmol) in dichloromethane (3.0 mL) was added trifluoroacetic acid (0.9 mL, 3.0 vol) at 0-5° C. The reaction mixture was stirred for 2 h at ambient temperature (25-30° C.). The reaction mixture was concentrated to remove the dichloromethane. The residue obtained was triturated with diethyl ether to afford Compound 11 as a white solid (0.14 g, 45% yield).

¹H NMR (400 MHz, DMSO) δ (ppm): 1.33 (d, 3H), 4.19 (t, 1h), 4.85 (s, 2H), 6.12 (m, 2H), 7.41 (dd, 4H), 7.64 (m, 1H), 8.15 (d, 2H)

LCMS: 481.3 [M+H].

HPLC: 95.7% purity at 9.14 minutes.

### Example 10: Preparation of 1-{[5-(4-chlorobenzamido)-2-[(4-chlorophenyl)methyl]-3-oxo-1,2,4-thiadiazolidin-4-yl]methoxy}-3-methyl-1-oxopentan-2-aminium Trifluoroacetate (Compound 12)

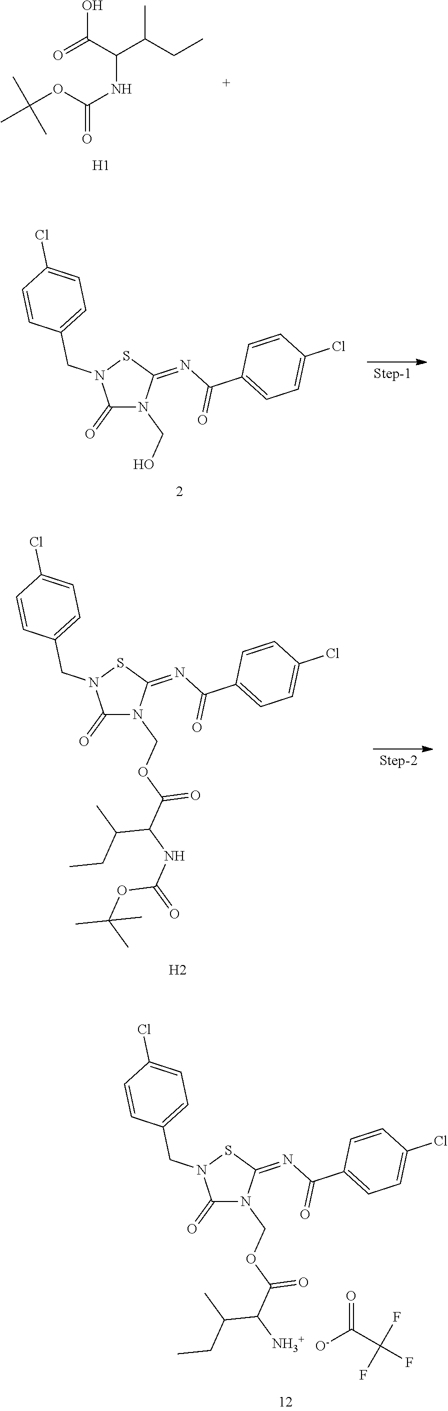

### Step-1: [5-(4-chlorobenzamido)-2-[(4-chlorophenyl)methyl]-3-oxo-1,2,4-thiadiazolidin-4-yl]methyl 2-{[(tert-butoxy)carbonyl]amino}-3-methylpentanoate (Compound H2)

To a stirring solution of N-Boc Isoleucine (Compound H1; 0.56 g, 3.04 mmol) in THF (10 mL, 20V) was added DMAP (0.044 g, 0.36 mmol) followed by DCC (0.62 g, 3.04 mmol) at 0-5° C. and stirred for 20 min. To the reaction mixture Compound 2 (0.5 g, 1.21 mmol) was added at 0-5° C. and the reaction mixture was stirred for 16 h at ambient temperature (25-30° C.). The reaction mixture was filtered, and the filtrate was concentrated and purified by reverse phase column chromatography to afford Compound H2 as a white solid (0.4 g, 52% yield).

### Step-2: 1-{[5-(4-chlorobenzamido)-2-[(4-chlorophenyl)methyl]-3-oxo-1,2,4-thiadiazolidin-4-yl]methoxy}-3-methyl-1-oxopentan-2-aminium Trifluoroacetate (Compound 12)

To a stirring solution of Compound H2 (0.4 g, 0.64 mmol) in dichloromethane (3.0 mL) was added trifluoroacetic acid (1.2 mL, 3.0 vol) at 0-5° C. The reaction mixture was stirred for 2 h at ambient temperature (25-30° C.). The reaction mixture was concentrated to remove the dichloromethane. The residue obtained was triturated with diethyl ether to afford Compound 12 as a white solid (0.22 g, 53% yield).

¹H NMR (400 MHz, DMSO) δ (ppm): 0.67 (t, 3H), 0.84 (d, 2H), 1.15 (m, 1H), 1.33 (m, 1H), 1.81 (br, 1H), 4.10 (s, 1h), 4.86 (s, 2H), 6.03 (d, 1H), 6.20 (d, 1H), 7.45 (dd, 4H), 7.64 (d, 1H), 8.17 (d, 2H)

LCMS: 523.3 [M+H].

HPLC: 98.0% purity at 10.38 minutes.

### Example 11: Preparation of [5-(4-chlorobenzamido)-2-[(4-chlorophenyl)methyl]-3-oxo-1,2,4-thiadiazolidin-4-yl]methyl 2-(dimethylamino)-3-phenylpropanoate (Compound 13)

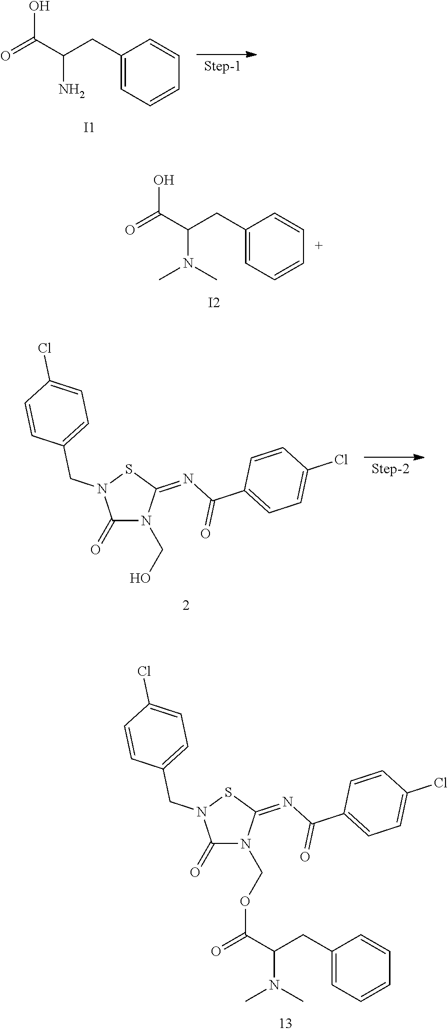

### Step-1: 2-(dimethylamino)-3-phenylpropanoic Acid (Compound I2)

To a solution of Phe-Ala-OH (Compound I1; 1.0 g, 6.05 mmol) in methanol (20 mL), were added 37% formaldehyde (1.96 mL, 24.21 mmol) followed by Pd/C (0.1 g, 10% w/w) and the resulting mixture was hydrogenated in a parr apparatus (5 kg H₂ pressure) for 16 h. After completion of the reaction, the reaction mixture was filtered through Celite® and concentrated under reduced pressure. The residue was triturated with ether to afford Compound 12 as a white solid (1.0 g, 86% yield).

### Step-2: [5-(4-chlorobenzamido)-2-[(4-chlorophenyl)methyl]-3-oxo-1,2,4-thiadiazolidin-4-yl]methyl 2-(dimethylamino)-3-phenylpropanoate (Compound 13)

To a stirring solution of Compound 12 (0.47 g, 2.43 mmol) in THF (10 mL, 20V) was added DMAP (0.044 g, 0.36 mmol) followed by DCC (0.62 g, 3.04 mmol) at 0-5° C. and stirred for 20 min. To the reaction mixture Compound 2 (0.5 g, 1.21 mmol) was added at 0-5° C. and the reaction mixture was stirred for 16 h at ambient temperature (25-30° C.). The reaction mixture was filtered, and the filtrate was concentrated and purified by reverse phase column chromatography to afford Compound 13 as a white solid (0.22 g, 30% yield).

¹H NMR (400 MHz, DMSO) δ (ppm): 2.49 (s, 6H), 2.92 (m, 2h), 3.51 (t, 1H), 4.89 (s, 2h), 5.97 (m, 2H), 7.07 (m, 5H), 7.40 (d, 2H), 7.45 (d, 2H), 7.64 (d, 2H), 8.15 (d, 2H).

LCMS: 586.0 [M+H].

HPLC: 97.5% purity at 11.54 minutes.

### Example 12: Preparation of 1-{[5-(4-chlorobenzamido)-2-[(4-chlorophenyl)methyl]-3-oxo-1,2,4-thiadiazolidin-4-yl]methoxy}-1-oxo-3-phenylpropan-2-aminium Trifluoroacetate (Compound 14)

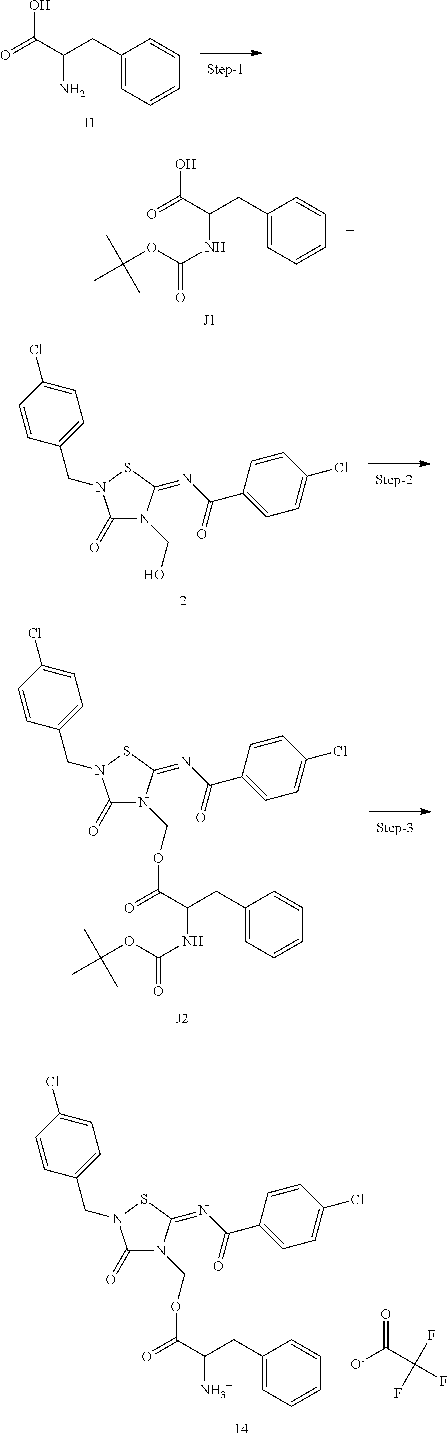

### Step-1: 2-{[(tert-butoxy)carbonyl]amino}-3-phenylpropanoic Acid (Compound J1)

To a solution of Phe-Ala-OH (Compound I1; 1.0 g, 6.05 mmol) in DCM (10 mL), was added triethylamine (1.70 mL, 12.17 mmol) at 0° C. followed by di-tert-butyl pyrocarbonate (2.18 mL, 9.08 mmol) and the resulting reaction mixture was stirred for 16 h at room temperature. The reaction mixture was carefully acidified with 5% citric acid solution to pH 4. The product was extracted with DCM, and the extract was dried, evaporated in a vacuum and purified by reverse phase column chromatography to afford Compound J1 as a colourless wax (1.3 g, 81% yield).

### Step-2: [5-(4-chlorobenzamido)-2-[(4-chlorophenyl)methyl]-3-oxo-1,2,4-thiadiazolidin-4-yl]methyl 2-{[(tert-butoxy)carbonyl]amino}-3-phenylpropanoate (Compound J2)

To a stirring solution of Compound J1 (0.64 g, 2.4 mmol) in THF (10 mL, 20V) was added DMAP (0.044 g, 0.36 mmol) followed by DCC (0.62 g, 3.04 mmol) at 0-5° C. and stirred for 20 min. To the reaction mixture Compound 2 (0.5 g, 1.21 mmol) was added at 0-5° C. and the reaction mixture was stirred for 16 h at ambient temperature (25-30° C.). The reaction mixture was filtered, and the filtrate was concentrated and purified by reverse phase column chromatography to afford Compound J2 as a white solid (0.3 g, 37% yield).

### Step-3: 1-{[5-(4-chlorobenzamido)-2-[(4-chlorophenyl)methyl]-3-oxo-1,2,4-thiadiazolidin-4-yl]methoxy}-1-oxo-3-phenylpropan-2-aminium Trifluoroacetate (Compound 14)

To a stirring solution of Compound J2 (0.3 g, 0.45 mmol) in dichloromethane (3.0 mL) was added trifluoroacetic acid (0.9 mL, 3.0 vol) at 0-5° C. The reaction mixture was stirred for 2 h at ambient temperature (25-30° C.). The reaction mixture was concentrated to remove the dichloromethane. The residue obtained was triturated with diethyl ether to afford Compound 14 as a white solid (0.2 g, 66% yield).

¹H NMR (400 MHz, DMSO) δ (ppm): 2.98 (m, 1H), 3.14 (m, 1H), 4.44 (m, 1H), 4.85 (s, 2h), 5.877 (s, 1H), 6.20 (d, 1H), 7.07 (m, 5H), 7.41 (d, 2H), 7.47 (d, 2H), 7.64 (d, 2H), 8.16 (d, 2H)

LCMS: 558.0 [M+H].

HPLC: 98.6% purity at 10.47 minutes.

### Example 13: Preparation of 1-{[5-(4-chlorobenzamido)-2-[(4-chlorophenyl)methyl]-3-oxo-1,2,4-thiadiazolidin-4-yl]methoxy}-4-methyl-1-oxopentan-2-aminium Trifluoroacetate (Compound 15)

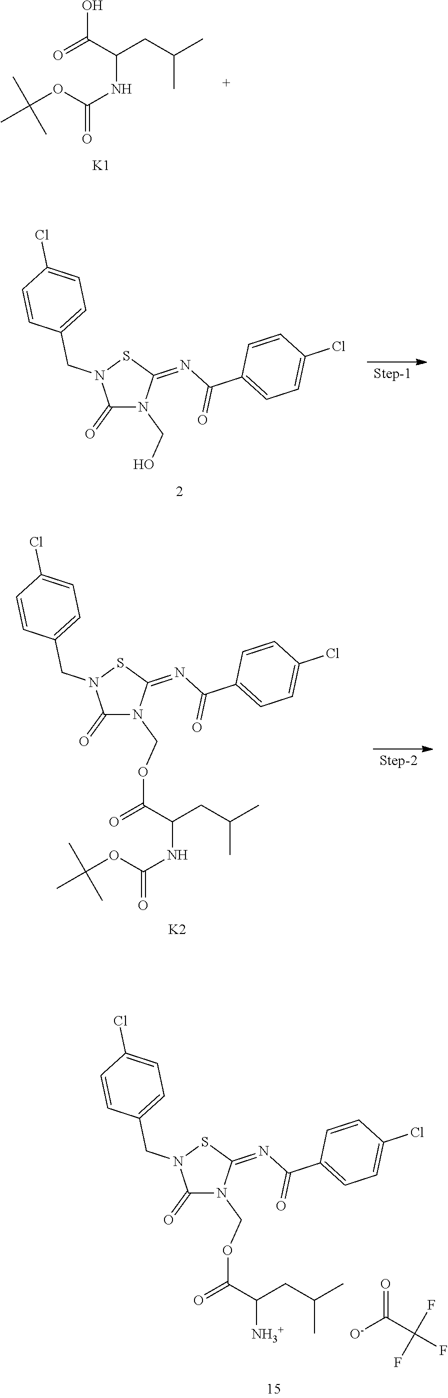

### Step-1: [5-(4-chlorobenzamido)-2-[(4-chlorophenyl)methyl]-3-oxo-1,2,4-thiadiazolidin-4-yl]methyl 2-{[(tert-butoxy)carbonyl]amino}-4-methyl Pentanoate (Compound K2)

To a stirring solution of 2-{[(tert-butoxy)carbonyl]amino}-4-methylpentanoic acid (Compound K1; 0.56 g, 2.43 mmol) in THF (10 mL, 20V) was added DMAP (0.044 g, 0.36 mmol) followed by DCC (0.62 g, 3.04 mmol) at 0-5° C. and stirred for 20 min. To the reaction mixture Compound 2 (0.5 g, 1.21 mmol) was added at 0-5° C. and the reaction mixture was stirred for 16 h at ambient temperature (25-30° C.). The reaction mixture was filtered, and the filtrate was concentrated and purified by reverse phase column chromatography to afford Compound K2 as a white solid (0.4 g, 75% yield).

### Step-2: 1-{[5-(4-chlorobenzamido)-2-[(4-chlorophenyl)methyl]-3-oxo-1,2,4-thiadiazolidin-4-yl]methoxy}-4-methyl-1-oxopentan-2-aminium Trifluoroacetate (Compound 15)

To a stirring solution of Compound K2 (0.35 g, 0.56 mmol) in dichloromethane (3.0 mL) was added trifluoroacetic acid (1.05 mL, 3.0 vol) at 0-5° C. The reaction mixture was stirred for 2 h at ambient temperature (25-30° C.). The reaction mixture was concentrated to remove the dichloromethane. The residue obtained was triturated with diethyl ether to afford Compound 15 as a white solid (0.27 g, 93% yield).

¹H NMR (400 MHz, DMSO) δ (ppm): 2.98 (m, 1H), 3.14 (m, 1H), 4.44 (m, 1H), 4.85 (s, 2h), 5.877 (s, 1H), 6.20 (d, 1H), 7.07 (m, 5H), 7.41 (d, 2H), 7.47 (d, 2H), 7.64 (d, 2H), 8.16 (d, 2H)

LCMS: 523.4 [M+H].

HPLC: 98.0% purity at 10.56 minutes.

### Example 14: Preparation of 1-{[5-(4-chlorobenzamido)-2-[(4-chlorophenyl)methyl]-3-oxo-1,2,4-thiadiazolidin-4-yl]methoxy}-3-hydroxy-1-oxopropan-2-aminium Trifluoroacetate (Compound 16)

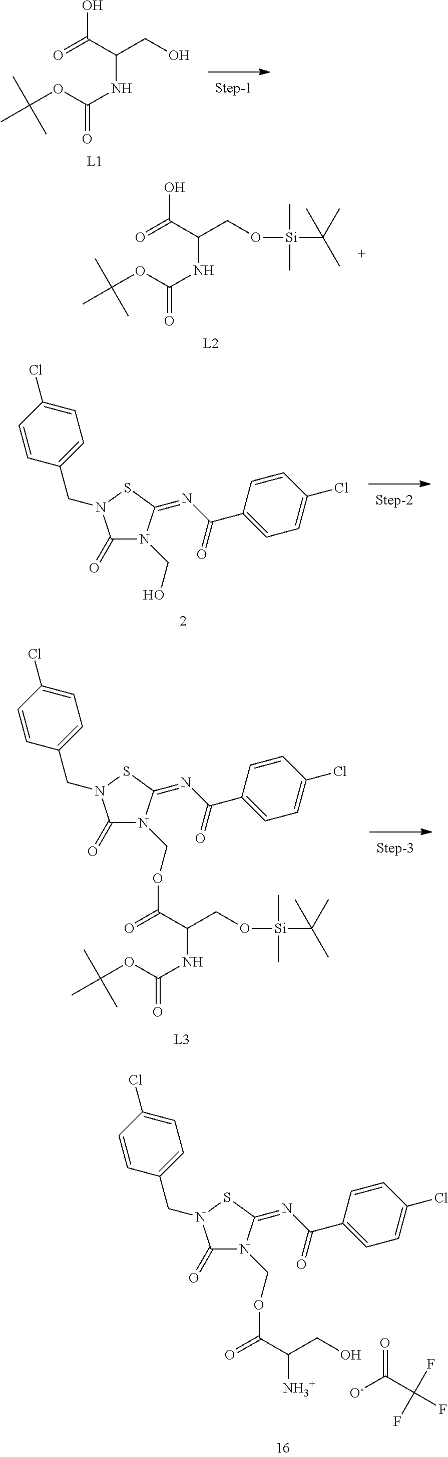

### Step-1: 2-{[(tert-butoxy)carbonyl]amino}-3-[(tert-butyldimethylsilyl) oxy]propanoic Acid (Compound L2)

To a solution of Boc-Ser-OH (Compound Li; 1.0 g, 4.87 mmol) in DCM (10 mL), was added imidazole (0.53 mg, 7.79 mmol) at 0° C. followed by TBDMS-Cl (1.10 g, 7.30 mmol) and the resulting reaction mixture was stirred for 16 h at room temperature. The product was extracted with DCM, and the extract was dried, evaporated in a vacuum and purified by reverse phase column chromatography to afford Compound L2 as a colourless wax (0.7 g, 46% yield).

### Step-2: [5-(4-chlorobenzamido)-2-[(4-chlorophenyl)methyl]-3-oxo-1,2,4-thiadiazolidin-4-yl]methyl 2-{[(tert-butoxy)carbonyl]amino}-3-[(tert-butyldimethyl silyl)oxy]propanoate (Compound L3)

To a stirring solution of Compound L2 (0.77 g, 2.43 mmol) in THF (10 mL, 20V) was added DMAP (0.044 g, 0.36 mmol) followed by DCC (0.62 g, 3.04 mmol) at 0-5° C. and stirred for 20 min. To the reaction mixture Compound 2 (0.5 g, 1.21 mmol) was added at 0-5° C. and the reaction mixture was stirred for 16 h at ambient temperature (25-30° C.). The reaction mixture was filtered, and the filtrate was concentrated and purified by reverse phase column chromatography to afford Compound L3 as a white solid (0.4 g, 46% yield).

### Step-3: 1-{[5-(4-chlorobenzamido)-2-[(4-chlorophenyl)methyl]-3-oxo-1,2,4-thiadiazolidin-4-yl]methoxy}-3-hydroxy-1-oxopropan-2-aminium Trifluoroacetate (Compound 16)

To a stirring solution of Compound L3 (0.4 g, 0.56 mmol) in dichloromethane (4.0 mL) was added trifluoroacetic acid (1.2 mL, 3.0 vol) at 0-5° C. The reaction mixture was stirred for 2 h at ambient temperature (25-30° C.). The reaction mixture was concentrated to remove the dichloromethane. The residue obtained was purified by reverse phase column chromatography to afford Compound 16 as a white solid (0.1 g, 29% yield).

¹H NMR (400 MHz, DMSO) δ (ppm): 3.80 (m, 2H), 4.27 (m, 1H), 4.85 (s, 2h), 5.60 (t, 1H), 6.12 (q, 21H), 7.07 (m, 5H), 7.44 (dd, 41H), 7.63 (d, 2H), 8.18 (d, 2H)

LCMS: 497.3 [M+H].

HPLC: 91.6% purity at 8.52 minutes.

### Example 15: Preparation of 3-carboxy-1-{[5-(4-chlorobenzamido)-2-[(4-chlorophenyl)methyl]-3-oxo-1,2,4-thiadiazolidin-4-yl]methoxy}-1-oxopropan-2-aminium Trifluoroacetate (Compound 17)

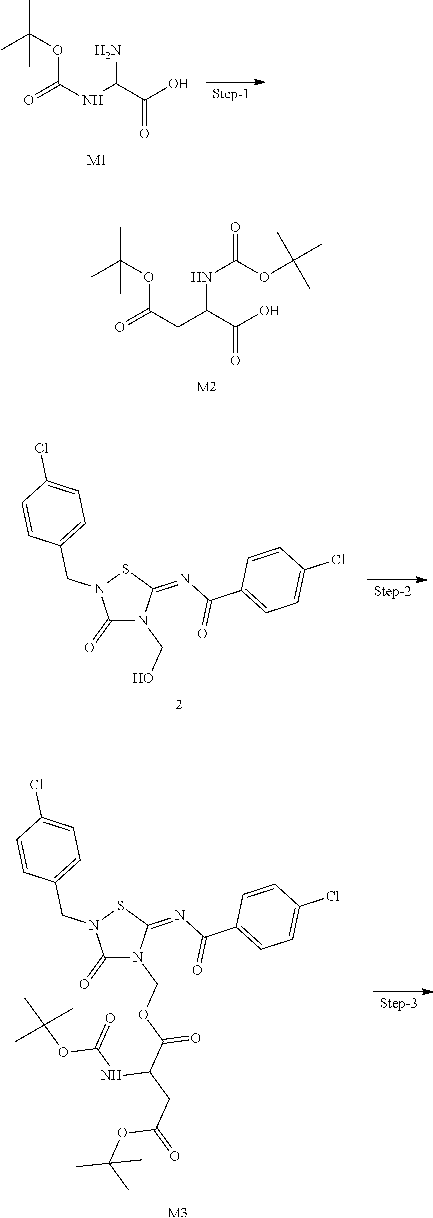

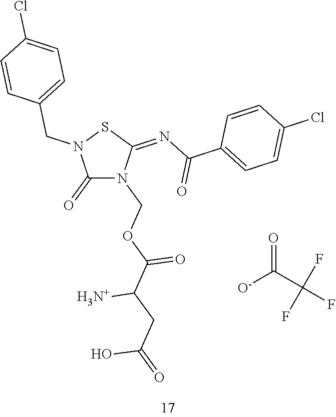 [SMILES: [NH3+]C(CC(=O)O)C(=O)OCn1c(=O)n(Cc2ccc(Cl)cc2)s/c1=N/C(=O)c1ccc(Cl)cc1 ; O=C([O-])C(F)(F)F]

### Step-1:4-(tert-butoxy)-2-{[(tert-butoxy) carbonyl]amino}-4-oxobutanoic Acid (Compound M2)

To a solution of L-Aspartic acid 4-tert-butyl ester (Compound M1; 2.0 g, 10.5 mmol) in dioxane:water (7:3, 20 mL), was added NaOH (0.84 g, 21.1 mmol) followed by di-tert-butyl pyrocarbonate (3.64 mL, 15.85 mmol) and the resulting reaction mixture was stirred for 16 h at room temperature. The reaction mixture was carefully acidified with 1.5N HCl solution to pH 3. The product was extracted with ethyl acetate, and the extract was dried and evaporated in a vacuum to afford Compound M2 as a white solid (2.5 g, 81% yield).

### Step-2: tert-butyl 1-[5-(4-chlorobenzamido)-2-[(4-chlorophenyl)methyl]-3-oxo-1,2,4-thiadiazolidin-4-yl]methyl 2-{[(tert-butoxy)carbonyl]amino}butanedioate (Compound M3)

To a stirring solution of Compound M2 (0.88 g, 3.04 mmol) in THF (18 mL, 20V) was added DMAP (0.044 g, 0.36 mmol) followed by DCC (0.62 g, 3.04 mmol) at 0-5° C. and stirred for 20 min. To the reaction mixture compound 2 (0.5 g, 1.21 mmol) was added at 0-5° C. and the reaction mixture was stirred for 16 h at ambient temperature (25-30° C.). The reaction mixture was filtered, and the filtrate was concentrated and purified by reverse phase column chromatography to afford Compound M3 as a white solid (0.45 g, 54% yield).

### Step-3: 3-carboxy-1-{[5-(4-chlorobenzamido)-2-[(4-chlorophenyl)methyl]-3-oxo-1,2,4-thiadiazolidin-4-yl]methoxy}-1-oxopropan-2-aminium Trifluoroacetate (Compound 17)

To a stirring solution of Compound M3 (0.45 g, 0.66 mmol) in dichloromethane (4.5 mL) was added trifluoroacetic acid (0.9 mL, 2.0 vol) at 0-5° C. The reaction mixture was stirred for 6 h at ambient temperature (25-30° C.). The reaction mixture was concentrated to remove the dichloromethane. The residue obtained was purified by reverse phase column chromatography to afford Compound 17 as a white solid (0.12 g, 28% yield).

¹H NMR (400 MHz, DMSO) δ (ppm): 2.85 (m, 2H), 4.41 (m, 1H), 4.84 (s, 2h), 6.12 (dd, 2H), 6.12 (q, 2H), 7.45 (dd, 4H), 7.63 (d, 2H), 8.18 (d, 2H)

LCMS: 525.3 [M+H].

HPLC: 94.2% purity at 8.29 minutes.

### Example 16—Preparation of [5-(4-chlorobenzamido)-2-[(4-chlorophenyl)methyl]-3-oxo-1,2,4-thiadiazolidin-4-yl]methyl 2-[(2S)-2-azaniumyl-3-phenylpropanamido] acetate Chloride (Compound 18)

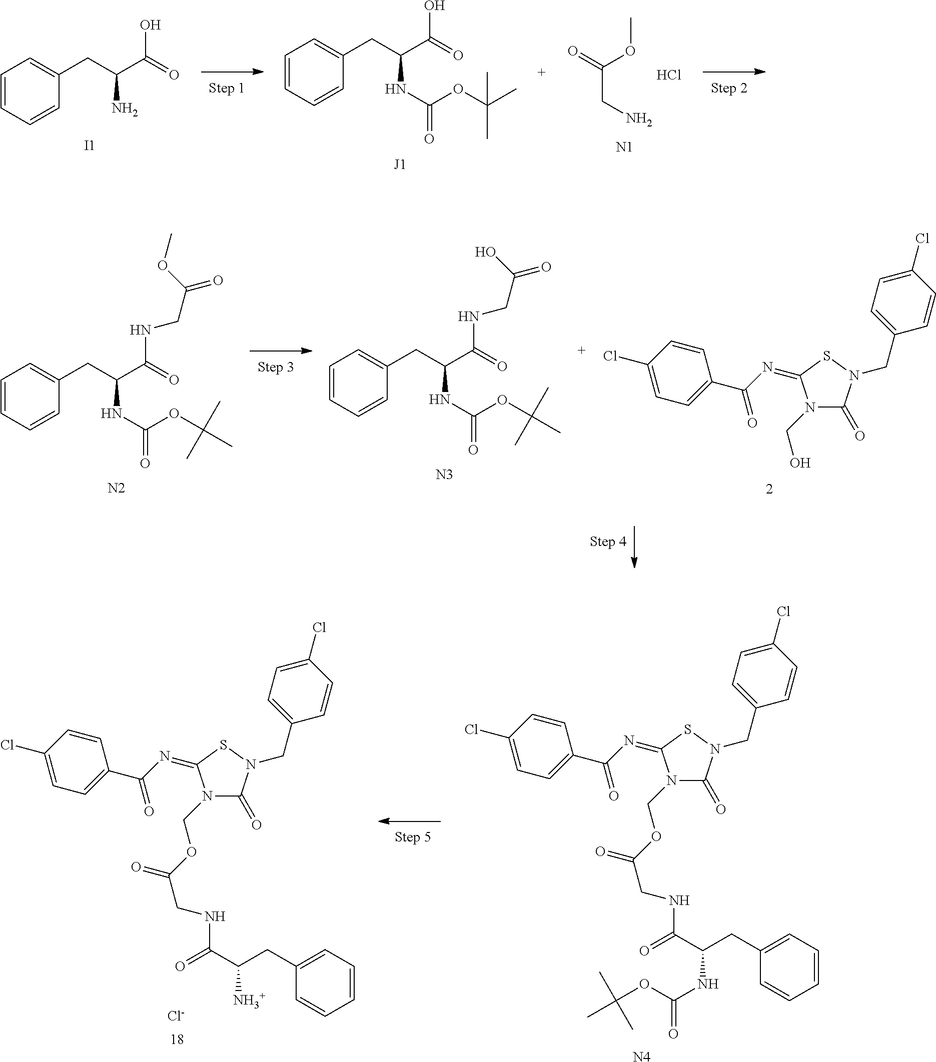

### Step-2: Preparation of Methyl 2-[(2S)-2-{[(tert-butoxy)carbonyl]amino}-3-phenylpropanamido]acetate (N2)

To a stirring solution of (2S)-2-{[(tert-butoxy)carbonyl]amino}-3-phenylpropanoic acid (J1, which was synthesized as described above; 2.6 g, 9.8 mmol) in DCM (52 mL, 20V) were added DCC (2.42 g, 11.76 mmol), DMAP (119 mg, 0.98 mmol) and triethyl amine (2.71 mL, 19.6 mmol) at 0-5° C. and stirred for 20 min. To the above reaction mixture methyl 2-aminoacetate hydrochloride (Compound Ni) (1.6 g, 12.74 mmol) was added at 0-5° C. and the reaction mixture was stirred for 16 h at ambient temperature (25-30° C.). The reaction mass was filtered to remove side product (urea), filtrate was concentrated and purified by silica gel (60-120 mesh) column chromatography to obtain Compound N2 as an off-white solid (2.73 g, 83% yield).

### Step-3: Preparation of 2-[(2S)-2-{[(tert-butoxy)carbonyl]amino}-3-phenylpropanamido]acetic Acid (Compound N3)

To a stirring solution of Compound N2 (3.0 g, 8.91 mmol) in methanol (30 mL, 10V) was added 2M NaOH solution (12 mL, 4V) at 0-5° C. and the reaction mixture was stirred for 16 h at ambient temperature (25-30° C.). The reaction mass was concentrated to remove volatiles, diluted with water (50 mL) and washed with MTBE (100 mL). The aqueous layer was carefully acidified with 1.5 N HCl solution to pH 4-5 and extracted with ethyl acetate (200 mL×2). The organic layers were combined, dried over Na2SO4 and concentrated under reduced pressure. The residue obtained was washed with hexanes (50 mL×2) to obtain Compound N3 as a white solid (1.6 g, 56% yield).

### Step-4: Preparation of [5-(4-chlorobenzamido)-2-[(4-chlorophenyl)methyl]-3-oxo-1,2,4-thiadiazolidin-4-yl]methyl 2-[(2S)-2-{[(tert-butoxy)carbonyl]amino}-3-phenylpropanamido]acetate (Compound N4)

To a stirring solution of compound N3 (1.6 g, 4.96 mmol) in THF (32 mL, 20V) was added DCC (1.53 g, 7.45 mmol) followed by DMAP (0.3 g, 2.48 mmol) at 0-5° C. and stirred for 20 min. To the resulting reaction mixture 4-chloro-N-{2-[(4-chlorophenyl)methyl]-4-(hydroxymethyl)-3-oxo-1,2,4-thiadiazolidin-5-ylidene}benzamide 6 (1.42 g, 3.47 mmol) was added at 0-5° C. and was allowed to stir for 16 h at ambient temperature (25-30° C.). The reaction mass was filtered; filtrate was concentrated and purified by reverse phase column chromatography to obtain Compound N4 as a white solid (354 mg, 10% yield).

### Step-5: Preparation of [5-(4-chlorobenzamido)-2-[(4-chlorophenyl)methyl]-3-oxo-1,2,4-thiadiazolidin-4-yl]methyl 2-[(2S)-2-azaniumyl-3-phenylpropanamido]acetate Chloride (Compound 18)

To a stirring solution of compound N4 (0.2 g, 0.279 mmol) in THF (1.0 mL, 5 vol), was added HCl in diethyl ether (4.0 mL, 20 vol) at 0-5° C. and the reaction mixture was stirred for 20 min at same temperature. The reaction mass was concentrated to remove volatiles and the residue obtained was triturated with diethyl ether to obtain Compound 18 as a white solid (150 mg, 83% yield).

¹H NMR (300 MHz, DMSO) δ (ppm): 2.94 (m, 1H), 3.15 (m, 1H), 4.05 (m, 3h), 4.84 (s, 2H), 6.07 (s, 2H), 7.27 (m, 5H), 7.42 (q, 4H), 7.62 (d, 2H), 8.17 (m, 2H), 8.23 (br, 2h, NH), 9.14 (t, 1H, NH).

LCMS: 614.31 [M+H].

HPLC: 97.35% purity at 11.82 minutes.

### Example 17—Preparation of [5-(4-chlorobenzamido)-2-[(4-chlorophenyl)methyl]-3-oxo-1,2,4-thiadiazolidin-4-yl]methyl (2S)-2-[(2S)-2-azaniumyl-3-methylbutanamido]-3-methylbutanoate Chloride (Compound 19)

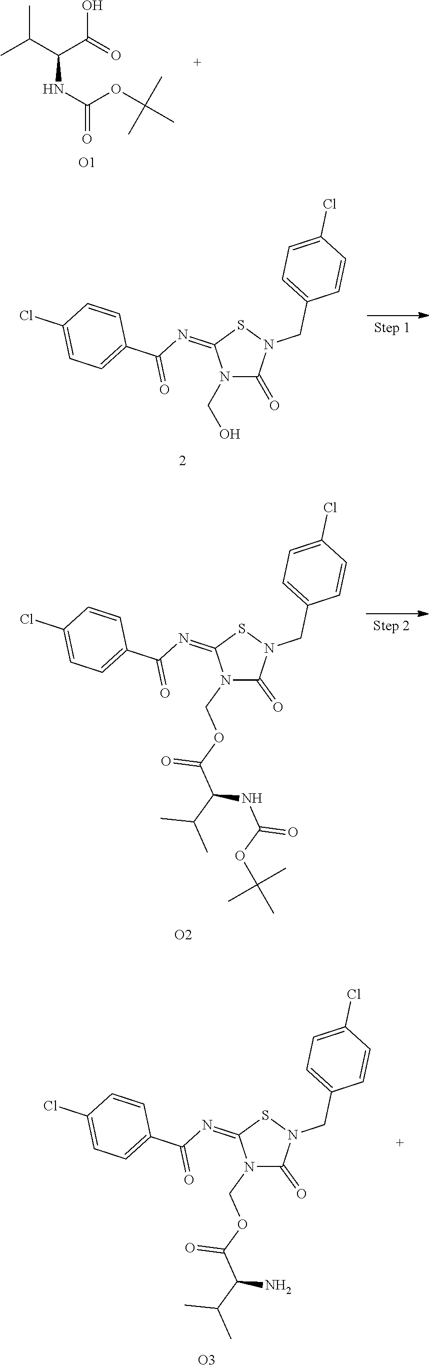

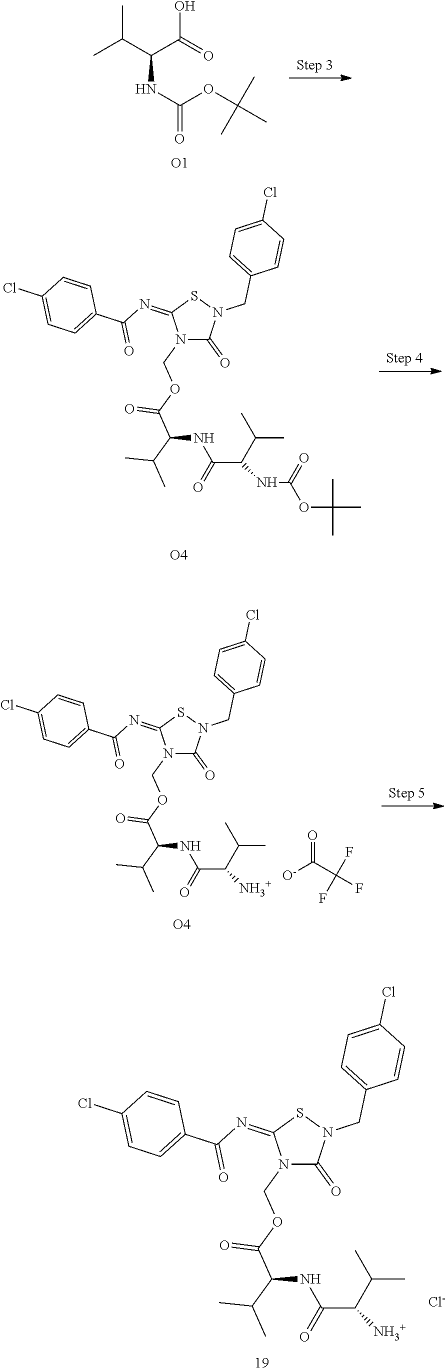

### Step-1: Preparation of [5-(4-chlorobenzamido)-2-[(4-chlorophenyl)methyl]-3-oxo-1,2,4-thiadiazolidin-4-yl]methyl (2S)-2-{[(tert-butoxy)carbonyl]amino}-3-methylbutanoate (Compound O2)

To a stirring solution of (2S)-2-{[(tert-butoxy)carbonyl]amino}-3-methylbutanoic acid (01; 3.0 g, 13.8 mmol) in THF (60 mL, 20V) was added DMAP (0.84 g, 6.91 mmol) followed by DCC (4.27 g, 20.73 mmol) at 0-5° C. and stirred for 20 min. To the above reaction mixture Compound 2 (2.83 g, 6.91 mmol) was added at 0-5° C. and the resulting reaction mixture was stirred for 16 h at ambient temperature (25-30° C.). The reaction mass was diluted with dichloromethane (200 mL), washed with water (2×100 mL) and concentrated under reduced pressure to obtain Compound O2 as white solid (6.0 g, 77%).

NOTE: The crude compound 02 was carried to next step without any purification.

### Step-2: Preparation of (2S)-1-{[5-(4-chlorobenzamido)-2-[(4-chlorophenyl)methyl]-3-oxo-1,2,4-thiadiazolidin-4-yl]methoxy}-3-methyl-1-oxobutan-2-aminium Trifluoroacetate (Compound O3)

To a stirring solution of [5-(4-chlorobenzamido)-2-[(4-chlorophenyl)methyl]-3-oxo-1,2,4-thiadiazolidin-4-yl]methyl (2S)-2-{[(tert-butoxy)carbonyl]amino}-3-methylbutanoate (Compound O2; 6.0 g, 9.85 mmol) in dichloromethane (25 mL) was added trifluoroacetic acid (24 mL, 4.0 vol) at 0-5° C. and stirred for 4 h at ambient temperature (25-30° C.). The reaction mixture was concentrated to remove volatiles and the residue obtained was triturated with ether and filtered under vacuum to obtain Compound O3 as a white solid (1.5 g, 24% yield).

### Step-3: Preparation of [5-(4-chlorobenzamido)-2-[(4-chlorophenyl)methyl]-3-oxo-1,2,4-thiadiazolidin-4-yl]methyl (2S)-2-(2-{[(tert-butoxy)carbonyl]amino}-3-methylbutanamido)-3-methylbutanoate (Compound O4)

To a stirring solution of (2S)-2-{[(tert-butoxy)carbonyl]amino}-3-methylbutanoic acid (Compound O1; 1.30 g, 6.02 mmol) in THF (30 mL, 20V) was added DMAP (0.088 g, 0.72 mmol) followed by DCC (1.39 g, 6.75 mmol) at 0-5° C. and stirred for 20 min. To the above reaction mixture (2S)-1-{[5-(4-chlorobenzamido)-2-[(4-chlorophenyl)methyl]-3-oxo-1,2,4-thiadiazolidin-4-yl]methoxy}-3-methyl-1-oxobutan-2-aminium trifluoroacetate (Compound O3; 1.5 g, 2.41 mmol) was added at 0-5° C. and was stirred for 16 h at ambient temperature (25-30° C.). The reaction mixture was filtered; filtrate was concentrated and purified by reverse phase column chromatography to obtain Compound O4 as white solid (0.65 g, 37% yield).

### Step-4: Preparation of [5-(4-chlorobenzamido)-2-[(4-chlorophenyl)methyl]-3-oxo-1,2,4-thiadiazolidin-4-yl]methyl (2S)-2-[(2S)-2-azaniumyl-3-methylbutanamido]-3-methylbutanoate Trifluoroacetate (Compound O5)

To a stirring solution of [5-(4-chlorobenzamido)-2-[(4-chlorophenyl)methyl]-3-oxo-1,2,4-thiadiazolidin-4-yl]methyl (2S)-2-(2-{[(tert-butoxy)carbonyl]amino}-3-methylbutanamido)-3-methylbutanoate (Compound O4; 0.66 g, 0.93 mmol) in dichloromethane (25 mL) was added trifluoroacetic acid (1.98 mL, 3.0 vol) at 0-5° C. and the reaction mixture was stirred for 4 h at ambient temperature (25-30° C.). The reaction mixture was concentrated to remove volatiles and the residue obtained was triturated with ether and filtered under vacuum to obtain Compound O5 as a white solid (0.45 g, 67% yield).

### Step-5: Preparation of [5-(4-chlorobenzamido)-2-[(4-chlorophenyl)methyl]-3-oxo-1,2,4-thiadiazolidin-4-yl]methyl (2S)-2-[(2S)-2-azaniumyl-3-methylbutanamido]-3-methylbutanoate Chloride (Compound 19)

To a stirring solution of [5-(4-chlorobenzamido)-2-[(4-chlorophenyl)methyl]-3-oxo-1,2,4-thiadiazolidin-4-yl]methyl (2S)-2-[(2S)-2-azaniumyl-3-methylbutanamido]-3-methylbutanoate trifluoroacetate (Compound O5; 0.45 g, 0.62 mmol) in THF (2.25 mL, 5.0 vol) was added HCl in diethyl ether (9.0 mL, 20 vol) at 0-5° C. and the reaction mixture was stirred for 20 min at same temperature. The reaction mass was concentrated to remove volatiles and the residue obtained was triturated with diethyl ether to obtain Compound 19 as a white solid (240 mg, 60% yield).

¹H NMR (300 MHz, DMSO) δ (ppm): 0.8-0.9 (bm, 12H, —CH₃), 2.07 (m, 2H), 3.7 (dd, 1H), 4.26 (m, 1H), 4.84 (s, 2h), 5.97 (dd, 1H), 6.11 (q, 1H), 7.43 (dd, 4H), 7.63 (d, 2H), 8.15 (m, 2K), 8.19 (m, 3H, NH₃+), 8.6 (m, 1H, NH)

LCMS: 608.53 [M+H].

HPLC: 79.5% purity at 11.125 minutes+17.3% purity at 11.97 minutes (rotamers)

### Example 18—Preparation of [5-(4-chlorobenzamido)-2-[(4-chlorophenyl)methyl]-3-oxo-1,2,4-thiadiazolidin-4-yl]methyl (2R)-2-[(2R)-2-azaniumyl-3-methylbutanamido]-3-methylbutanoate Chloride (Compound 20)

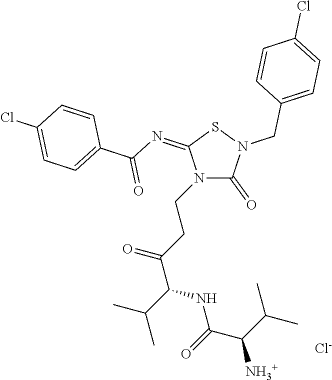 [SMILES: CC(C)[C@@H]([NH3+])C(=O)N[C@@H](C(=O)CCn1c(=O)n(Cc2ccc(Cl)cc2)s/c1=N/C(=O)c1ccc(Cl)cc1)C(C)C ; [Cl-]]

The methods used were analogous to those described for the preparation of Compound 19 in Example 17, starting from (2R)-2-{[(tert-butoxy)carbonyl]amino}-3-methylbutanoic acid, to give Compound 20 as a white solid (300 mg, 88% yield).

¹H NMR (300 MHz, DMSO) δ (ppm): 0.8-0.9 (bm, 12H, —CH₃), 2.07 (m, 2H), 3.69 (dd, 1H), 4.26 (m, 1H), 4.84 (s, 2h), 5.97 (dd, 1H), 6.11 (q, 1H), 7.43 (dd, 4H), 7.63 (d, 2H), 8.15 (m, 2K), 8.19 (m, 3H, NH₃⁺), 8.6 (m, 1H, NH)

LCMS: 608.53 [M+H].

HPLC: 75.93% purity at 13.43 minutes+22.45% purity at 14.5 minutes (rotamers)

### Example 19—Preparation of [5-(4-chlorobenzamido)-2-[(4-chlorophenyl)methyl]-3-oxo-1,2,4-thiadiazolidin-4-yl]methyl 2-[(2S)-2,6-diazaniumylhexanamido]acetate Dichloride (Compound 21)

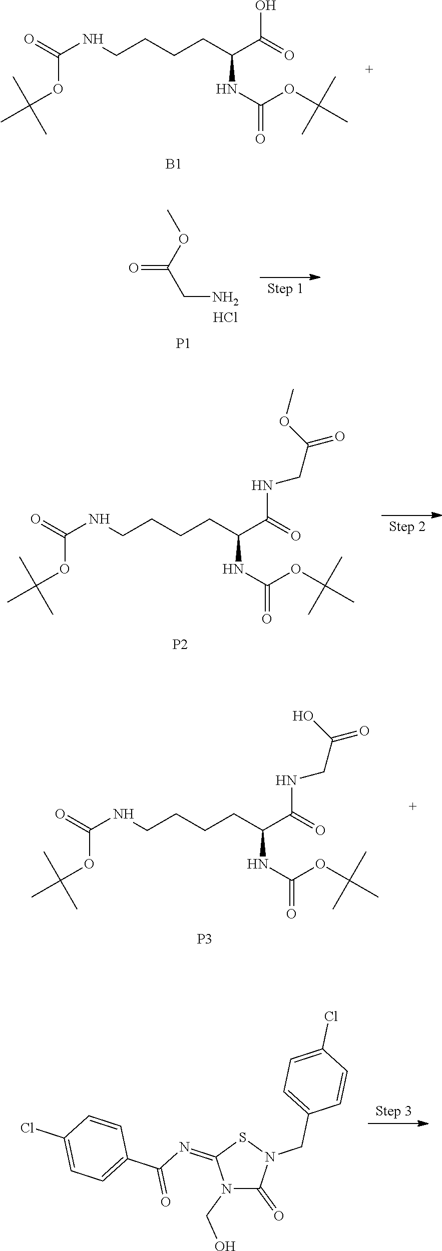

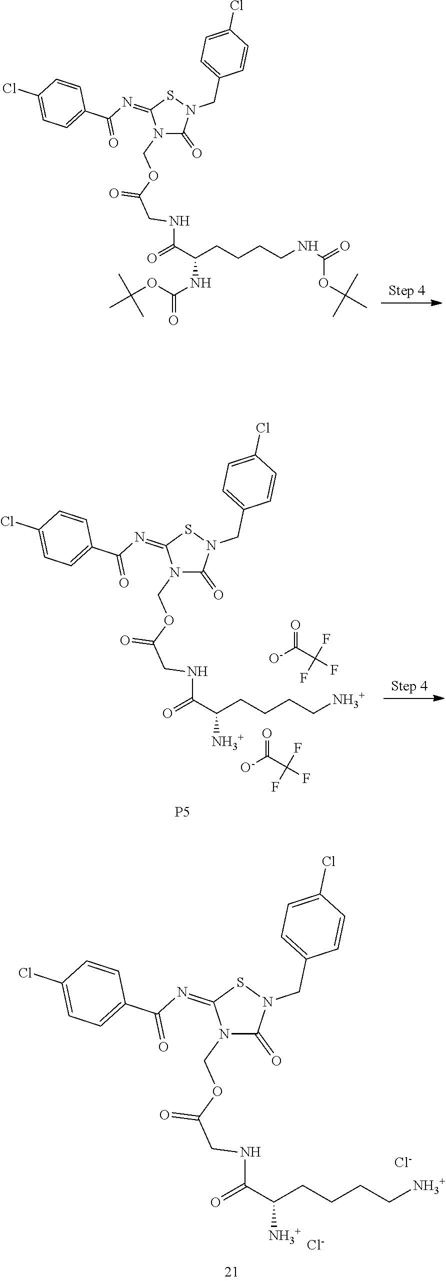

### Step-1: Preparation of Methyl 2-[(2S)-2,6-bis({[(tert-butoxy)carbonyl]amino}) hexanamido]acetate (Compound P2)

To a stirring solution of (2S)-2,6-bis({[(tert-butoxy)carbonyl]amino})hexanoic acid (Compound B1; 2.6 g, 7.51 mmol) in DCM (52 mL, 20V) were added DCC (3.86 g, 18.78 mmol), DMAP (275 mg, 2.25 mmol) and triethyl amine (2.07 mL, 15.02 mmol) at 0-5° C. and stirred for 20 min. To the above reaction mixture methyl 2-aminoacetate hydrochloride P1 (939 mg, 7.51 mmol) was added at 0-5° C. and was further stirred for 16 h at ambient temperature (25-30° C.). The reaction mass was filtered to remove side product (urea) and filtrate was concentrated under reduced pressure. The residue obtained was purified by silica gel column chromatography to afford Compound P2 as a colourless liquid (1.81 g, 58% yield).

### Step-2: Preparation of 2-[(2S)-2,6-bis({[(tert-butoxy)carbonyl]amino}) hexanamido]acetic Acid (Compound P3)

To a stirring solution of methyl 2-[(2S)-2,6-bis({[(tert-butoxy)carbonyl]amino})hexanamido]acetate (Compound P2; 3.0 g, 7.19 mmol) in methanol (30 mL, 10V) was added 2M NaOH solution (12 mL, 4V) at 0-5° C. The resulting reaction mixture was allowed to stir at ambient temperature (25-30° C.) for 16 h. The reaction mass was concentrated to remove methanol, diluted with water (50 mL) and washed with MTBE (50 mL×2). The aqueous layer was carefully acidified with 1.5N HCl solution to pH 4-5 and extracted with ethyl acetate (100 mL×2). The organic layers were combined, dried over Na2SO4 and concentrated under reduced pressure. The residue was washed with hexanes to obtain Compound P3 as a colourless liquid (2.75 g, 95% yield).

### Step-3: Preparation of [5-(4-chlorobenzamido)-2-[(4-chlorophenyl)methyl]-3-oxo-1,2,4-thiadiazolidin-4-yl]methyl 2-[(2S)-2,6-bis({[(tert-butoxy)carbonyl]amino}) hexanamido]acetate (Compound P4)

To a stirring solution of (2-[(2S)-2,6-bis({[(tert-butoxy)carbonyl]amino})hexanamido]acetic acid (Compound P3; 1.75 g, 4.34 mmol) in THF (35 mL, 20V) was added DMAP (0.26 g, 2.17 mmol) followed by EDC·HCl (1.24 g, 6.51 mmol) at 0-5° C. and stirred for 20 min. To the above reaction Compound 2 (0.89 g, 2.17 mmol) was added at 0-5° C. and stirred for 16 h at ambient temperature (25-30° C.). The reaction mass was diluted with dichloromethane (200 mL), washed with water (2×100 mL) and concentrated under reduced pressure. The residue obtained upon evaporation of the solvents was purified by reverse phase column chromatography to afford Compound P4 as a white solid (0.3 g, 12% yield).

### Step-4: Preparation of [5-(4-chlorobenzamido)-2-[(4-chlorophenyl)methyl]-3-oxo-1,2,4-thiadiazolidin-4-yl]methyl 2-[(2S)-2,6-diazaniumylhexanamido]acetate di-trifluoroacetate (Compound P5)

To a stirring solution of [5-(4-chlorobenzamido)-2-[(4-chlorophenyl)methyl]-3-oxo-1,2,4-thiadiazolidin-4-yl]methyl 2-[(2S)-2,6-bis({[(tert-butoxy)carbonyl]amino}) hexanamido]acetate (Compound P4; 0.3 g, 0.37 mmol) in dichloromethane (4.5 mL, 15V) was added trifluoroacetic acid (1.2 mL, 4 vol) at 0-5° C. and was stirred for 4 h at ambient temperature (25-30° C.). The reaction mixture was concentrated to remove volatiles and the residue obtained was washed with ether, filtered and dried under vacuum to obtain Compound P5 as a white solid (0.25 g, 96% yield).

### Step-5: Preparation of [5-(4-chlorobenzamido)-2-[(4-chlorophenyl)methyl]-3-oxo-1,2,4-thiadiazolidin-4-yl]methyl 2-[(2S)-2,6-diazaniumylhexanamido]acetate di-chloride (Compound 21)

To a stirring solution of [5-(4-chlorobenzamido)-2-[(4-chlorophenyl)methyl]-3-oxo-1,2,4-thiadiazolidin-4-yl]methyl 2-[(2S)-2,6-diazaniumylhexanamido]acetate di-trifluoroacetate (Compound P5; 0.11 g, 0.15 mmol) in THF (0.5 mL, 5.0 vol) was added HCl in diethyl ether (2.2 mL, 20 vol) at 0-5° C. and the reaction mixture was stirred for 20 min at same temperature. The reaction mass was concentrated to remove volatiles and the residue obtained was triturated with diethyl ether to obtain Compound 21 as a white solid (90 mg, 90% yield).

¹H NMR (300 MHz, DMSO) δ (ppm): 1.44 (br, 2H), 1.60 (br, 2H), 1.75 (br, 2H), 2.78 (br, 2H), 3.87 (br, 1H), 4.04 (m, 2h), 4.86 (s, 2H), 6.05 (s, 2H), 7.45 (m, 4H), 7.65 (d, 2H), 8.00 (br, 3H, NH₃⁺), 8.18 (d, 2H). 8.35 (br, 3H, NH₃⁺), 9.14 (br, 1H, NH)

LCMS: 595.49 [M+H].

HPLC: 94.09% purity at 8.68 minutes.

### Example 20—Preparation of (1S)-2-carboxy-1-[(2-{[5-(4-chlorobenzamido)-2-[(4-chlorophenyl)methyl]-3-oxo-1,2,4-thiadiazolidin-4-yl]methoxy}-2-oxoethyl)carbamoyl]ethan-1-aminium Chloride (Compound 22)

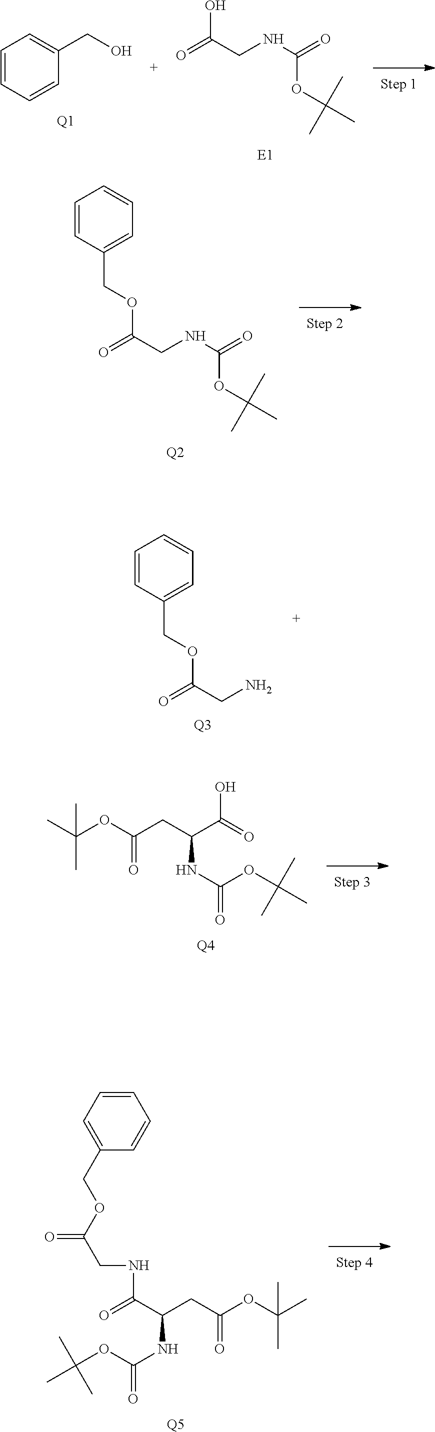

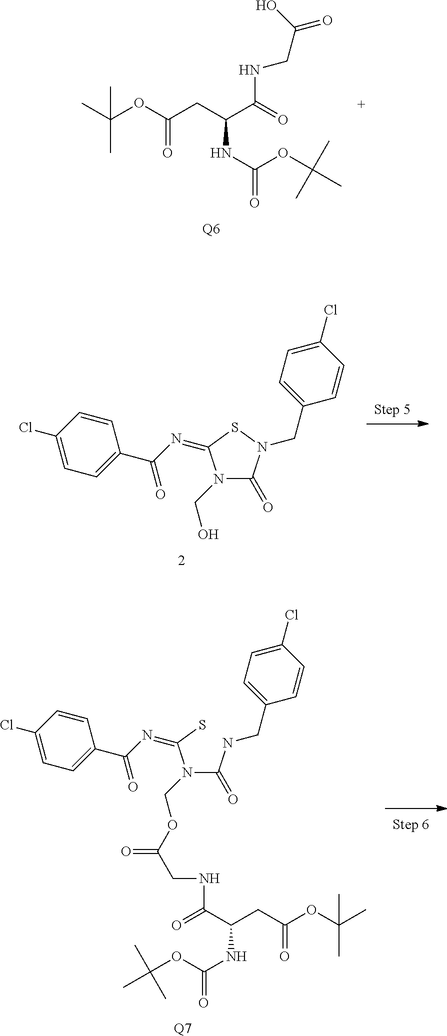

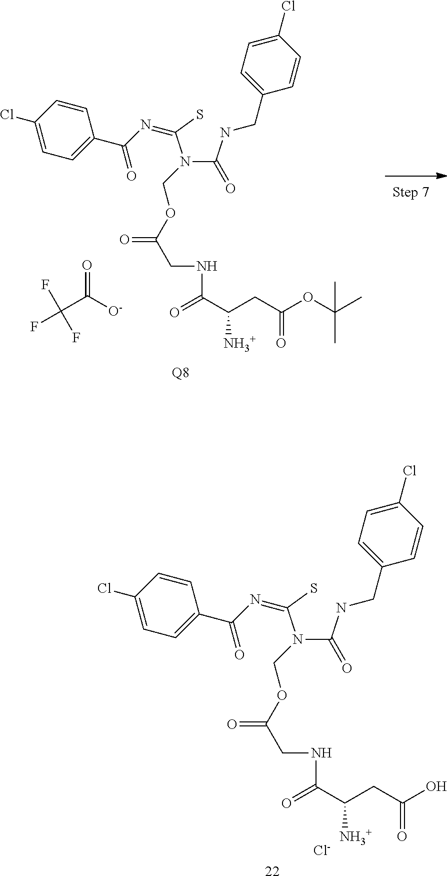 [SMILES: CC(C)(C)OC(=O)C[C@H]([NH3+])C(=O)NCC(=O)OCN(C(=O)NCc1ccc(Cl)cc1)/C(S)=N\C(=O)c1ccc(Cl)cc1 ; O=C([O-])C(F)(F)F ; "Q8" ; [NH3+][C@@H](CC(=O)O)C(=O)NCC(=O)OCN(C(=O)NCc1ccc(Cl)cc1)/C(S)=N\C(=O)c1ccc(Cl)cc1 ; [Cl-] ; "22"]

### Step-1: Preparation of Benzyl 2-{[(tert-butoxy)carbonyl]amino}acetate (Compound Q2)

To a stirring solution of 2-{[(tert-butoxy)carbonyl]amino}acetic acid (Compound E1; 2.0 g, 11.42 mmol) in DCM (40 mL, 20V) was added DMAP (130 mg, 1.14 mmol) followed by EDC·HCl (2.65 g, 17.12 mmol) at 0-5° C. and the resulting mixture was stirred for 5 minutes. To the above reaction mixture phenylmethanol (Q1; 1.35 g, 12.55 mmol) was added at 0-5° C. and was allowed to stir for 16 h at ambient temperature (25-30° C.).

The reaction mass was diluted with dichloromethane (200 mL, washed with water (2×100 mL) and concentrated under reduced pressure. The residue obtained upon evaporation of the solvents was purified by silica gel flash column chromatography to obtain Compound Q2 as a white solid (2 g, 66% yield).

### Step-2: Preparation of Benzyl 2-aminoacetate (Compound Q3)

To a stirring solution of benzyl 2-{[(tert-butoxy)carbonyl]amino}acetate (Compound Q2; 2 g, 7.54 mmol) in dichloromethane (30 mL, 15V) was added trifluoroacetic acid (4 mL, 2 V) at 0-5° C. The reaction mixture was allowed to stir for 3 h at ambient temperature (25-30° C.). The reaction mass was concentrated to remove the volatiles and the residue obtained was triturated with diethyl ether to afford Compound Q3 as a white solid (1.8 g, 85% yield).

### Step-3: Preparation of Tert-Butyl (3S)-3-{[2-(benzyloxy)-2-oxoethyl]carbamoyl}-3-{[(tert-butoxy)carbonyl]amino}propanoate (Compound Q5)

To a stirring solution of (2S)-4-(tert-butoxy)-2-{[(tert-butoxy)carbonyl]amino}-4-oxobutanoic acid (Compound Q4; 4.37 g, 15.1 mmol) in DCM (20 mL, 20V) was added DMAP (140 mg, 1.21 mmol) followed by EDC·HCl (3.23 g, 16.95 mmol) at 0-5° C. and stirred for 5 min. To the above reaction mixture benzyl 2-aminoacetate (Compound Q3; 1.0 g, 6.05 mmol) and DIPEA (2.96 mL, 18.16 mmol) were added at 0-5° C. and the reaction mixture was stirred for 16 h at ambient temperature (25-30° C.). The reaction mass was diluted with dichloromethane (400 mL), washed with water (2×200 mL) and concentrated under reduced pressure. The residue obtained upon evaporation of the solvents was purified by silica gel flash column chromatography to obtain Compound Q5 as a pale yellow wax (1 g, 64% yield).

### Step-4: Preparation of 2-[(2S)-4-(tert-butoxy)-2-{[(tert-butoxy)carbonyl]amino}-4-oxobutanamido]acetic Acid (Compound Q6)

To a solution of tert-butyl (3S)-3-{[2-(benzyloxy)-2-oxoethyl]carbamoyl}-3-{[(tert-butoxy)carbonyl] amino}propanoate (Compound Q5; 1.0 g, 2.29 mmol) in ethyl acetate (30 mL, 30V), was added Pd/C (0.1 g, 10% w/w) and the resulting mass was hydrogenated in a parr apparatus (5 kg H2 pressure) for 3 h. The reaction mass was filtered through celite pad and concentrated under reduced pressure. The residue obtained was triturated with diethyl ether to afford Compound Q6 as a white solid (0.67 g, 84% yield).

### Step-5: Preparation of tert-butyl (3S)-3-{[(tert-butoxy)carbonyl]amino}-3-[(2-{[5-(4-chlorobenzamido)-2-[(4-chlorophenyl)methyl]-3-oxo-1,2,4-thiadiazolidin-4-yl]methoxy}-2-oxoethyl)carbamoyl]propanoate (Compound Q7)

To a solution of 2-[(2S)-4-(tert-butoxy)-2-{[(tert-butoxy)carbonyl]amino}-4-oxobutanamido]acetic acid (Compound Q6; 0.64 g, 1.87 mmol) in THF (7 mL, 20V) was added DMAP (0.052 g, 0.42 mmol) followed by DCC (0.43 g, 2.13 mmol) at 0-5° C. and stirred for 20 min. To the above reaction mixture Compound 2 (0.35 g, 0.85 mmol) was added at 0-5° C. and the reaction mixture was stirred for 16 h at ambient temperature (25-30° C.). The reaction mixture was filtered; filtrate was concentrated and purified by reverse phase column chromatography to obtain Compound Q7 as a white solid (0.5 g, 79% yield).

### Step-6: Preparation of (1S)-2-carboxy-1-[(2-{[5-(4-chlorobenzamido)-2-[(4-chlorophenyl)methyl]-3-oxo-1,2,4-thiadiazolidin-4-yl]methoxy}-2-oxoethyl)carbamoyl]ethan-1-aminium Trifluoroacetate (Compound Q8)

To a stirring solution of tert-butyl (3S)-3-{[(tert-butoxy)carbonyl]amino}-3-[(2-{[5-(4-chlorobenzamido)-2-[(4-chlorophenyl)methyl]-3-oxo-1,2,4-thiadiazolidin-4-yl]methoxy}-2-oxoethyl)carbamoyl]propanoate (Compound Q7; 0.5 g, 0.67 mmol) in dichloromethane (7.5 mL) was added trifluoroacetic acid (2 mL, 4.0 vol) at 0-5° C. and was stirred for 4 h at ambient temperature (25-30° C.). The reaction mass was concentrated to remove the volatiles and the residue obtained was triturated with diethyl ether to afford Compound Q8 as a white solid (0.25 g, 53% yield).

### Step-7: Preparation of (1S)-2-carboxy-1-[(2-{[5-(4-chlorobenzamido)-2-[(4-chlorophenyl)methyl]-3-oxo-1,2,4-thiadiazolidin-4-yl]methoxy}-2-oxoethyl)carbamoyl]ethan-1-aminium Chloride (Compound 22)

To a stirring solution (1S)-2-carboxy-1-[(2-{[5-(4-chlorobenzamido)-2-[(4-chlorophenyl)methyl]-3-oxo-1,2,4-thiadiazolidin-4-yl]methoxy}-2-oxoethyl)carbamoyl]ethan-1-aminium trifluoroacetate (Compound Q8; 0.25 g, 0.35 mmol) in THF (1.25 mL, 5.0 vol) was added HCl in diethyl ether (5.0 mL, 20 vol) at 0-5° C. and the reaction mixture was stirred for 20 min at same temperature. The reaction mass was concentrated to remove volatiles and the residue obtained was triturated with diethyl ether to obtain Compound 22 as a white solid (200 mg, 90% yield).

¹H NMR (400 MHz, DMSO) δ (ppm): 2.85 (m, 2H), 4.07 (m, 3H), 4.83 (s, 2h), 6.04 (s, 2H), 7.44 (m, 4H), 7.65 (d, 2H), 7.63 (d, 2H), 8.14 (d, 2H), 8.25 (br, 3H, NH₃⁺), 9.01 (s, 1H), 12-14 (br, 1H, —COOH).

LCMS: 582.3 M+H].

HPLC: 95.26% purity at 11.43 minutes.

### Example 21—Preparation of [azaniumyl({[(4S)-4-azaniumyl-4-[(2-{[5-(4-chlorobenzamido)-2-[(4-chlorophenyl)methyl]-3-oxo-1,2,4-thiadiazolidin-4-yl]methoxy}-2-oxoethyl)carbamoyl]butyl]amino})methylidene]azanium Trichloride (Compound 23)

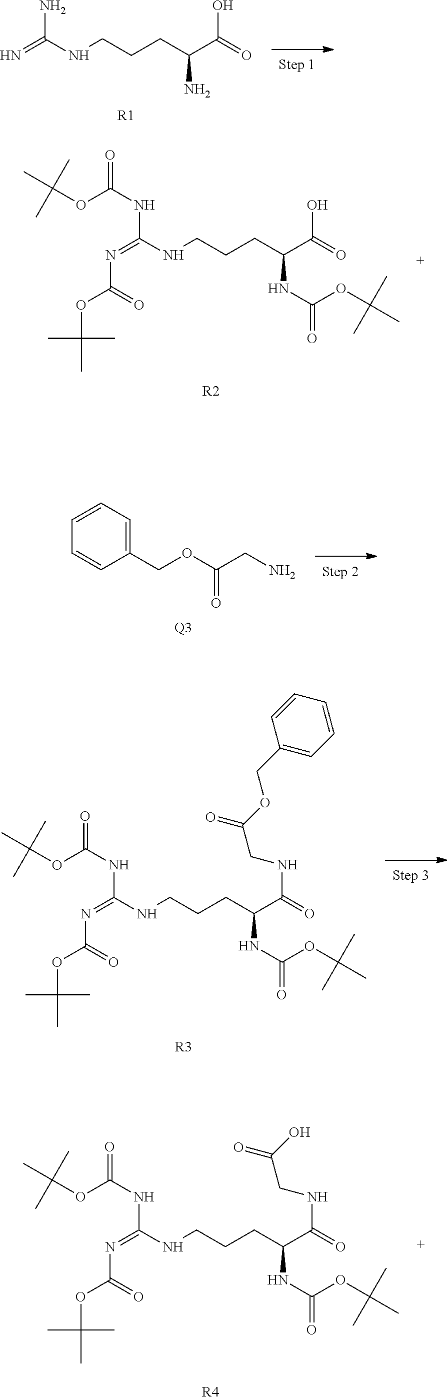

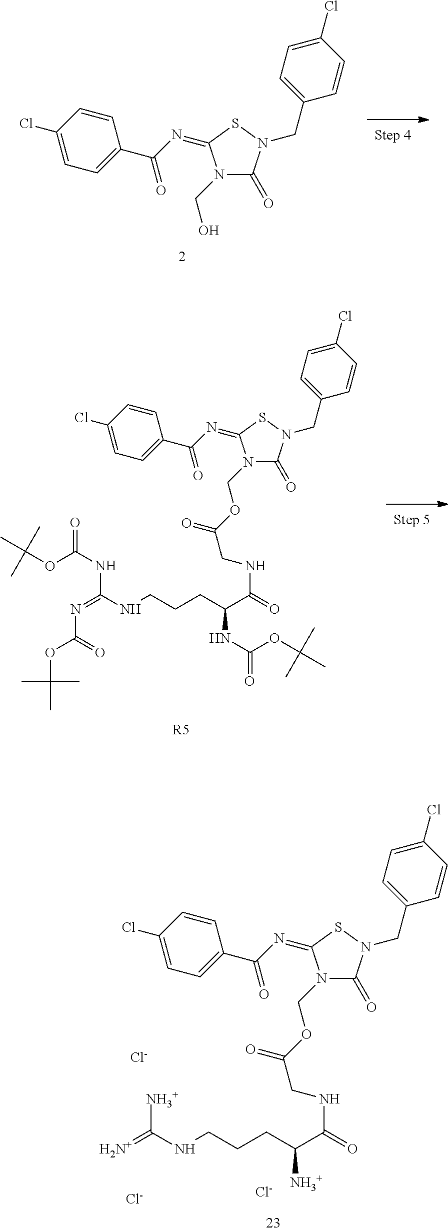

### Step-1: Preparation of (2S)-2-{[(tert-butoxy)carbonyl]amino}-5-{[(1E)-{[(tert-butoxy)carbonyl] amino}({[(tert-butoxy)carbonyl]imino})methyl]amino}pentanoic Acid (Compound R2)

To a solution of (2S)-2-amino-5-carbamimidamidopentanoic acid (Compound R1; 8.8 g, 50.51 mmol) in t-Butanol (158 mL, 18V) and water (158 mL, 18V), were added NaOH (7.07 g, 176.80 mmol) and Boc2O (46.42 mL, 202.06 mmol) at 0° C. The reaction mass was allowed to stir for 48 h at ambient temperature (25-30° C.). Evaporated the volatiles from the reaction mass and acidified carefully with saturated citric acid solution to pH 3-4. The product was extracted with ethyl acetate, and the extract was dried over Na2SO4, filtered, evaporated in a vacuum and purified by reverse phase column chromatography to obtain Compound R2 as a white solid (3.7 g, 15% yield).

### Step-2: Preparation of Benzyl 2-[(2S)-2-{[(tert-butoxy)carbonyl]amino}-5-{[(1E)-{[(tert-butoxy) carbonyl]amino}({[(tert-butoxy)carbonyl]imino})methyl]amino}pentanamido] acetate (Compound R3)

To a stirring solution of (2S)-2-{[(tert-butoxy)carbonyl]amino}-5-{[(1E)-{[(tert-butoxy)carbonyl] amino}({[(tert-butoxy)carbonyl]imino})methyl]amino}pentanoic acid (Compound R2; 1.33 g, 2.15 mmol) in THF (8 mL, 20V) was added DMAP (0.052 g, 0.43 mmol) followed by EDC·HCl (0.54 g, 2.86 mmol) at 0-5° C. and stirred for 20 min. To the above reaction mixture, benzyl aminoacetate (Compound Q3; 0.4 g, 1.43 mmol) in TEA (0.6 mL, 4.30 mmol) was added at 0-5° C. and the reaction mixture was stirred for 16 h at ambient temperature (25-30° C.). The reaction mass was diluted with water (100 mL), product was extracted with dichloromethane (200 mL), and the extract was dried and evaporated in a vacuum. The residue was purified by silica gel column chromatography to obtain Compound R3 as a white solid (0.6 g, 67% yield).

### Step-3: Preparation of 2-[(2S)-2-{[(tert-butoxy)carbonyl]amino}-5-{[(1E)-{[(tert-butoxy)carbonyl] amino}({[(tert-butoxy)carbonyl]imino})methyl]amino} pentanamido]acetic Acid (Compound R4)

To a solution of benzyl 2-[(2S)-2-{[(tert-butoxy)carbonyl]amino}-5-{[(1E)-{[(tert-butoxy)carbonyl]amino}({[(tert-butoxy)carbonyl]imino})methyl]amino} pentanamido]acetate (Compound R3; 0.6 g, 0.96 mmol) in ethyl acetate (6 mL, 10V), was added Pd/C (0.06 g, 10% w/w) and hydrogenated in a parr apparatus (5 kg H2 pressure) for 3 h. The reaction mass was filtered through celite bed and the filtrate was concentrated under reduced pressure. The residue was washed with ether to afford Compound R4 as an off white solid (0.43 g, 84% yield).

### Step-4: Preparation of [5-(4-chlorobenzamido)-2-[(4-chlorophenyl)methyl]-3-oxo-1,2,4-thiadiazolidin-4-yl]methyl 2-[(2S)-2-{[(tert-butoxy)carbonyl]amino}-5-{[(1E)-{[(tert-butoxy)carbonyl]amino}({[(tert-butoxy)carbonyl]imino})methyl]amino} pentanamido] acetate (Compound R5)

To a stirring solution of 2-[(2S)-2-{[(tert-butoxy)carbonyl]amino}-5-{[(1E)-{[(tert-butoxy)carbonyl]amino}({[(tert-butoxy)carbonyl]imino})methyl]amino}pentanamido]acetic acid (Compound R4; 0.41 g, 0.77 mmol) in THF (3.2 mL, 20V) was added DMAP (0.014 g, 0.11 mmol) followed by DCC (0.18 g, 0.89 mmol) at 0-5° C. and stirred for 20 min. To the above reaction mixture Compound 2 (0.16 g, 0.38 mmol) was added at 0-5° C. and further stirred for 16 h at ambient temperature (25-30° C.). The reaction mixture was filtered; filtrate was concentrated and purified by reverse phase column chromatography to obtain Compound R5 as a white solid (0.25 g, 69% yield).

### Step-5: Preparation of [azaniumyl({[(4S)-4-azaniumyl-4-[(2-{[5-(4-chlorobenzamido)-2-[(4-chlorophenyl)methyl]-3-oxo-1,2,4-thiadiazolidin-4-yl]methoxy}-2-oxoethyl)carbamoyl]butyl]amino})methylidene]azanium Trichloride (Compound 23)

To a stirring solution of [5-(4-chlorobenzamido)-2-[(4-chlorophenyl)methyl]-3-oxo-1,2,4-thiadiazolidin-4-yl]methyl 2-[(2S)-2-{[(tert-butoxy)carbonyl]amino}-5-{[(1E)-{[(tert-butoxy)carbonyl]amino}({[(tert-butoxy)carbonyl]imino})methyl]amino}pentanamido]acetate (Compound R5; 0.2 g, 0.21 mmol) in THF (1.0 mL, 5.0 vol) was added HCl in diethyl ether (4.0 mL, 20 vol) at 0-5° C. and the reaction mixture was stirred for 4 h at same temperature. The reaction mass was concentrated to remove volatiles and the residue obtained was triturated with diethyl ether to obtain Compound 23 as a white solid (90 mg, 60% yield).

¹H NMR (400 MHz, DMSO) δ (ppm): 1.45 (br, 3H), 1.78 (br, 4H), 3.14 (m, 2H), 3.88 (m, 1H), 4.05 (m, 2H), 4.84 (s, 2H), 6.04 (s, 2H), 7.45 (q, 5H), 7.65 (dd, 2H), 7.83 (t, 1H), 8.18 (d, 2H), 8.35 (br, 4H), 9.20 (t, 1H)

LCMS: 623.3 [M+H].

HPLC: 92.07% purity at 8.52 minutes.

### Example 22—Stability of Compounds

5 μM solutions of Compound 1 and Compound 4 (i.e. the citric acid salt of Compound 3) in HCl buffer (pH: 1.20) were prepared with 5 mM DMSO stock solutions. The solutions were incubated at 37° C. for 120 minutes, with shaking at 400 rpm using a thermomixer.

After 0, 15, 30, 60 and 120 minute intervals, aliquots of sufficient volume of the compound solutions were taken, diluted and analysed to determine the stability of Compounds 1 and 3 in acidic solutions.

### Results

The results for the stability experiments are tabulated in Table 3 below.

The results show that Compound 3 possesses greater stability than Compound 1 in acidic solutions (i.e. pH=1.2).

TABLE 3
Comparative Stability Data in HCl Buffer (pH: 1.20)

| | % remaining compared to 0.0 min | | | | |
|---|---|---|---|---|---|
| Test Compound | 0.0 min | 15 min | 30 min | 60 min | 120 min |
| Compound 1 | 100.00 | 71.32 | 63.05 | 58.77 | 43.04 |
| Compound 3 | 100.00 | 96.92 | 83.74 | 86.54 | 88.31 |

### Example 23—Activation of AMPK

### Cultivation and Compound Treatment of INS-1E Insulinoma Cells

INS-1E cells were cultivated as described in Steneberg et al., JCI Insight. 2018; 3(12):e99114. https://doi.org/10.1172/jci.insight.99114, using 5% instead of 10% fetal bovine serum when plated for treatment.

Compound 3 was dissolved at 10 mM in DMSO and frozen at −20° C.

The INS-1E cells were treated in serum free medium with increasing doses of Compound 3 for 4 hours in accordance with the methods described in Steneberg et al., JCI Insight. 2018; 3(12):e99114. https://doi.org/10.1172/jci.insight.99114.

### Western Blot Analysis

Western blot analysis of the INS-1E cells was performed as described in Steneberg et. al., JCI Insight. 2018; 3(12):e99114. https://doi.org/10.1172/jci.insight.99114. The cell lysates were passed through a 30-gauge needle, ˜8-times and centrifuged for 10 min at +4° C. at 14000 rpm. Quantified values for AMPKα, and phosphorylated-T172 AMPKα, were normalized toward the quantified values for β-Actin.

### Results

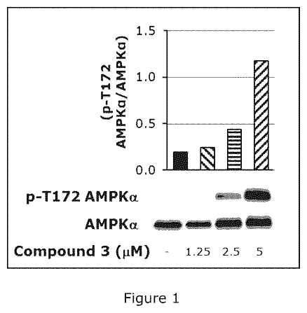

The results for Western blot analysis are tabulated in Table 4 below and are shown graphically in FIG. 1.

The results show that Compound 3 increases phosphorylated-T172 AMPK in a dose-dependent manner in cultured INS-1E cells. Compound 3 is therefore an agonist of AMPK.

TABLE 4
The ratio between p-T172 AMPK and non-phosphorylated AMPK

| Compound | Time | Control | 1.25 μM | 2.5 μM | 5 μM |
|---|---|---|---|---|---|
| 3 | 4 h | 1.00 | 1.24 | 2.24 | 5.99 |

### Example 24—Single Dose Oral Pharmacokinetic Study of Compounds 1 and 3 in Sprague Dawley Rats

### Formulation of Compound 1

10.0 mL of 2% w/v methyl cellulose solution in phosphate buffer (pH 7.5) was added to a 100 mL conical flask along with 1 g of 2 mm glass beads. The solution was vigorously stirred on a magnetic stirrer. 100 mg of Compound 1 was slowly added to the solution, and the solution was vigorously stirred for approximately 1 h. The pH of the formulation was measured to be 7.49. Each formulation was freshly prepared prior to administration to the animals. The final concentration of Compound 1 in of the formulation was 10 mg/mL. The formulation was administered at 5 mL/kg body weight.

### Formulation of Compound 3

6 mL of 2% w/v methyl cellulose solution in phosphate buffer (pH 7.5) was added to a 25 mL of conical flask together with 1 g of 2 mm glass beads. The solution was vigorously stirred on a magnetic stirrer. 78 mg of Compound 3 was slowly added to the solution, and the solution was vigorously stirred for approximately 1 h. The pH of the formulation was measured to be 7.41. Each formulation was freshly prepared prior to administration to the animals. The final concentration of Compound 3 in the formulation was 13 mg/mL, which is equivalent to 10 mg/mL of Compound 1. The formulation was administered at 5 mL/kg body weight.

### Dose Selection and Justification for Selection

The doses of 50 mg/kg and 65 mg/kg b.w. for Compound 1 and Compound 3, respectively, were selected for comparison of equimolar doses.

### Dose Administration

Adult healthy male Sprague Dawley rats aged 8 to 10 weeks were used for experimentation after a minimum three days of acclimatization. Fed state animals were administered the formulation of Compound 1 or Compound 3 orally via gavage at a dose of 50 mg/kg or 65 mg/kg body weight, respectively.

### Blood Sampling

Under mild isoflurane anesthesia, blood specimens were collected by retro-orbital puncture method using capillary tubes into pre-labeled tubes containing anticoagulant (K₂EDTA: 2 mg/mL blood) during the next 72 hours post-dosing, as detailed in Table 6. For blood sampling at multiple time points, the right and left eyes were used alternatively. Collected blood specimens were centrifuged at 6000 rpm, 4° C. for 10 minutes and the plasma samples were separated and stored at −80° C. until analysis.

### Results

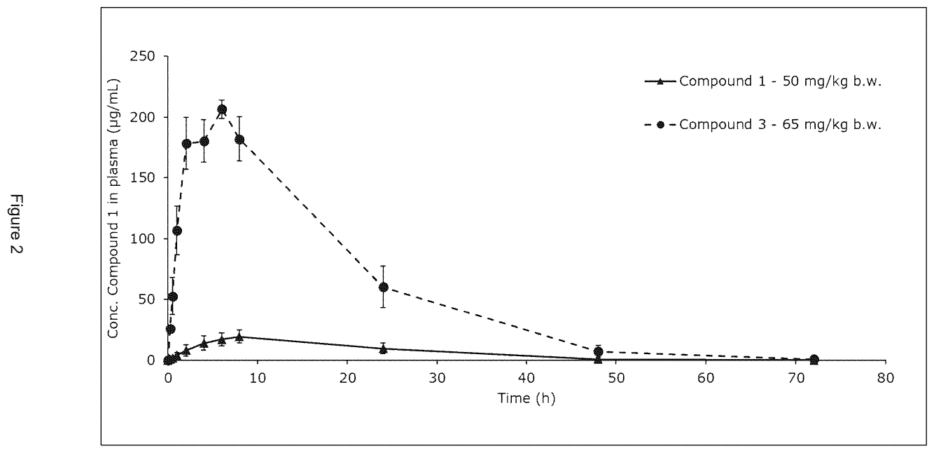

The results for the single dose oral pharmacokinetic study in rats are tabulated in Tables 5 and 6 below and are shown graphically in FIG. 2. In Table 5, the Cₘₐₓ, Tₘₐₓ, AUCₗₐₛₜ, AUC_{inf}, AUCₑₓₜᵣₐₚ, T_{1/2} and MRTₗₐₛₜ values are for 4-chloro-N-[2-[(4-chlorophenyl)methyl]-3-oxo-1,2,4-thiadiazol-5-yl]benzamide (Compound 1), which was detected following the administration of both Compounds 1 and 3.

The results show that there is a surprising ten-fold increase in the systemic exposure of 4-chloro-N-[2-[(4-chlorophenyl)methyl]-3-oxo-1,2,4-thiadiazol-5-yl]benzamide when Compound 3 is administered compared to Compound 1 (Cmax was 19.60 μg of Compound 1 per mL following administration of Compound 1 compared to 207.00 μg of Compound 1 per mL when administering Compound 3). Thus, the systemic exposure of 4-chloro-N-[2-[(4-chlorophenyl)methyl]-3-oxo-1,2,4-thiadiazol-5-yl]benzamide is increased by administration of that compound in the form of a compound of the invention.

TABLE 5
Mean Plasma Pharmacokinetic Parameters for 4-Chloro-N-[2-[(4-
chlorophenyl)methyl]-3-oxo-1,2,4-thiadiazol-5-yl]benzamide Following
Administration of Compounds 1 and 3

| | Compound No. | |
|---|---|---|
| | 1 | 3 |
| Dose (mg/kg b.w.) | 50 | 65 |
| | | (equivalent to 50 of Compound 1) |
| Cₘₐₓ (μg/mL) | 19.60 ± 5.54 | 207.00 ± 7.00 |
| Tₘₐₓ (h) | 7.33 ± 1.16 | 4.67 ± 2.31 |
| AUCₗₐₛₜ (h * μg/mL) | 449.40 ± 157.75 | 4161.45 ± 599.30 |
| AUC_{inf} (h * μg/mL) | 458.31 ± 154.77 | 4173.93 ± 593.40 |
| AUCₑₓₜᵣₐₚ (%) | 2.33 ± 1.74 | 0.32 ± 0.22 |
| T_{1/2} (h) | 8.42 ± 1.47 | 7.30 ± 1.01 |
| MRTₗₐₛₜ (h) | 14.78 ± 0.68 | 12.99 ± 2.12 |

TABLE 6
Plasma Concentration of Compound 1 Following
Administration of Compounds 1 and 3

| Plasma concentration of Compound 1 and 3 (μg/mL) | | | | |
|---|---|---|---|---|
| | Compound 1 | | Compound 3 | |
| Time (h) | Mean | SD | Mean | SD |
| 0.00 | 0.00 | 0.00 | 0.00 | 0.00 |
| 0.25 | 0.62 | 0.62 | 25.67 | 2.73 |
| 0.50 | 1.98 | 0.59 | 52.80 | 15.35 |
| 1.00 | 4.24 | 1.84 | 106.77 | 19.99 |
| 2.00 | 7.99 | 4.49 | 178.33 | 21.50 |
| 4.00 | 14.01 | 5.73 | 180.33 | 17.47 |
| 6.00 | 17.00 | 5.30 | 206.67 | 7.51 |
| 8.00 | 19.33 | 5.33 | 182.00 | 18.25 |
| 24.00 | 9.44 | 4.43 | 60.27 | 17.13 |
| 48.00 | 0.72 | 0.17 | 7.19 | 4.91 |
| 72.00 | 0.00 | 0.00 | 0.62 | 0.34 |

Lower limit of quantification of Compound 1 0.5 μg/mL

### Example 25 Single Dose Oral Pharmacokinetic Study of Compounds 5, 6, 7, 9, 10, 13, 14, 16 in Sprague Dawley Rats

### Formulation of the Compounds

Formulations were prepared in accordance with the method described in Example 19.

### Dose Selection and Justification for Selection

Doses equivalent to 50 mg/kg of Compound 1 were selected for comparison of equimolar doses.

### Dose Administration and Blood Sampling

The administration and blood sampling were performed as described in Example 19.

### Results

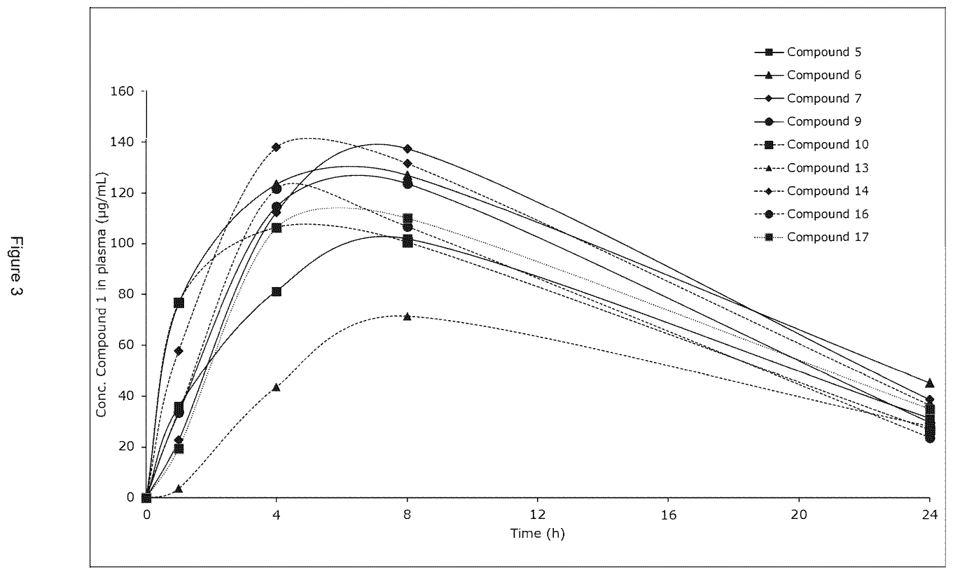

The results for the single dose oral pharmacokinetic study in rats are tabulated in Tables 7 and below and are shown graphically in FIG. 3. In Table 7, the Cₘₐₓ, Tₘₐₓ, AUCₗₐₛₜ, AUC_{inf}, AUCₑₓₜᵣₐₚ and T_{1/2} values are for 4-chloro-N-[2-[(4-chlorophenyl)methyl]-3-oxo-1,2,4-thiadiazol-5-yl]benzamide (Compound 1), which was detected following the administration of the compounds.

The results show that administration of Compounds 5, 6, 7, 9, 10, 13, 14, 16 and 17 provides mean plasma maximum concentrations (Cmax) of 4-chloro-N-[2-[(4-chlorophenyl)methyl]-3-oxo-1,2,4-thiadiazol-5-yl]benzamide in the range of 71 to 137 μg/mL.

TABLE 7
Mean Plasma Pharmacokinetic Parameters for 4-Chloro-N-[2-[(4-chlorophenyl)methyl]-3-oxo-1,2,4-
thiadiazol-5-yl]benzamide Following Administration of Compounds 5, 6, 7, 9, 10, 13, 14, 16 and 17

| Compound No. | 5 | 6 | 7 | 9 | 10 | 13 | 14 | 16 | 17 |
|---|---|---|---|---|---|---|---|---|---|
| Dose (mg/kg b.w.) | 50 | 50 | 50 | 50 | 50 | 50 | 50 | 50 | 50 |
| Cₘₐₓ (μg/mL) | 111.67 ± | 137.00 ± | 137.33 ± | 127.67 ± | 114.03 ± | 71.57 ± | 139.00 ± | 121.67 ± | 117.00 ± |
| | 10.02 | 6.25 | 13.43 | 5.13 | 13.69 | 18.40 | 14.11 | 21.08 | 12.12 |
| Tₘₐₓ (h) | 6.67 ± | 5.33 ± | 8.00 ± | 6.67 ± | 5.33 ± | 8.00 ± | 5.33 ± | 4.00 ± | 5.33 ± |
| | 2.31 | 2.31 | 0.00 | 2.31 | 2.31 | 0.00 | 2.31 | 0.00 | 2.31 |
| AUCₗₐₛₜ (h*μg/mL) | 1624.43 ± | 2216.42 ± | 2120.27 ± | 1941.77 ± | 1745.15 ± | 1101.82 ± | 2206.93 ± | 1749.97 ± | 1790.23 ± |
| | 52.13 | 110.17 | 253.90 | 150.32 | 163.94 | 294.49 | 102.94 | 144.73 | 208.72 |
| AUC_{inf} (h*μg/mL) | 2252.24 ± | 3197.99 ± | 2759.61 ± | 2393.94 ± | 2120.39 ± | 1713.69 ± | 2726.32 ± | 2030.60 ± | 2377.90 ± |
| | 171.33 | 811.71 | 405.12 | 443.06 | 81.20 | 720.24 | 126.46 | 112.66 | 333.35 |
| AUCₑₓₜᵣₐₚ (%) | 27.54 ± | 28.52 ± | 22.93 ± | 17.80 ± | 17.81 ± | 33.17 ± | 19.03 ± | 13.90 ± | 24.55 ± |
| | 7.04 | 12.87 | 3.32 | 9.21 | 4.65 | 9.59 | 2.23 | 2.33 | 1.82 |
| T_{1/2} (h) | 13.77 ± | 13.97 ± | 11.39 ± | 9.79 ± | 9.63 ± | 14.39 ± | 9.86 ± | 8.21 ± | 11.58 ± |
| | 3.63 | 6.02 | 1.00 | 3.32 | 1.23 | 2.65 | 0.66 | 0.78 | 1.10 |

TABLE 8
Plasma Concentration (μg/mL) of Compound 1 Following Administration of Compounds 5, 6, 7, 9, 10, 13, 14, 16 and 17

| | Compound | Compound | Compound | Compound | Compound | Compound | Compound | Compound | Compound |
|---|---|---|---|---|---|---|---|---|---|
| Time (h) | 5 | 6 | 7 | 9 | 10 | 13 | 14 | 16 | 17 |
| 0.00 | 0.00 | 0.00 | 0.00 | 0.00 | 0.00 | 0.00 | 0.00 | 0.00 | 0.00 |
| 1.00 | 35.93 | 76.53 | 22.73 | 33.47 | 76.70 | 3.74 | 57.90 | 34.07 | 19.53 |
| 4.00 | 81.27 | 123.43 | 112.27 | 114.60 | 106.37 | 43.73 | 138.00 | 121.67 | 106.20 |
| 8.00 | 101.80 | 127.00 | 137.33 | 123.67 | 100.53 | 71.57 | 131.67 | 106.67 | 110.00 |
| 24.00 | 31.27 | 45.17 | 38.57 | 29.63 | 26.77 | 28.20 | 36.43 | 23.67 | 34.93 |

| | |
|---|---|
| Lower limit of quantification of Compound 1 | 0.5 μg/mL |

### Example 26—Stability of Compound 17

The stability of Compound 17 was assessed in buffers with pH 1.20, 7.40 and 9.20. The pH 1.20 study was conducted using an HCl buffer. A phosphate buffer was used for the pH 7.40 medium, and an alkaline borate buffer was used for the pH 9.20 medium.

HCl buffer—Potassium chloride (1.491 g) was dissolved in 100 mL of Ultrapure water type-1 to get 0.2 M KCl solution. About 50 mL of 0.2 M KCl was taken in a beaker and the pH was adjusted to 1.20 using 0.2 M HCl solution. The final volume was made up to 200 mL using Ultrapure water type-1 and stored at room temperature (22±3° C.).

Phosphate buffer—Potassium phosphate monobasic (2.722 g) was dissolved in 100 mL of Ultrapure water type-1 to get 0.2 M solution. About 50 mL of 0.2 M solution was taken in a beaker and the pH was adjusted to 7.40 using 0.2 M NaOH solution. The final volume was made up to 200 mL using Ultrapure water type-1 and stored at room temperature (22±3° C.).

Alkaline borate buffer—Boric acid (1.237 g) and potassium chloride (1.491 g) were dissolved in 100 mL of Ultrapure water type-1 to get 0.2 M solution. About 50 mL of 0.2 M solution was taken in beaker and the pH was adjusted to 9.20 using 0.2 M NaOH solution. The final volume was made up to 200 mL using Ultrapure water type-1 and stored at room temperature (22±3° C.).

5 μM solutions of Compound 17 in each buffer were prepared with 5 mM DMSO stock solutions. The solutions were incubated at 22 t 3° C. for 120 minutes, with shaking at 400 rpm using a thermomixer.

After 0, 15, 30, 60 and 120 minute intervals, aliquots of sufficient volume of the compound solutions were taken, diluted and analysed to determine the stability of Compound 17 in each solution, as well as the amount of Compound 1 present. The amount of Compound 1 present was compared to the amount expected assuming full conversion of Compound 17 to Compound 1.

### Results

The results for the stability experiments are tabulated in Table 9 below.

TABLE 9
Compound 17 Stability Data in Various Buffers

| | Percentage of | | | Percentage of | | |
|---|---|---|---|---|---|---|
| | Compound 17 remaining | | | Compound 1 formation | | |
| Time | compared to 0 minutes | | | compared to standard | | |
| (min) | pH 1.20 | pH 7.40 | pH 9.20 | pH 1.20 | pH 7.40 | pH 9.20 |
| 0.0 | 100.00 | 100.00 | 100.00 | 4.54 | 87.80 | 85.62 |
| 15.0 | 76.19 | 0.13 | 0.00 | 3.03 | 69.71 | 92.53 |
| 30.0 | 82.37 | 0.39 | 0.00 | 3.42 | 74.14 | 88.95 |
| 60.0 | 73.33 | 0.47 | 0.00 | 3.17 | 64.06 | 90.12 |
| 120.0 | 79.04 | 2.33 | 0.00 | 4.40 | 68.90 | 103.36 |

The results show that Compound 17 possesses greatest stability in low pH environments (pH=1.2), but degrades rapidly at pH 7.40 and above.

### Example 27—Additional Stability Results

The stability of Compounds 3, 6, 9, 20 and 23 was tested using the same methods as described above for Compound 17. The amounts of each compound remaining at the 15 min and 30 min time points are shown in Table 10.

TABLE 10
Stability Data in Various Buffers

| Compound | Buffer | % remaining | % remaining |
|---|---|---|---|
| No. | pH | after 15 min | after 30 min |
| 3 | 1.2 | 97 | 83 |
| | 7.4 | 46 | 16 |
| 6 | 1.2 | 83 | 86 |
| | 7.4 | 33 | 33 |
| 9 | 1.2 | 100 | 100 |
| | 7.4 | 46 | 13 |
| 17 | 1.2 | 76 | 82 |
| | 7.4 | 0 | 0 |
| 20 | 1.2 | 88 | 99 |
| | 7.4 | 74 | 64 |
| 23 | 1.2 | 78 | 77 |
| | 7.4 | 0 | 0 |

Compounds 17 and 23 were found to degrade rapidly at pH 7.40, whilst remaining relatively stable at pH 1.2. These data suggest that, when the side chain of the amino acid(s) are lipophilic (e.g. alkyl, see Compound 20) then the stability is rather high at pH 7.4. The stability was lower when the side chain is H or a —NH₂ containing side chain (Glycine or Lysine based). When aspartic acid was used (Compound 17), the stability was very low at higher pH. Such compounds are likely to remain intact in the stomach but could be hydrolysed in the intestine.

1. A compound of formula I, 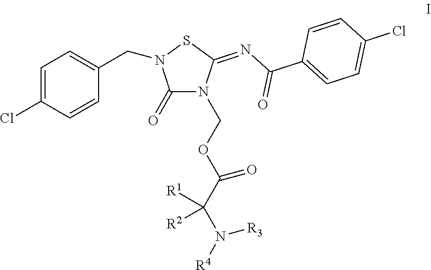
wherein:
R¹ and R² are independently selected from the group consisting of hydrogen, the sidechain of a proteinogenic amino acid, and C₁₋₆ alkyl optionally substituted by one or more substituents selected from the group consisting of —OH, —NH₂ and —C(O)NH₂;
R³ and R⁴ are independently selected from the group consisting of hydrogen, C₁₋₄ alkyl optionally substituted by a phenyl group and A¹; or
R¹ (or R²) and R³ (or R⁴) together with the carbon and nitrogen atoms to which they are bound form a 4- to 6-membered heterocycloalkyl group;
A¹ represents 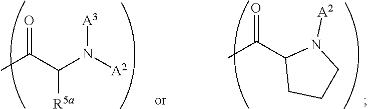
A² represents hydrogen, C₁₋₄ alkyl optionally substituted by a phenyl group, 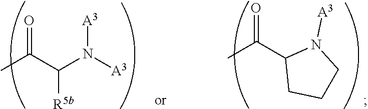
each A³ independently represents hydrogen or C₁₋₄ alkyl optionally substituted by a phenyl group; and
R^{5a} and R^{5b} are independently selected from the group consisting of hydrogen, the sidechain of a proteinogenic amino acid and C₁₋₆ alkyl optionally substituted by one or more substituents selected from the group consisting of —OH, —NH₂ and —C(O)NH₂,
or a pharmaceutically acceptable salt or solvate thereof.

2. The compound according to claim 1, or a pharmaceutically acceptable salt or solvate thereof, wherein R² is hydrogen, and R¹ is hydrogen or the sidechain of a proteinogenic amino acid selected from the group consisting of Glu, Arg, His, Lys, Ser, Thr, Asn, Gln, Cys, Sec, Ala, Ile, Leu, Met, Phe, Trp, Tyr, Asp and Val; or
R² is hydrogen, and R¹ and R³ together with the carbon and nitrogen atoms to which they are bound form a pyrrolidine ring.

3. The compound according to claim 1, or a pharmaceutically acceptable salt or solvate thereof, wherein R² is hydrogen, and R¹ is hydrogen or the sidechain of a proteinogenic amino acid selected from the group consisting of Arg, His, Lys and Trp; or
R² is hydrogen, and R¹ and R³ together with the carbon and nitrogen atoms to which they are bound form a pyrrolidine ring.

4. The compound according to claim 1, or a pharmaceutically acceptable salt or solvate thereof, wherein R² is hydrogen, and R¹ is the sidechain of aspartic acid.

5. The compound according to claim 1, or a pharmaceutically acceptable salt or solvate thereof, wherein R³ and R⁴ are independently selected from the group consisting of hydrogen, C₁₋₃ alkyl optionally substituted by one or more phenyl groups, 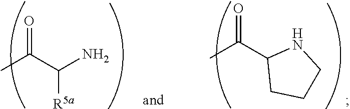
and
R^{5a} is hydrogen or the sidechain of a proteinogenic amino acid.

6. The compound according to claim 1, or a pharmaceutically acceptable salt or solvate thereof, wherein R³ and R⁴ are independently selected from the group consisting of hydrogen, C₁₋₃ alkyl optionally substituted by one or more phenyl groups, 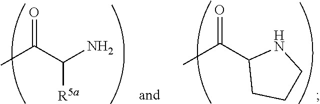
or
R¹ and R³ together with the carbon and nitrogen atoms to which they are bound form a pyrrolidine ring and R⁴ is selected from the group consisting of hydrogen, C₁₋₃ alkyl optionally substituted by one or more phenyl groups, 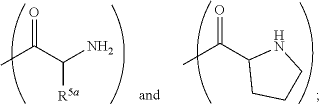
and
R^{5a} is the sidechain of a proteinogenic amino acid selected from the group consisting of Phe, Val, Lys, Asp and Arg.

7. The compound according to claim 1, or a pharmaceutically acceptable salt or solvate thereof, wherein R³ and R⁴ are independently selected from the group consisting of hydrogen and C₁₋₃ alkyl optionally substituted by one or more phenyl groups.

8. The compound according to claim 1, or a pharmaceutically acceptable salt or solvate thereof, wherein R³ and R⁴ are independently selected from the group consisting of hydrogen and methyl.

9. The compound according to claim 1, or a pharmaceutically acceptable salt or solvate thereof, wherein R² is hydrogen, R¹ is hydrogen or the sidechain of a proteinogenic amino acid selected from the group consisting of Glu, Arg, His, Lys, Ser, Thr, Asn, Gln, Cys, Sec, Ala, Ile, Leu, Met, Phe, Trp, Tyr, Asp and Val, R³ and R⁴ are independently selected from the group consisting of hydrogen, C₁₋₃ alkyl optionally substituted by one or more phenyl groups, 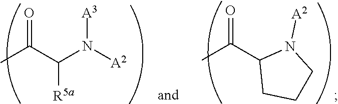
or
R² is hydrogen, R¹ and R³ together with the carbon and nitrogen atoms to which they are bound form a pyrrolidine ring, and R⁴ is selected from the group consisting of hydrogen, C₁₋₃ alkyl optionally substituted by one or more phenyl groups, 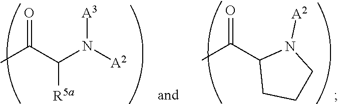
and
R^{5a} is hydrogen or the sidechain of a proteinogenic amino acid selected from the group consisting of Glu, Arg, His, Lys, Ser, Thr, Asn, Gln, Cys, Sec, Ala, Ile, Leu, Met, Phe, Trp, Tyr, Asp and Val.

10. A compound selected from the group consisting of:
dimethylamino-acetic acid 5-[(Z)-4-chloro-benzoylimino]-2-(4-chloro-benzyl)-3-oxo-[1,2,4]thiadiazolidin-4-ylmethyl ester;
citric acid salt of dimethylamino-acetic acid 5-[(Z)-4-chloro-benzoylimino]-2-(4-chloro-benzyl)-3-oxo-[1,2,4]thiadiazolidin-4-ylmethyl ester;
2-({[5-(4-chlorobenzamido)-2-[(4-chlorophenyl)methyl]-3-oxo-1,2,4-thiadiazolidin-4-yl]methoxy}carbonyl)pyrrolidin-1-ium trifluoroacetate;
6-{[5-(4-chlorobenzamido)-2-[(4-chlorophenyl)methyl]-3-oxo-1,2,4-thiadiazolidin-4-yl]methoxy}-6-oxohexane-1,5-bis(aminium) di-trifluoroacetate;
(6-azaniumyl-1-{[5-(4-chlorobenzamido)-2-[(4-chlorophenyl)methyl]-3-oxo-1,2,4-thiadiazolidin-4-yl]methoxy}-1-oxohexan-2-yl)dimethylazanium di-trifluoroacetate;
(2-{[5-(4-chlorobenzamido)-2-[(4-chlorophenyl)methyl]-3-oxo-1,2,4-thiadiazolidin-4-yl]methoxy}-2-oxoethyl)(methyl)azanium trifluoroacetate;
2-{[5-(4-chlorobenzamido)-2-[(4-chlorophenyl)methyl]-3-oxo-1,2,4-thiadiazolidin-4-yl]methoxy}-2-oxoethan-1-aminium trifluoroacetate;
2-({[5-(4-chlorobenzamido)-2-[(4-chlorophenyl)methyl]-3-oxo-1,2,4-thiadiazolidin-4-yl]methoxy}carbonyl)-1-methylpyrrolidin-1-ium trifluoroacetate;
1-{[5-(4-chlorobenzamido)-2-[(4-chlorophenyl)methyl]-3-oxo-1,2,4-thiadiazolidin-4-yl]methoxy}-1-oxopropan-2-aminium trifluoroacetate;
1-{[5-(4-chlorobenzamido)-2-[(4-chlorophenyl)methyl]-3-oxo-1,2,4-thiadiazolidin-4-yl]methoxy}-3-methyl-1-oxopentan-2-aminium trifluoroacetate;
[5-(4-chlorobenzamido)-2-[(4-chlorophenyl)methyl]-3-oxo-1,2,4-thiadiazolidin-4-yl]methyl 2-(dimethylamino)-3-phenylpropanoate;
1-{[5-(4-chlorobenzamido)-2-[(4-chlorophenyl)methyl]-3-oxo-1,2,4-thiadiazolidin-4-yl]methoxy}-1-oxo-3-phenylpropan-2-aminium trifluoroacetate;
1-{[5-(4-chlorobenzamido)-2-[(4-chlorophenyl)methyl]-3-oxo-1,2,4-thiadiazolidin-4-yl]methoxy}-4-methyl-1-oxopentan-2-aminium trifluoroacetate;
1-{[5-(4-chlorobenzamido)-2-[(4-chlorophenyl)methyl]-3-oxo-1,2,4-thiadiazolidin-4-yl]methoxy}-3-hydroxy-1-oxopropan-2-aminium trifluoroacetate;
3-carboxy-1-{[5-(4-chlorobenzamido)-2-[(4-chlorophenyl)methyl]-3-oxo-1,2,4-thiadiazolidin-4-yl]methoxy}-1-oxopropan-2-aminium trifluoroacetate;
[5-(4-chlorobenzamido)-2-[(4-chlorophenyl)methyl]-3-oxo-1,2,4-thiadiazolidin-4-yl]methyl 2-[(2S)-2-azaniumyl-3-phenylpropanamido]acetate chloride;
[5-(4-chlorobenzamido)-2-[(4-chlorophenyl)methyl]-3-oxo-1,2,4-thiadiazolidin-4-yl]methyl (2S)-2-[(2S)-2-azaniumyl-3-methylbutanamido]-3-methylbutanoate chloride;
[5-(4-chlorobenzamido)-2-[(4-chlorophenyl)methyl]-3-oxo-1,2,4-thiadiazolidin-4-yl]methyl (2R)-2-[(2R)-2-azaniumyl-3-methylbutanamido]-3-methylbutanoate chloride;
[5-(4-chlorobenzamido)-2-[(4-chlorophenyl)methyl]-3-oxo-1,2,4-thiadiazolidin-4-yl]methyl 2-[(2S)-2,6-diazaniumylhexanamido]acetate dichloride;
(1S)-2-carboxy-1-[(2-{[5-(4-chlorobenzamido)-2-[(4-chlorophenyl)methyl]-3-oxo-1,2,4-thiadiazolidin-4-yl]methoxy}-2-oxoethyl)carbamoyl]ethan-1-aminium chloride; and
[azaniumyl({[(4S)-4-azaniumyl-4-[(2-{[5-(4-chlorobenzamido)-2-[(4-chlorophenyl)methyl]-3-oxo-1,2,4-thiadiazolidin-4-yl]methoxy}-2-oxoethyl)carbamoyl]butyl]amino})methylidene]azanium trichloride, or a pharmaceutically acceptable salt or solvate thereof.

11. A pharmaceutical formulation comprising a compound as defined in a claim 1, or a pharmaceutically acceptable salt or solvate thereof, and a pharmaceutically acceptable excipient.

12-14. (canceled)

15. A method of treating a disorder or condition ameliorated by the activation of AMPK comprising administering a compound of formula I as defined in claim 1, or a pharmaceutically acceptable salt or solvate thereof, to a subject in need thereof.

16. The method according to claim 15, wherein the disorder or condition ameliorated by the activation of AMPK is selected from the group consisting of cardiovascular disease, diabetic kidney disease, type 2 diabetes, insulin resistance, non-alcoholic fatty liver disease, non-alcoholic steatohepatitis, pain, opioid addiction, obesity, cancer, inflammation, autoimmune diseases, osteoporosis, intestinal diseases and hyperinsulinemia associated with obesity or cardiovascular disease.

17. A process for preparing a compound as defined in claim 1, which process comprises:
(i) reaction of a compound of formula III, 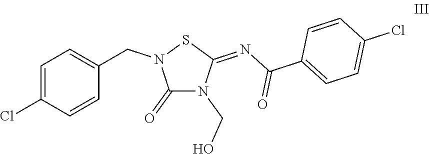 [SMILES: O=C(/N=c1/sn(Cc2ccc(Cl)cc2)c(=O)n1CO)c1ccc(Cl)cc1]
with a compound of formula IV, 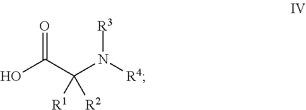
or
(ii) reaction of a compound of formula V, 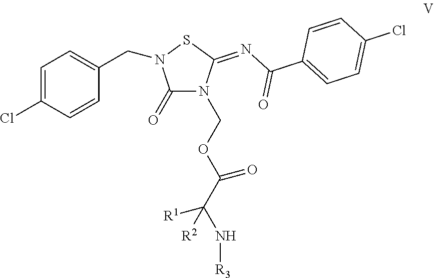
with a compound of formula IV, 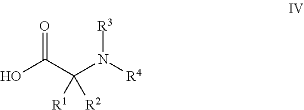
wherein R¹ and R² are as defined in claim 1; and
R³ and R⁴ are as defined in claim 1 or independently represent a protecting group.

18. The method accordingly to claim 16, wherein the cardiovascular disease is heart failure.

19. The method accordingly to claim 16, wherein the inflammation includes chronic inflammatory diseases.
